(19) United States
(12) Patent Application Publication
(10) Pub. No.: US 2013/0005684 A1
(43) Pub. Date: Jan. 3, 2013

(54) PHARMACEUTICAL COMPOSITION CONTAINING CYCLODEXTRIN COPOLYMER

(76) Inventors: Thomas Fichert, Warendorf (DE); Ingo Bichlmaier, Helsinki (FI)

(21) Appl. No.: 13/635,228

(22) PCT Filed: Mar. 18, 2011
(86) PCT No.: PCT/EP2011/001355

§ 371 Date: Sep. 14, 2012

(30) Foreign Application Priority Data
May 22, 2010 (DE) 10 2010 012 282.3

(51) Int. Cl. A61K 31/724; A61P 13/12; C08B 37/16

(52) U.S. Cl. 514/58; 536/103

(57) ABSTRACT

The present invention relates to cyclodextrin copolymers, processes for preparing same and their use to prepare pharmaceutical compositions.

The present invention relates to cyclodextrin copolymers, processes for producing same and use of same to produce pharmaceutical compositions.

Osmotically active compounds (osmotics) are widely used in pharmacy and medicine. For example, osmotics are used to adjust the tonicity of drugs, in particular parenteral medications. The osmotic pressure of a drug is adjusted to be hypotonic, hypertonic or isotonic, depending on how it is used. For example, the osmotic pressure of a parenteral pharmaceutical solution may be adapted to the osmotic pressure of human blood by adding an osmotic agent (isosmotic solutions).

Furthermore, osmotics are used in dialysis therapy, in particular in peritoneal dialysis, to withdraw excess water from the dialysis patient.

The peritoneal dialysis process is based on the introduction of a solution containing osmotically active compounds through a catheter into the abdominal cavity of the dialysis patient. This solution is left in the patient's abdominal cavity for a certain period of time (usually a few hours), manifesting its osmotic effect there, i.e., endogenous water is withdrawn from the patient into the abdominal cavity. After a certain dwell time, the peritoneal dialysis solution, which is now dilute, is drained out through a catheter.

This principle is employed in various processes for peritoneal dialysis therapy. For example, the processes for intermittent peritoneal dialysis (IPD), nocturnal intermittent peritoneal dialysis (NIPD), continuous cyclic peritoneal dialysis (CCPD) or continuous ambulant peritoneal dialysis (CAPD) may be used as needed. Devices which support the patient in performing the peritoneal dialysis process are used in IPD, NIPD and CAPD. CAPD is a manual process.

The addition of osmotically active compounds should in particular ensure that the osmotic pressure of the peritoneal dialysis solution is high enough during the entire dwell time in the abdominal cavity to withdraw water from the patient, i.e., water goes from the patient's circulation into his abdominal cavity (ultrafiltration).

However, because of the transfer of water into the abdominal cavity the peritoneal dialysis solution thereby introduced is necessarily diluted. This dilution results in a decline in the concentration of the osmotically active compound and thus also a decline in the osmotic pressure of this solution.

If the osmotic pressure of the peritoneal dialysis solution drops due to this dilution, this in turn results in a decline in the transfer of water into the abdominal cavity taking place per unit of time, or this process may even come to a complete standstill. In these cases, i.e., with a progressive dwell time of the peritoneal dialysis solution in the patient's abdominal cavity, effective water withdrawal is no longer taking place.

The direction of the transfer of water may even be reversed due to absorption of osmotically active compounds into the patient's bloodstream, i.e., water goes from the abdominal cavity into the patient's bloodstream (negative ultrafiltration). This is the case when the dilute peritoneal dialysis solution in the abdominal cavity has a lower osmotic pressure than the patient's endogenous water (e.g., blood).

By adding suitable osmotically active compounds to the peritoneal dialysis solution, the osmotic pressure can be maintained for a treatment, which is suitable for peritoneal dialysis, so there is no excessive decline in ultrafiltration within the dwell time of the solution in the abdominal cavity. Thus negative ultrafiltration is also largely prevented.

The solutions used in peritoneal dialysis treatment usually contain sugar monomers or polymers such as glucose or polyglucose (e.g., starch derivatives) as osmotically active compounds.

U.S. Pat. No. 4,889,634 relates to a peritoneal dialysis solution containing hydroxylpropyl-β-cyclodextrin.

JP8071146 relates to a peritoneal dialysis solution containing α- or γ-cyclodextrin, 2-hydroxyethyl ether, 2-hydroxypropyl ether, 6-O-α-glucosyl or 6-O-α-maltosyl derivates of α-, β- and γ-cyclodextrin.

The object of the present invention is to make available novel cyclodextrin copolymers, which may be used as osmotics, in particular in dialysis solutions.

This object is achieved by the subject matter of the patent claims.

The subject matter of this invention is copolymers containing at least two cyclodextrin monomers and at least one linker for use in the treatment of diseases of the urogenital system.

The inventive copolymers preferably contain at least two unsubstituted or mono- or polysubstituted cyclodextrin monomers, which may be the same or different, and one or more unsubstituted or mono- or polysubstituted linkers, which may be the same or different.

In a preferred embodiment, the inventive copolymers contain the same or different cyclodextrin monomers and the same or different linkers, where the proportional quantity of substance of the cyclodextrin monomer amounts to 5.0 to 80 mol % and the proportional quantity of substance of the linker amounts to 20 to 95 mol %, based on the total quantity of substance of the monomers contained in the copolymer.

The term “copolymer” in the sense of this invention stands for macromolecular compounds having a molecular weight of ≧2.0 kDa. The inventive copolymers may be random, alternating, block or crosslinked copolymers (for the definitions of these terms, reference is made to A. D. Jenkins, P. Kratochvil, R. F. T. Stepto, U. W. Suter. Glossary of Basic Terms in Polymer Science (IUPAC Recommendations 1996), Pure Appl. Chem., 1996, 68(12), 2287-2311).

In the sense of this invention, the term “copolymer” stands for a single inventive copolymer as well as a mixture containing two or more different inventive copolymers, e.g., 3, 4, 5, 6 or 7, or an indeterminable number of different inventive copolymers. For the purposes of this invention, the term “different copolymers” includes, for example, inventive copolymers of a different chemical structure, which contain different monomers or are isomers of one another and/or are different from one another in their molecular weight, degree of polymerization, total degree of substitution, melting point, ¹H-NMR, ¹³C-NMR, IR and/or MS spectra.

For the purposes of the present invention, the term “cyclodextrin compound” is understood to include unsubstituted or mono- or polysubstituted α-, β- and γ-cyclodextrin.

Cyclodextrins are cyclic oligosaccharides, where the glucosyl units of the ring structure have 1→4 glycosidic bonds. The cyclodextrins are differentiated according to the size of the oligosaccharide ring, i.e., according to the number of glucosyl units contained in the ring structure. For example, α-cyclodextrin contains six glucosyl units in the cyclodextrin ring structure, β-cyclodextrin contains seven glucosyl units, γ-cyclodextrin contains eight and δ-cyclodextrin contains nine.

Cyclodextrins (i.e., α-, β-, γ-, and δ-cyclodextrin) are usually synthesized by biological (i.e., enzymatic) processes. The products obtained by means of these biological synthesis processes are usually mixtures containing cyclodextrins of different ring sizes. The products obtained by purification of these mixtures contain predominately cyclodextrin of a uniform ring size but may still contain quantities of cyclodextrins of a different ring size. This is due in particular to the similar physicochemical properties of cyclodextrins of different ring sizes and the associated difficulty of isolating a cyclodextrin of a certain ring size. For example, the β-cyclodextrin product with a purity of ≧98%, guaranteed by the manufacturer, may still contain small amounts (traces) of other cyclodextrins (i.e., α-cyclodextrin, γ-cyclodextrin, δ-cyclodextrin). For this reason, the inventive copolymers containing cyclodextrin monomers of certain ring sizes may also contain traces (small amounts) of cyclodextrin monomers of other ring sizes. For example, the inventive β-cyclodextrin-epichlorohydrin copolymer may also contain traces of α-, β-, γ- and/or δ-cyclodextrin monomers.

The phrase “traces of cyclodextrin monomers of other ring sizes” in the sense of this invention means that these other cyclodextrin monomers may be present in amounts of preferably ≦10 wt %, more preferably ≦5.0 wt %, even more preferably ≦2.5 wt %, most preferably ≦1.0 wt % and in particular ≦0.50 wt % in the inventive cyclodextrin copolymer, based on the total weight of the cyclodextrin monomers contained in the inventive cyclodextrin copolymer. The cyclodextrin monomers of other ring sizes may also be present in the inventive copolymer in analytically immeasurable amounts, where the phrase “analytically immeasurable” in the sense of this invention stands for quantities below the limit of detection of an analytical detection process (e.g., NMR, LC/MS).

For the purposes of this invention, the term “monomer” stands for the structural elements which are contained in copolymers and are derived from cyclodextrin compounds and crosslinking agents. For example, the term “linker” (crosslinking agent monomer) includes the structural elements derived from crosslinking agents, while the term “cyclodextrin monomer” includes the structural elements derived from cyclodextrin compounds.

For example, if the unsubstituted cyclodextrin compound α-cyclodextrin is reacted with the crosslinking agent epichlorohydrin, then the following α-cyclodextrin-epichlorohydrin copolymer can be obtained:

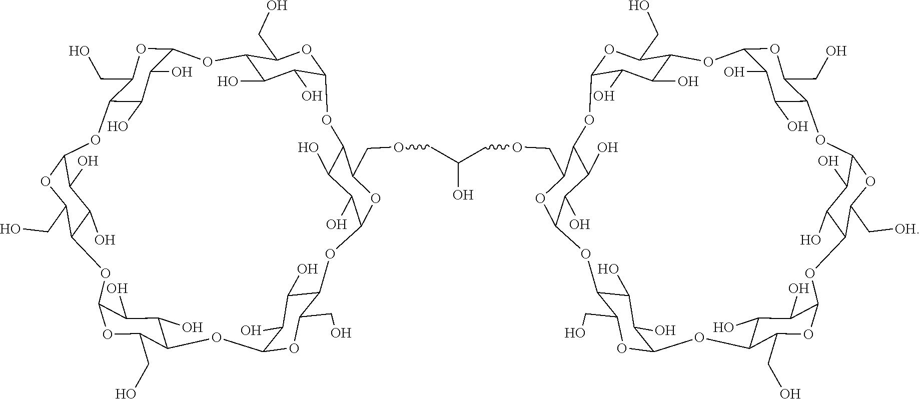 [SMILES: OCC1OC2OC3C(CO)OC(OC4C(CO)OC(OC5C(COCC(O)COCC6OC7OC8C(CO)OC(OC9C(CO)OC(OC%10C(CO)OC(OC%11C(CO)OC(OC%12C(CO)OC(OC6C(O)C7O)C(O)C%12O)C(O)C%11O)C(O)C%10O)C(O)C9O)C(O)C8O)OC(OC6C(CO)OC(OC7C(CO)OC(OC1C(O)C2O)C(O)C7O)C(O)C6O)C(O)C5O)C(O)C4O)C(O)C3O]

In the α-cyclodextrin-epichlorohydrin copolymer of this example, the structural element

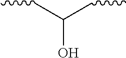

is the linker and/or the epichlorohydrin linker, and
the structural element

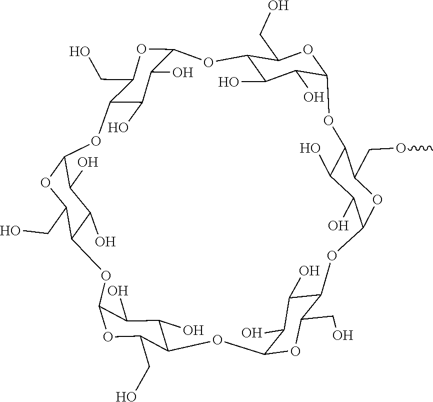

is the cyclodextrin monomer the structural and/or α-cyclodextrin element monomer,
where the symbol [symbol] stands for a covalent bond between the linker and the cyclodextrin monomer.

In the sense of this invention, the term “crosslinked” stands for the covalent bond of at least two cyclodextrin monomers by at least one linker. The crosslinking is usually achieved by forming covalent bonds between a bifunctional or polyfunctional cross-linking agent and nucleophilic radicals of at least two cyclodextrin compounds. Nucleophilic radicals in the sense of this invention include, for example, oxygen atoms of unsubstituted hydroxyl groups of the glucosyl units of cyclodextrin compounds or hydrogen atoms of unsubstituted hydroxyl groups of hydroxyalkyl substituents, which are attached to cyclodextrin compounds. The crosslinking may thus take place via oxygen atoms of unsubstituted hydroxyl groups of the cyclodextrin ring system as well as via oxygen atoms of unsubstituted hydroxyl groups of corresponding substituents bound to the cyclodextrin compound. However, nitrogen atoms of primary, secondary and tertiary amino groups as well as sulfur atoms of unsubstituted thiol groups are also examples of nucleophilic radicals in the sense of this invention. These nucleophilic radicals are also referred to as “nucleophilic hydroxyl groups (—OH)”, “nucleophilic thiol groups (—SH)” and “nucleophilic amino groups.”

For the purposes of this invention, cyclodextrin monomers are unsubstituted or mono- or polysubstituted with the same or different radicals.

With respect to the cyclodextrin monomers and cyclodextrin compounds, “simple substitution” in the sense of this invention is understood to refer to a single substitution of a hydrogen radical by one of the unsubstituted hydroxyl groups present in the cyclodextrin monomers and cyclodextrin compounds.

With respect to the cyclodextrin monomers and cyclodextrin compounds, the term “polysubstituted” is understood to refer to multiple substitutions (e.g., two, three, four or five times) of the hydrogen radicals of multiple hydroxyl groups (e.g., 2, 3, 4 or 5) which are present in the cyclodextrin monomers and cyclodextrin compounds, by multiple substituents, which may be the same or different. In addition, however, the substituents (radicals) of the cyclodextrin monomers and cyclodextrin compounds may also be monosubstituted or polysubstituted.

With respect to the linkers, the terms “mono- or polysubstituted” and “substituted one or more times” in the sense of this invention are understood to refer to the substitution of one or more hydrogen radicals by one or more substituents, in particular the substitution of the hydrogen radical of a hydroxyl group or the substitution of the hydrogen radicals of multiple hydroxyl groups by one or more substituents, which are the same or different.

With respect to the substituents, the phrase “monosubstituted or polysubstituted” in the sense of this invention is understood to refer to substitution of the hydrogen radical of a hydroxyl group or thiol group or the substation of the hydrogen radicals of multiply hydroxyl groups or thiol groups by one or more substituents, which may be the same or different. In addition, the primary, secondary or tertiary amino groups also contained in the substituents and radicals may also be substituted.

For the purposes of this invention, the terms “diseases of the urogenital system” and “renal insufficiency” stand for medical indications such as those defined by the World Health Organization in the “International Statistical Classification of Diseases and Related Health Problems” (ICD, 10^{th} Revision, German Modification, Version 2009; hereinafter referred to as “ICD-10”). The indication “diseases of the urogenital system” here comprises sections N00 to N99, and the indication “renal insufficiency” comprises sections N17 to N19 of ICD-10.

The inventive copolymers are suitable for use in the treatment of diseases of the urogenital system and may preferably be used in the treatment of renal insufficiency. The inventive copolymers are suitable in particular for use in dialysis treatment (hemodialysis or peritoneal dialysis) and may be used in peritoneal dialysis treatment.

In the sense of the present invention, the symbol “≧” stands for “greater than or equal to,” the symbol “≦” stands for “less than or equal to,” the symbol “−” stands for a negative charge, and the symbol “+” stands for a positive charge.

In a preferred embodiment, the proportional amount of substance of the linkers contained in the inventive copolymer is 20 to 95 mol %, more preferably 25 to 80 mol %, even more preferably 30 to 70 mol %, most preferably 30 to 60 mol % and in particular 30 to 50 mol %, based on the total amount of substance of the monomers present in the inventive copolymer.

The proportional amount of substance of the cyclodextrin monomers contained in the inventive copolymer preferably amounts to 5.0 to 80 mol %, more preferably 10 to 75 mol %, even more preferably 20 to 70 mol %, most preferably 30 to 70 mol % and in particular 40 to 70 mol %, based on the total amount of substance of the monomers contained in the inventive copolymer.

In another preferred embodiment, the total of the proportional amount of substance of the linkers and the proportional amount of substance of the cyclodextrin monomers is ≧25 mol %, ≧30 mol % or ≧40 mol %, more preferably ≧50 mol % or ≧60 mol %, more preferably ≧70 mol % or ≧80 mol %, most preferably ≧90 mol % or ≧95 mol %, and in particular 100 mol %, based on the total amount of substance of the monomers contained in the inventive copolymer.

Because of impurities in the educts (starting materials), a value of exactly 100 mol % cannot usually be achieved in practice. The value 100 mol % is therefore to be understood in the sense of this invention as meaning that except for cyclodextrin compounds, crosslinking agents and optionally functionalizing agents, no other compounds have been used to produce the inventive copolymers.

The water solubility of the inventive copolymers at 23° C. is preferably ≧5.0 g l⁻¹, ≧10 g l⁻¹, ≧25 g l⁻¹ or ≧50 g l⁻¹, more preferably ≧75 g l⁻¹, ≧100 g l⁻¹, ≧125 g l⁻¹ or ≧150 g l⁻¹, even more preferably ≧175 g l⁻¹, ≧200 g l⁻¹, ≧225 g l⁻¹ or ≧250 g l⁻¹, most preferably ≧300 g l⁻¹ or ≧400 g l⁻¹ and in particular ≧500 g l⁻¹.

The osmolality of a 50 mM aqueous solution of the inventive copolymers is preferably ≧50 mosm kg⁻¹, ≧75 mosm kg⁻¹ or ≧100 mosm kg⁻¹, more preferably 125 mosm kg⁻¹, ≧150 mosm kg⁻¹ or ≧175 mosm kg⁻¹, even more preferably ≧200 mosm kg⁻¹ or ≧225 mosm kg⁻¹, most preferably ≧250 mosm kg⁻¹ or ≧275 mosm kg⁻¹ and in particular ≧300 mosm kg⁻¹.

The degree of polymerization of the inventive copolymers is preferably ≧2.0 or ≧3.0, more preferably ≧4.0 or ≧5.0, even more preferably ≧6.0 or ≧7.0, most preferably ≧8.0 or ≧9.0 and in particular ≧10.

For the purposes of this invention, the term “degree of polymerization” stands for the following quotient:

$$Polymerisationsgrad = \frac{M ( qCD )}{M ( CDM )} ,$$

degree of polymerization
   where M(qCD) stands for the average molecular weight of the inventive copolymers, and M(CDM) stands for the average molecular weight of the cyclodextrin monomer.

The average molecular weight of the inventive copolymers is preferably ≧2.0 kDa, more preferably ≧2.5 kDa, even more preferably ≧3.0 kDa, most preferably kDa and in particular ≧5.0 kDa. The molecular weight is preferably measured with the help of a membrane osmometer (distilled water, 23° C.).

In another embodiment, the average molecular weight (M) of the inventive polymers is preferably 2.0≦M≦100 kDa, more preferably 2.0≦M≦50 kDa, even more preferably 2.0≦M≦25 kDa, most preferably 2.0≦M≦15 kDa and in particular 2.0≦M≦10 kDa.

In another preferred embodiment, the average particle diameter of the inventive copolymers is ≦1000 μm, ≦900 μm or ≦800 μm, more preferably ≦700 μm, ≦600 μm or ≦500 μm, even more preferably ≦400 μm, ≦300 μm or ≦200 μm, most preferably ≦100 μm, ≦75 μm or ≦50 μm and in particular ≦25 μm.

In a preferred embodiment, the cyclodextrin monomers may be the same or different and are derived from α-, β- or γ-cyclodextrin, each being unsubstituted or mono- or polysubstituted. In the sense of this invention, unsubstituted, monosubstituted or polysubstituted α-, β- and γ-cyclodextrins are referred to as “cyclodextrin compounds.”

In the sense of this invention, the term “cyclodextrin monomers derived from cyclodextrin compounds” stands for structural elements contained in the inventive copolymers and attributable to the structural modification of the cyclodextrin compounds by the crosslinking reaction with crosslinking agents (in this regard, cf. also the example of a reaction given above).

For the purposes of this invention, the cyclodextrin monomers derived from cyclodextrin compounds are preferably unsubstituted, monosubstituted or polysubstituted monomers of the inventive copolymer, having at least one covalent bond to a linker (crosslinking agent monomer), i.e., for example, at least one of the hydrogen radicals of an unsubstituted hydroxyl group or thiol group or cyclodextrin compound is replaced by a covalent bond to a crosslinking agent monomer. In addition, the covalent bonding may also take place via the nitrogen atom of an amino group of substituted cyclodextrin compounds.

In the sense of this invention, the term “unsubstituted hydroxyl groups of the cyclodextrin compounds” stands for OH radicals in positions 2, 4 and/or 6 of each glucosyl unit of the cyclodextrin ring system, but also for hydroxyl groups contained in the substituents (radicals) of substituted cyclodextrin compounds.

For the purposes of this invention, the term “unsubstituted thiol groups of the cyclodextrin compounds” stands for SH radicals contained in the substituents (radicals) of the cyclodextrin compounds.

In the sense of this invention, the term “amino groups of substituted cyclodextrin compounds” includes primary (—NH₂), secondary [e.g., —NH(CH₃)] and tertiary amino groups [e.g., —N(CH₃)₂], which are contained in the substituents (radicals) of substituted cyclodextrin compounds, where the symbol “—” stands for the bond to a carbon atom of the corresponding substituent (radical).

The substituents (radicals) of the cyclodextrin compounds are preferably derived from one or more functionalizing agents, which may be the same or different and may also optionally be further substituted.

The substituents are preferably derived from functionalizing agents selected from the group consisting of C₁₋₆-alkyl-Y, C₃₋₇-cycloalkyl-Y, C₁₋₆-alkyl-C(═O)—Y, C₃₋₇-cycloalkyl-C(═O)—Y, Y—C₁₋₆-alkyl-C(═O)—OH, Y—C₃₋₇-cycloalkyl-C(═O)—OH, Y—C₃₋₇-cycloalkyl-C(═O)—O—C₃₋₇-cycloalkyl, [C₁₋₆-alkyl-C(═O)]₂O, [C₃₋₇-cycloalkyl-C(═O)]₂O, dihydrofuran-2,5-dione, furan-2,5-dione, dihydro-2H-pyran-2,6(3H)dione, oxirane, C₁₋₆-alkyloxirane, C₃₋₇-cycloalkyloxirane, di-C₁₋₆-alkyloxirane, di-C₃₋₇-cycloalkyloxirane, 2-chloroethanol, di-C₁₋₆-alkyl sulfate, di-C₃₋₇-cycloalkyl sulfate, di-C₁₋₆-alkylphosphorochloridate, di-C₃₋₇-cycloalkylphosphorochloridate, NH₃, (C₁₋₆-alkyl) NH₂, (C₃₋₇-cycloalkyl)NH₂, (C₁₋₆-alkyl)₂NH, (C₃₋₇-cycloalkyl)₂NH, (C₁₋₆-alkyl)₃N, (C₃₋₇-cycloalkyl)₃N, [(Y—C₁₋₆-alkyl)N(C₁₋₆-alkyl)₃]⁺ halide, 1,3-propane sultone and 1,4-butane sultone; where C₁₋₆-alkyl and C₃₋₇-cycloalkyl, independently of one another, may be unsubstituted or mono- or polysubstituted with the same or different radicals Z, where the additional substituents Z result in little or no significant crosslinking of the cyclodextrin monomers or compounds: “halide” preferably stands for chloride, bromide or iodide, especially preferably chloride or bromide and in particular chloride; and the radicals Y stand for leaving groups.

The leaving groups Y, independently of one another, are preferably selected from the group consisting of —Cl, —Br, —I, -mesyl, -tosyl and -epoxy; —Cl, —Br, —I, -mesyl and -tosyl are especially advantageous, in particular —Cl.

The Z substituents, independently of one another, are preferably selected form the group consisting of —OH, ═O, —C(═O)OH, —C(═O)OCH₃, —C(═O)OCH₂CH₃, —NH₂, —NH(CH₃), —N(CH₃)₂, —N(CH₃)₃⁺A⁻, —NH(CH₂CH₃), —N(CH₂CH₃)₂ and —N(CH₂CH₃)₃⁺A⁻; where A⁻ stands for an organic or inorganic anion, which is preferably selected from the group consisting of Cl⁻, Br⁻, I⁻, dihydrogen phosphate, hydrogen phosphate, phosphate, bicarbonate, carbonate, acetate, lactate and citrate or for other physiologically tolerable anions. Especially advantageous are —OH, ═O, —C(═O)OH, —C(═O)OCH₃ and —C(═O)OCH₂CH₃; in particular —OH, ═O and —C(═O)OH.

In another preferred embodiment, the substituents are derived from at least one, preferably only one functionalizing agent, selected from the group consisting of CH₃—Y¹, CH₃—CH₂—Y¹, CH₃—CH₂—CH₂—Y¹, (CH₃)₂CH—Y¹, CH₃—CH₂—CH₂—CH₂—Y¹, (CH₃)₂CH—CH₂—Y¹, CH₃—CH₂—CH(CH₃)—Y¹, (CH₃)₃C—Y¹, CH₂═CH—Y¹, CH₂═CH—CH₂—Y¹, cyclopropyl-Y¹, Y¹—CH₂—C(═O)—OH, Y¹—CH₂—CH₂—C(═O)—OH, Y¹—CH(CH₃)—C(═O)—OH, Y¹—CH₂—C(═O)—O—CH₃, Y¹—CH₂—CH₂—C(═O)—O—CH₃, Y¹—CH(CH₃)—C(═O)—O—CH₃, Y¹—CH₂—C(═O)—O—CH₂CH₃, Y¹—CH₂—CH₂—C(═O)—O—CH₂—CH₃, Y¹—CH(CH₃)—C(═O)—O—CH₂—CH₃, CH₃—C(═O)Y², CH₃—CH₂—C(═O)Y², (CH₃)₂CH—C(═O)Y², CH₃—CH₂—CH₂—C(═O)Y², (CH₃)₃C—C(═O)Y², CH₃—CH₂—CH(CH₃)—C(═O)Y², (CH₃)₂CH—CH₂—C(═O)Y², CH₂═CH—C(═O)Y², CH₂═CH—CH₂—C(═O)Y², cyclopropyl-C(═O)Y², dihydrofuran-2,5-dione, furan-2,5-dione, dihydro-2H-pyran-2,6(3H)-dione, [CH₃—C(═O)]₂O, [CH₃—CH₂—C(═O)]₂O, [CH₃—CH₂—CH₂—C(═O)]₂O, [(CH₃)₂CH—C(═O)]₂O, [CH₃—CH₂—CH₂—CH₂—C(═O)]₂O, [CH₃—CH₂—CH(CH₃)—C(═O)]₂O, [(CH₃)₂CH—CH₂—C(═O)]₂O, [(CH₃)₃C—C(═O)]₂O, [CH₂═CH—C(═O)]₂O, [CH₂═CH—CH₂—C(═O)]₂O, [cyclopropyl-C(═O)]₂O, CH₂═CH—C(═O)—OH, CH₃—CH═CH—C(═O)—OH, (CH₃)₂C═CH—C(═O)—OH, CH₃—CH₂—CH═CH—C(═O)—OH, CH₃—CH═C(CH₃)—C(═O)—OH, CH₂═CH—C(═O)—O—CH₃, CH₃—CH═CH—C(═O)—O—CH₃, (CH₃)₂C═CH—C(═O)—O—CH₃, CH₃—CH₂—CH═CH—C(═O)—O—CH₃, CH₃—CH═C(CH₃)—C(═O)—O—CH₃, CH₂═CH—C(═O)—O—CH₂—CH₃, CH₃—CH═CH—C(═O)—O—CH₂—CH₃, (CH₃)₂C═CH—C(═O)—O—CH₂—CH₃, CH₃—CH₂—CH═CH—C(═O)—O—CH₂—CH₃, CH₃—CH═C(CH₃)—C(═O)O—CH₂—CH₃, oxirane, 2-methyloxirane, 2-ethyloxirane, oxiran-2-yl-methanol, oxirane-2-carboxylic acid, oxirane-2-carboxylic acid methyl ester, oxirane-2-carboxylic acid ethyl ester, 2-(oxiran-2-yl)acetic acid, 2-(oxiran-2-yl)acetic acid methyl ester, 2-(oxiran-2-yl)acetic acid ethyl ester, Cl—CH₂—CH₂—OH, (CH₃-0)₂S(═O)₂, (CH₃—CH₂—O)₂S(═O)₂, (CH₃—O)₂P(═O)Cl, (CH₃—CH₂—O)₂P(═O)Cl, [(CH₃)₂CH—O]₂P(═O)Cl, NH₃, CH₃—NH₂, (CH₃)₂NH, (CH₃)₃N, CH₃—CH₂—NH₂, (CH₃—CH₂)₂NH, (CH₃—CH₂)₃N, [Cl—CH₂—CH₂—N(CH₃)₃]⁺Cl⁻, [Cl—CH₂—CH₂—N(CH₂—CH₃)₃]⁺Cl⁻, 1,3-propane sultone and 1,4-butane sultone; where the radicals Y¹, independently of one another, preferably stand for —Cl, —Br, —I, tosyl, mesyl or epoxy, especially preferably —Cl, —Br or epoxy and in particular —Cl; and the radicals Y², independently of one another, preferably stand for —Cl, —Br or —I, especially preferably for —Cl or —Br and in particular for —Cl.

In another preferred embodiment, the substituents are derived from at least one, preferably only one functionalizing agent, selected from the group consisting of CH₃—Y¹, CH₃—CH₂—Y¹, cyclopropyl-Y¹, Y¹—CH₂—C(═O)—OH, Y¹—CH₂—C(═O)—O—CH₃, Y¹—CH₂—C(═O)—O—CH₂—CH₃, CH₃—C(═O)Y², dihydrofuran-2,5-dione, furan-2,5-dione, dihydro-2H-pyran-2,6(3H)-dione, [CH₃—C(═O)]₂O, [cyclopropyl-C(═O)]₂O, oxirane, 2-methyloxirane, 2-ethyloxirane, oxiran-2-yl-methanol, oxirane-2-carboxylic acid, oxirane-2-carboxylic acid methyl ester, oxirane-2-carboxylic acid ethyl ester, 2-(oxiran-2-yl)acetic acid, 2-(oxiran-2-yl)acetic acid methyl ester, 2-(oxiran-2-yl)acetic acid ethyl ester, Cl—CH₂—CH₂—OH, (CH₃—O)₂S(═O)₂, (CH₃—CH₂—O)₂S(═O)₂, CH₃—NH₂, (CH₃)₂NH, (CH₃)₃N, CH₃—CH₂—NH₂, (CH₃—CH₂)₂NH, (CH₃—CH₂)₃N, [Cl—CH₂—CH₂—N(CH₃)₃]⁺Cl⁻, [Cl—CH₂—CH₂—N(CH₂—CH₃)₃]⁺Cl⁻, 1,3-propane sultone and 1,4-butane sultone; where the radicals Y¹, independently of one another, preferably stand for —Cl, —Br, —I, -tosyl, mesyl or epoxy, especially preferably for —Cl, —Br or epoxy and in particular for —Cl; and the radicals Y² independently of one another, preferably stand for —Cl, —Br or —I, especially preferably for —Cl or —Br and in particular for —Cl.

In another preferred embodiment, the substituents are derived from at least one, preferably only one functionalizing agent selected form the group consisting of 1,3-propane sultone, 1,4-butane sultone, chloroacetic acid, chloroacetic acid methyl ester and chloroacetic acid ethyl ester.

The substituents derived from functionalizing agents may be further substituted with hybrid substituents.

The cyclodextrin monomers are preferably structural elements of the general ring structures I, nor III

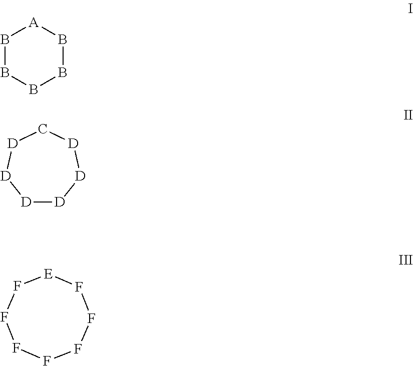

where
the glucosyl units A, C and E, independently of one another, are selected from the group consisting of

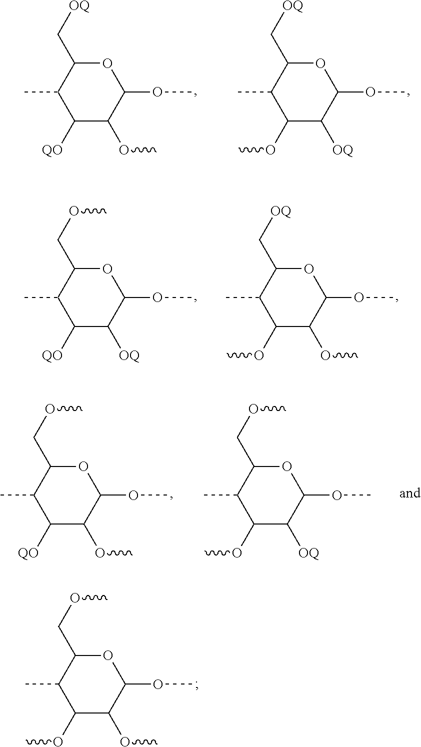

and
each individual one of the glucosyl units B, D and F, independently of one another, stands for either

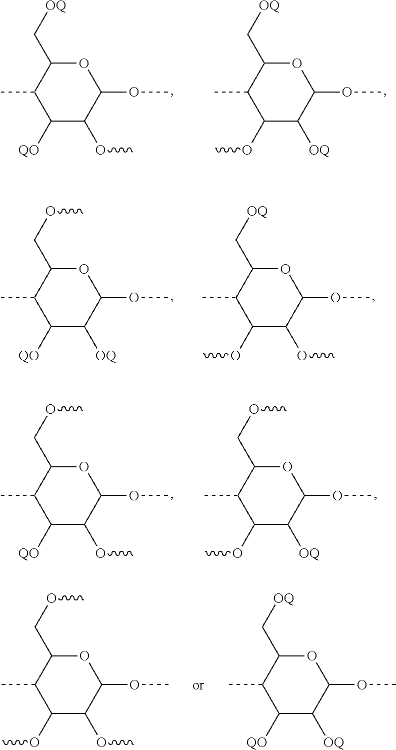

and
- - - - stands for the 1-4-glycosidic bonds in the cyclodextrin monomers;
[symbol] stands for the bond to a linker; where
   each individual radical Q stands either
   (i) for hydrogen or
   (ii) for a radical selected from the group consisting of glycosyl, —C(═O)R¹ and R²; where R¹ and R², independently of one another, stand for C₁₋₆-alkyl, C₃₋₇-cycloalkyl, phenyl or 5- to 7-membered heteroaryl.

In the sense of this invention, the α-cyclodextrin monomers are structural elements of general ring structure I; the β-cyclodextrin monomers are structural elements of general ring structure II; and the γ-cyclodextrin monomers structural elements of general ring structure III.

In another preferred embodiment, the cyclodextrin monomers, independently of one another, are selected from the group consisting of

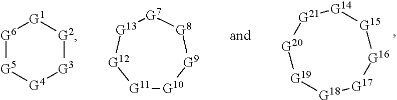

where
G¹, G⁷ and G¹⁴, independently of one another, are selected from the group consisting of

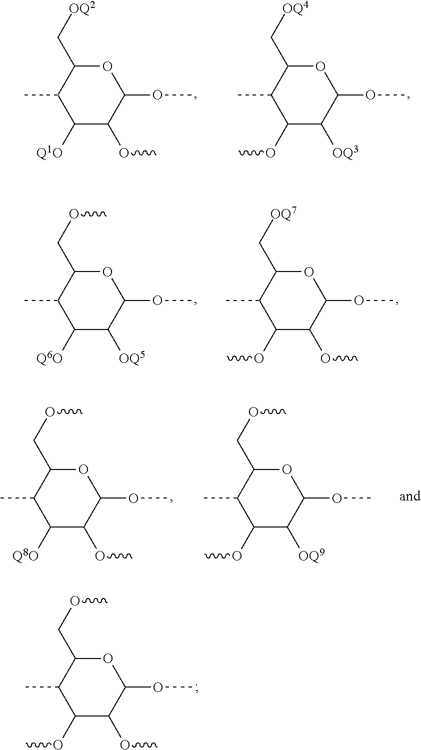

and
G², G³, G⁴, G⁵ G⁶, G⁸, G⁹, G¹⁰, G¹¹, G¹², G¹³, G¹⁶, G¹⁶, G¹⁷, G¹⁸, G¹⁹, G²⁰ and G²¹, independently of one another, are selected from the group consisting of

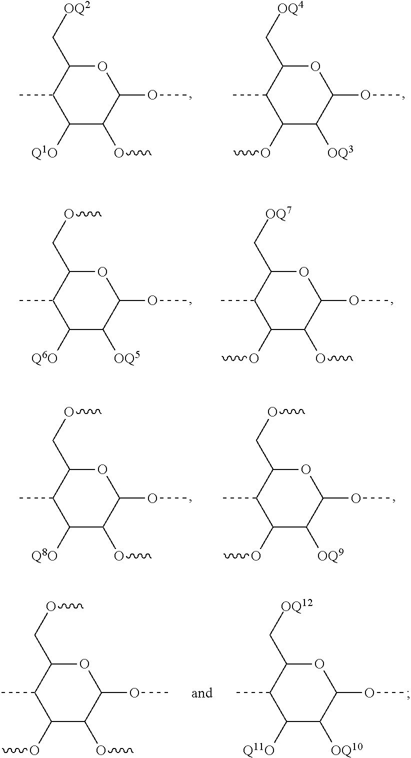

where
- - - - stands for the 1→4-glycocidic bonds in the cyclodextrin monomers;
[symbol] stands for the bond to a linker, and
Q¹, Q², Q³, Q⁴, Q⁵, Q⁶, Q⁷, Q⁸, Q⁹ and Q¹⁰, independently of one another, stand for hydrogen, -glycosyl, —C(═O)R³ or —R⁴; where
   R³ and R⁴, independently of one another, stand for C₁₋₆-alkyl, C₃₋₇-cycloalkyl, phenyl or 5- to 7-membered heteroaryl.

For the purposes of this invention, the term “glycosyl” includes saccharides, i.e., sugars, sugar acids and sugar alcohols, which may be monomers (monosaccharide), dimers (disaccharides), trimers (trisaccharides), oligomers (oligosaccharides) or polymers (polysaccharides), where the bond to the cyclodextrin monomers may involve any possible ring member of the glycosyl. Typical monosaccharides which may be present in the inventive copolymers include, for example, mannitol, isomalt, lactitol, sorbitol, glucitol, xylitol, threitol, erythritol, arabitol, glyceraldehyde, erythrose, threose, ribose, arabinose, xylose, lyxose, allose, altrose, glucose, mannose, gulose, idose, galactose, talose, dihydroxyacetone, erythrulose, ribulose, xylulose, psicose, fructose, sorbose, tagatose, glucosamine, fucosamine, N-acetylmannosamine, apiose, gluconic acid, galacturonic acid, erythraric acid, ascorbic acid, glucuronic acid, abequose, apiose, cladinose, deoxyribose, deoxyglucose, digitalose, digitoxose, fucose, fucosamine, galactosamine, glucosamine, glucosaminitol, glycerol, glycerone, mannosamine, manno sugar acid, neuraminic acid, rhamnose, mucic acid, sedoheptulose, streptose, trehalosamine, trehalose, tartaric acid and glucaric acid.

In a preferred embodiment, the glycosyl is a monosaccharide selected from the group consisting of mannitol, isomalt, lactitol, sorbitol, glucitol, xylitol, threitol, erythritol, arabitol, glyceraldehyde, erythrose, threose, ribose, arabinose, xylose, lyxose, allose, altrose, glucose, mannose, gulose, idose, galactose, talose, dihydroxyacetone, erythrulose, ribulose, xylulose, psicose, fructose, sorbose, tagatose, glucosamine, fucosamine, N-acetylmannosamine, hamamelose, apiose, gluconic acid, galacturonic acid, erythraric acid, ascorbic acid, glucuronic acid, abequose, apiose, cladinose, deoxyribose, deoxyglucose, digitalose, digitoxose, fucose, fucosamine, galactosamine, glucosamine, glucosaminitol, glycerol, glycerone, mannosamine, mannosaccharic acid, neuraminic acid, rhamnose, mucic acid, sedoheptulose, streptose, trehalosamine, trehalose, tartaric acid and glucaric acid. Glucose is especially advantageous.

The glycosyl may also be a disaccharide, trisaccharide, oligosaccharide or polysaccharide, preferably containing the same or different monomers of the aforementioned monosaccharides.

In the sense of this invention, the term “disaccharide” includes glycosyls, preferably containing two monomers, which may be the same as or different from the monosaccharides listed above; the term “trisaccharide” includes glycosyls, preferably containing three monomers, which may be the same as or different from the monosaccharides listed above; the term “oligosaccharide” includes glycosyls, preferably containing 4, 5, 6, 7, 8 or 9 monomers, which may be the same as or different from the monosaccharides listed above; and the term “polysaccharide” includes glycosyls, preferably containing ≧10 (i.e., 10, 11, 12, 13, 14, ≧15) monomers, which may be the same as or different from the monosaccharides listed above. The monosaccharide monomers contained in disaccharides, trisaccharides, oligosaccharides and polysaccharides are preferably linked to one another by glycosidic bonds, preferably by 1→2-glycosidic, 1→3-glycosidic, 1→4-glycosidic and/or 1→6-glycosidic bonds. Especially advantageous glycosyls are the monosaccharides glucose and galactose, the disaccharide maltose and the trisaccharide maltotriose. The bonding of the glycosyl to the cyclodextrin monomers and compounds may take place via any possible ring member of the glycosyl.

In a preferred embodiment, glycosyl is selected from the group consisting of mannitol, isomalt, lactitol, sorbitol, glucitol, xylitol, threitol, erythritol, arabitol, glyceraldehyde, erythrose, threose, ribose, arabinose, xylose, lyxose, allose, altrose, glucose, mannose, gulose, idose, galactose, talose, dihydroxyacetone, erythrulose, ribulose, xylulose, psicose, fructose, sorbose, tagatose, glucosamine, fucosamine, N-acetylmannosamine, hamamelose, apiose, gluconic acid, galacturonic acid, erythraric acid, ascorbic acid, glucuronic acid, abequose, apiose, cladinose, deoxyribose, deoxyglucose, digitalose, digitoxose, fucose, fucosamine, galactosamine, glucosamine, glucosaminitol, glycerol, glycerone, mannosamine, mannosaccharic acid, neuraminic acid, rhamnose, mucic acid, sedoheptulose, streptose, trehalosamine, trehalose, tartaric acid, glucaric acid, maltose and maltotriose. It is especially preferred for glycosyl to be selected from the group consisting of glucose, galactose, maltose and maltotriose.

The term “C₁₋₆-alkyl” in the sense of this invention includes acyclic, saturated or unsaturated hydrocarbon radicals which may be branched or linear and may contain 1, 2, 3, 4, 5 or 6 carbon atoms and may be unsubstituted or mono- or polysubstituted, where the substituents may be the same or different and may be in any possible position on the C₁₋₆-alkyl. Preferably C₁₋₆-alkyl includes the radicals C₁₋₆-alkanyl, C₂₋₆-alkenyl and C₂₋₆-alkynyl. The C₂₋₆-alkenyls have at least one C—C double bond and the C₂₋₆-alkynyls have at least one C—C triple bond, but the C₁₋₆-alkanyls are completely saturated. Preferably C₁₋₆-alkyl is selected from the group comprising methyl, ethyl, isopropyl, N-propyl, isobutyl, N-butyl, sec-butyl, tert-butyl, N-pentyl, isopentyl, neopentyl, isohexyl, N-hexyl, neohexyl, 3-methylpentyl, 2,3-dimethylbutyl, ethenyl, ethynyl, prop-1-enyl, isobutenyl, N-butenyl, cis-2-butenyl, trans-2-butenyl, 1,2-buta-dienyl, 1,3-butadienyl, pent-1-enyl, cis-pent-2-enyl, trans-pent-2-enyl, 2-methylbut-1-enyl, 2-methylbut-2-enyl, 3-methylbut-1-enyl, hex-1-enyl, hex-2-enyl and hex-3-enyl. The bond of C₁₋₆-alkyl radicals to cyclodextrin monomers and cyclodextrin compounds may involve any possible atom of the C₁₋₆-alkyls.

For the purposes of this invention, the term “C₃₋₇-cycloalkyl” includes cyclic hydrocarbons, which contain 3, 4, 5, 6 or 7 carbon atoms, may be saturated or mono- or polyunsaturated (but not aromatic) and may be unsubstituted or mono- or polysubstituted, where the substituents may be the same or different and may be in any possible position on the C₃₋₇-cycloalkyl. C₃₋₇-Cycloalkyl also includes saturated or unsaturated (but not aromatic) cycloalkyls, in which one or two carbon atoms are replaced by one or two heteroatoms, which may be the same or different and are selected from the group consisting of N, O and S. The C₃₋₇-cycloalkyl may be condensed with one or more saturated, unsaturated or aromatic ring systems. Preferably C₃₋₇-cycloalkyl is selected from the group comprising cyclopropyl, cyclobutyl, cyclopentyl, cyclohexyl, cycloheptyl, cyclopropenyl, cyclobutenyl, cyclopentenyl, cyclopentadienyl, cyclohexenyl, cyclohexadienyl, cycloheptenyl, cyclohepta-dienyl, cycloheptatrienyl, pyrrolinyl, pyrrolidinyl, tetrahydrofuranyl, pyrazolinyl, pyrazolidinyl, imidazolinyl, imidazolidinyl, dioxolanyl, piperidinyl, tetrahydropyranyl, piperazinyl, dioxanyl, morpholinyl, quinuclidinyl, pyrazolinyl, pyrazolinonyl, pyranyl, indolinyl, quinolizinyl, chromanyl, isochromanyl, chromenyl, isochromenyl and benzodioxolanyl. The bonds to the cyclodextrin monomers and cyclodextrin compounds may involve any possible ring member of the C₃₋₇-cycloalkyls. The C₃₋₇-cycloalkyl radical may also be bound to the cyclodextrin monomer via a C₁₋₄-alkylene bridge, preferably —CH₂—, —CH₂—CH₂—, —CH₂—CH₂—CH₂— or —CH₂—CH₂—CH₂—CH₂—.

In the sense of this invention, the term “C₁₋₄-alkylene bridge” includes structural elements bonding the C₃₋₇-cycloalkyl, phenyl and 5- to 7-membered heteroaryl radicals to the cyclodextrin monomers and cyclodextrin compounds. The C₁₋₄-alkylene bridge may be branched or linear, saturated (C₁₋₄-alkanylene bridge), or mono- or polyunsaturated (C₂₋₄-akenylene bridge, C₂₋₄-alkynylene bridge). The C₂₋₄-alkylene bridge contains at least one C—C double bond, and the C₂₋₄-alkynylene bridge contains at least one C—C triple bond. Preferably C₁₋₄-alkylene bridges are selected from the group consisting of —CH₂—, —CH₂—CH₂—, —CH₂—CH₂—CH₂— or —CH₂—CH₂—CH₂—CH₂—, —CH═CH—, —C═C—, —C═CH—CH₂—, —CH₂—CH═CH—CH₂— and —CH═CH—CH═CH—.

The term “phenyl” in the sense of this invention stands for an aromatic benzene radical, which may be condensed with additional saturated, unsaturated or aromatic ring systems (e.g., condensation of phenyl to naphthyl). The phenyl may be unsubstituted or mono- or polysubstituted, such that the substituents may be the same or different and may be in any possible position on the phenyl. The phenyl may be bound directly to the cyclodextrin monomers and cyclodextrin compounds via any possible ring member of the phenyl or may be bound via a C₁₋₄-alkylene bridge, preferably —CH₂—, —CH₂—CH₂—, —CH₂—CH₂—CH₂—, or —CH₂—CH₂—CH₂—CH₂—.

For the purposes of this invention, the term “5- to 7-membered heteroaryl” includes 5-, 6- or 7-membered cyclic aromatic radicals containing at least 1, optionally 2, 3, 4 or 5 heteroatoms, where the heteroatoms are the same or different, and the heteroaryl may be unsubstituted or mono- or polysubstituted. In the case of the substitution on the 5- to 7-membered heteroaryl, the substituents may be the same or different and may be in any possible position on the heteroaryl. Preferred heteroatoms include N, O and S. The heteroaryl may also be part of a bicyclic or polycyclic, carbocyclic or heterocyclic system. It is preferable for the 5- to 7-membered heteroaryl to be selected from the group consisting of pyrrolyl, pyrazolyl, imidazolyl, thienyl, furyl, oxazolyl, isoxazolyl, thiazolyl, isothiazolyl, furazanyl, oxadiazolyl, pyridyl, pyridazinyl, pyrazinyl, pyrimidinyl, indolyl, isoindolyl, indolizinyl, indazolyl, benzotriazolyl, quinolinyl, isoquinolinyl, naphthyridinyl, quinazolinyl, phthalazinyl, quinoxalinyl, purinyl, pteridinyl, benzofuryl, isobenzofuryl, benzothienyl, benzothiazolyl and benzothiadiazolyl. The bonding of the 5- to 7-membered heteroaryl radicals to the cyclodextrin monomers and cyclodextrin compounds may take place via any possible ring member of the heteroaryl. The 5- to 7-membered heteroaryl radical may also be bound to the cyclodextrin monomers and cyclodextrin compounds via a C₁₋₄-alkylene bridge, preferably —CH₂—, —CH₂—CH₂—, —CH₂—CH₂—CH₂—, or —CH₂—CH₂—CH₂—CH₂—.

With respect to C₁₋₆-alkyl, the term “monosubstituted or polysubstituted” in the sense of this invention refers to the substitution of one or more hydrogen radicals by one or more substituents, where alkyl radicals with multiple substituents are understood to be those substituted multiple times either on the same or different atoms, e.g., substituted two or more times, for example, twice on the same carbon atom, as in the case of —CHF₂, or in different positions, as in the case of —CH₂CH(OH)CH₂(OH). Multiple substitutions may involve the same or different substituents. One substituent may optionally also be substituted itself.

With respect to C₃₋₇-cycloalkyl, phenyl and 5- to 7-membered heteroaryl, the terms “mono- or polysubstituted” or “substituted one or more times” in the sense of this invention are understood to refer to substitution one or more times (e.g., two, three, four or five times) of one or more hydrogen atoms of the ring system and one atom or on different atoms. Multiple substitutions here involve the same or different substituents.

In the case of multiple substitution of a cyclodextrin-monomer, this may contain two or more (three, four or five) radicals, which are the same or different and are selected from the group consisting of glycosyl, —C(═O)R¹ and R² or the group consisting of glycosyl, —C(═O)R³ and R⁴. In the case of multiple substitutions of a cyclodextrin compound, it may contain two or more (e.g., three, four or five) radicals, which are the same or different and are selected from the group consisting of glycosyl, —C(═O)—R⁶ and R⁷.

With respect to the radicals R¹, R², R³ and R⁴, C₁₋₆-alkyl is preferably selected from the group consisting of methyl, ethyl, isopropyl, N-propyl, isobutyl, N-butyl, sec-butyl, tert-butyl, N-pentyl, isopentyl, neopentyl, isohexyl, N-hexyl, neohexyl, 3-methylpentyl, 2,3-dimethylbutyl, ethenyl, ethynyl, prop-1-enyl, isobutenyl, N-butenyl, cis-2-butenyl, trans-2-butenyl, 1,2-butadienyl, 1,3-butadienyl, pent-1-enyl, cis-pent-2-enyl, trans-pent-2-enyl, 2-methylbut-1-enyl, 2-methylbut-2-enyl, 3-methylbut-1-enyl, hex-1-enyl, hex-2-enyl and hex-3-enyl. It is especially preferred for C₆-alkyl to be selected from the group consisting of methyl, ethyl, isopropyl, N-propyl, isobutyl, N-butyl, sec-butyl, tert-butyl, N-pentyl, isopentyl, neopentyl, isohexyl, N-hexyl, neohexyl, 3-methylpentyl, 2,3-dimethylbutyl, ethenyl, ethynyl and prop-1-enyl. The aforementioned C₁₋₆-alkyl radicals may be unsubstituted or mono- or polysubstituted (e.g., two, three, four or five times) with R⁵ radicals.

With respect to the radicals R¹, R², R³ and R⁴, C₃₋₇-cycloalkyl is preferably selected from the group consisting of cyclopropyl, cyclobutyl, cyclopentyl, cyclohexyl, cycloheptyl, cyclopropenyl, cyclobutenyl, cyclopentenyl, cyclopentadienyl, cyclohexenyl, cyclohexadienyl, cycloheptenyl, cycloheptadienyl, cycloheptatrienyl, pyrrolinyl, pyrrolidinyl, tetrahydrofuranyl, pyrazolinyl, pyrazolidinyl, imidazolinyl, imidazolidinyl, dioxolanyl, piperidinyl, tetrahydropyranyl, piperazinyl, dioxanyl, morpholinyl, quinuclidinyl, pyrazolinyl, pyrazolinonyl, pyranyl, indolinyl, quinolizinyl, chromanyl, isochromanyl, chromenyl, isochromenyl and benzodioxolanyl. It is especially preferable for C₃₋₇-cycloalkyl to be selected from the group consisting of cyclopropyl, cyclobutyl, cyclopentyl, cyclohexyl, cycloheptyl, cyclopropenyl, cyclobutenyl, cyclopentenyl, pyrrolinyl, pyrrolidinyl, tetrahydrofuranyl, pyrazolinyl, pyrazolidinyl, imidazolinyl, imidazolidinyl, dioxolanyl, piperidinyl, tetrahydropyranyl, piperazinyl, dioxanyl, morpholinyl, quinuclidinyl, pyrazolinyl, pyrazolinonyl and pyranyl. It is also especially preferable for C₃₋₇-cycloalkyl to be selected from the group consisting of cyclopropyl, cyclobutyl, cyclopentyl, cyclohexyl, tetrahydrofuranyl, piperidinyl, piperazinyl, morpholinyl and quinuclidinyl. It is preferable in particular for C₃₋₇-cycloalkyl to be selected from the group consisting of cyclopropyl, cyclobutyl, cyclopentyl, cyclohexyl, piperidinyl, piperazinyl and morpholinyl. The aforementioned C₃₋₇-cycloalkyl radicals may be unsubstituted or mono- or polysubstituted (e.g., two, three, four or five times) with R⁵ radicals. The C₃₋₇-cycloalkyl radical may also be bound to the cyclodextrin monomers by a C₁₋₄-alkylene bridge, preferably —CH₂—, —CH₂—CH₂—, —CH₂—CH₂—CH₂—, or —CH₂—CH₂—CH₂—CH₂—.

With respect to the radicals R¹, R², R³ and R⁴, the phenyl radical may be unsubstituted or mono- or polysubstituted (e.g., two, three, four or five times) with R⁵ radicals. The phenyl radical may also be bound to the cyclodextrin monomers via a C₁₋₄-alkylene bridge, preferably —CH₂—, —CH₂—CH₂—, —CH₂—CH₂—CH₂—, or —CH₂—CH₂—CH₂—CH₂—.

With respect to the radicals R¹, R², R³ and R⁴ the 5- to 7-membered heteroaryl is preferably selected from the group consisting of pyrrolyl, pyrazolyl, imidazolyl, thienyl, furyl, oxazolyl, isoxazolyl, thiazolyl, isothiazolyl, furazanyl, oxadiazolyl, pyridyl, pyridazinyl, pyrazinyl, pyrimidinyl, indolyl, isoindolyl, indolizinyl, indazolyl, benzotriazolyl, quinolinyl, isoquinolinyl, naphthyridinyl, quinolinyl, phthalazinyl, quinoxalinyl, purinyl, pteridinyl, benzofuryl, isobenzofuryl, benzothienyl, benzothiazolyl and benzothiadiazolyl. In a preferred embodiment, 5- to 7-membered heteroaryl is selected from the group consisting of pyrrolyl, pyrazolyl, imidazolyl, thienyl, furyl, oxazolyl, isoxazolyl, thiazolyl, isothiazolyl, pyridyl, indolyl, quinolinyl, purinyl, pteridinyl and benzofuryl.

In an especially preferred embodiment, 5- to 7-membered heteroaryl is selected from the group consisting of pyrrolyl, pyrazolyl, imidazolyl, thienyl, furyl, oxazolyl, isoxazolyl, thiazolyl, isothiazolyl and pyridyl. The aforementioned 5-7-membered heteroaryl radicals may be unsubstituted or mono- or polysubstituted (e.g., two, three, four or five times) with R⁵ radicals. The 5- to 7-membered heteroaryl radical may also be bound to the cyclodextrin monomers via a C₁₋₄-alkylene bridge, preferably —CH₂—, —CH₂—CH₂—, —CH₂—CH₂—CH₂—, or —CH₂—CH₂—CH₂—CH₂—.

In a preferred embodiment, the substituents of the cyclodextrin monomers are selected from the group consisting of glucose, galactose, maltose, maltotriose, —C(═O)—C₁₋₆-alkyl, —C(═O)—C₃₋₇-cycloalkyl, —C(═O)-phenyl, —C(═O)-(5- to 7-membered heteroaryl), —C₁₋₆-alkyl, —C₃₋₇-cycloalkyl, phenyl and 5- to 7-membered heteroaryl, where the radicals —C₁₋₆-alkyl, —C₃₋₇-cycloalkyl, phenyl and 5- to 7-membered heteroaryl may be unsubstituted or mono- or polysubstituted (e.g., two, three, four or five times) with the same or different R⁵ radicals; and the C₃₋₇-cycloalkyl, phenyl and 5- to 7-membered heteroaryl radicals may be bound to the cyclodextrin monomers via a C₁₋₄-alkylene bridge, preferably —CH₂—, —CH₂—CH₂—, —CH₂—CH₂—CH₂—, or —CH₂—CH₂—CH₂—CH₂—.

The cyclodextrin monomers are preferably substituted with one or more radicals, which may be the same or different but are preferably the same and are selected from the group consisting of glucose, galactose, maltose, maltotriose, —C(═O)-methyl, —C(═O)-ethyl, —C(═O)-isopropyl, —C(═O)—N-propyl, —C(═O)-isobutyl, —C(═O)—N-butyl, —C(═O)-sec-butyl, —C(═O)-tert-butyl, —C(═O)—N-pentyl, —C(═O)-isopentyl, —C(═O)neopentyl, —C(═O)-isohexyl, —C(═O)—N-hexyl, —C(═O)-neohexyl, —C(═O)-(3-methylpentyl), —C(═O)-(2,3-dimethylbutyl), —C(═O)-ethenyl, —C(═O)-ethynyl, —C(═O)-prop-1-enyl, —C(═O)-cyclopropyl, —C(═O)-cyclobutyl, —C(═O)-cyclopentyl, —C(═O)-cyclohexyl, —C(═O)-piperidinyl, —C(═O)-piperazinyl, —C(═O)-morpholinyl, —C(═O)-phenyl, —C(═O)imidazolyl, —C(═O)-thienyl, —C(═O)-furyl, —C(═O)-oxazolyl, —C(═O)-isoxazolyl, —C(═O)thiazolyl, —C(═O)-isothiazolyl, —C(═O)-pyridyl, methyl, ethyl, isopropyl, N-propyl, isobutyl, N-butyl, sec-butyl, tert-butyl, N-pentyl, isopentyl, neopentyl, isohexyl, N-hexyl, neohexyl, 3-methylpentyl, 2,3-dimethylbutyl, ethenyl, ethynyl, prop-1-enyl, cyclopropyl, cyclobutyl, cyclopentyl, cyclohexyl, piperidinyl, piperazinyl, morpholinyl, phenyl, pyrrolyl, pyrazolyl, imidazolyl, thienyl, furyl, oxazolyl, isoxazolyl, thiazolyl, isothiazolyl and pyridyl; where the radicals may be unsubstituted or mono- or poly-substituted (e.g., two, three, four or five times) with R⁵ radicals; and the C₃₋₇-cycloalkyl, phenyl and 5- to 7-membered heteroaryl radicals may be bound to the cyclodextrin monomers via a C₁₋₄-alkylene bridge, preferably —CH₂—, —CH₂—CH₂—, —CH₂—CH₂—CH₂—, or —CH₂—CH₂—CH₂—CH₂—.

R⁵ is preferably selected from the group consisting of —C(═O)H, —C(═O)X, —C(═O)OH, —C(═O)OX, —C(═O)NH₂, —C(═O)NHX, —C(═O)NX₂, —C(═O)NHC(═O)X, —C(═O)NXC(═O)X, —OH, —OX, —OT, —OC(═O)H, —OC(═O)X, —OC(═O)OH, —OC(═O)OX, —OC(═O)—NH₂, —OC(═O)NHX, —OC(═O)NX₂, —O—N═O, —OS(═O)H, —OS(═O)X, —OS(═O)OH, —OS (═O)OX, —OS(═O)₂H, —OS(═O)₂X, —OS(═O)₂OH, —OS(═O)₂OX, —OS(═O)NH₂, —OS(═O)—NHX, —OS(═O)NX₂, —OS(═O)₂NH₂, —OS(═O)₂NHX, —OS(═O)₂NX₂, —OP(═O)(X)₂, —OP(═O)(OH)(X), —OP(═O)(OX)(X), —OP(═O)(OH)₂, —OP(═O)(OX)(OH), —OP(═O)(OX)₂, —O—P(═NH)(X)₂, —OP(═NX)(X)₂, —OP(═O)(NH₂)₂, —OP(═O)(NX)(NH₂), —OP(═S)(X)₂, —OP(═S)(OH)(X), —OP(═S)(OX)(X), —OP(═S)(OH)₂, —OP(═S)(OX)(OH), —OP(═S)(OX)₂, —SH, —SX, —ST, —S(═O)H, —S(═O)X, —S(═O)OH, —S(═O)OX, —S(═O)₂H, —S(═O)₂X, —S(═O)₂OH, —S(═O)₂OX, —S(═O)NH₂, —S(═O)NHX, —S(═O)NX₂, —S(═O)₂NH₂, —S(═O)₂NHX, —S(═O)₂NX₂, —NH₂, —NHX, —NHT, —NX₂, —NT₂, —N(X)₃⁺A⁻, —NHC(═O)H, —NHC(═O)X, —N[C(═O)X]₂, —NXC(═O)X, —NHC(═O)NH₂, —NHC(═O)NHX, NXC(═O)NHX, —NXC(═O)NX₂, —NXC(═O)NX₂, —NHC(═O)OH, —NHC(═O)OX, —NXC(═O)OX, —NHS(═O)H, —NHS(═O)X, —NXS(═O)H, —NXS(═O)X, —NHS(═O)OH, —NHS(═O)OX, —NXS(═O)OH, —NXS(═O)OX, —NHS(═O)₂H, —NHS(═O)₂X, —NXS(═O)₂H, —NXS(═O)₂X, —NHS(═O)₂OH, —NHS(═O)₂OX, —NXS(═O)₂OH, —NXS(═O)₂OX, —NHS(═O)NH₂, —NXS(═O)NHX, —NHS(═O)NX₂, —NXS(═O)NH₂, —NXS(═O)NX₂, —NHS(═O)₂NH₂, —NHS(═O)₂NHX, —NHS(═O)₂NX₂, —NXS(═O)₂NH₂, —NXS(═O)₂NHX, —NXS(═O)₂NX₂, ═N—OH, ═N—NH₂, —N═N—X, —PH₂, —PHX, —PX₂, —P(═O)X₂, —P(═O)(OH)(X), —P(═O)(OX)(X), —P(═O)(OH)₂, —P(═O)(OX) (OH), —P(═O) (OX)₂, —P(═NH)(X)₂, —P(═NX)(X)₂, —P(═O)(NH₂)₂, —P(═O)(NX₂)₂, —P(═S) (X)₂, —P(═S) (OH)(X), —P(═S)(OX)X, —P(═S)(OH)₂, —P(═S)(OX)(OH), —P(═S) (OX)₂, ═NH, ═NX, ═O, ═S, pyrrolidinyl, pyrazolinyl, pyrazolidinyl, imidazolinyl, imidazolidinyl, dioxolanyl, piperidinyl, piperazinyl, 4-x-piperazinyl, dioxanyl, morpholinyl, quinuclidinyl, pyrazolinonyl, indolinyl, isoindolinyl, quinolizinyl, benzodioxolanyl, pyrrolyl, pyrazolyl, imidazolyl, thienyl, oxazolyl, isoxazolyl, thiazolyl, isothiazolyl, furazanyl, oxadiazolyl, pyridyl, pyridazinyl, pyrazinyl, pyrimidinyl, indolyl, isoindolyl, indolizinyl, indazolyl, benzotriazolyl, quinolinyl, isoquinolinyl, naphthyridinyl, quinazolinyl, phthalazinyl, quinoxalinyl, purinyl, pteridinyl, benzothienyl, benzothiazolyl and benzothiadiazolyl; where the X radical preferably stands for methyl, ethyl, N-propyl, isopropyl, isobutyl, N-butyl, sec-butyl, tert-butyl, cyclopropyl, cyclobutyl, cyclopentyl, cyclohexyl, allyl, vinyl or ethynyl; the T radical stands for a hybrid radical; and A⁻ stands for an organic or inorganic anion preferably selected from the group consisting of Cl⁻, Br⁻, I⁻, dihydrogen phosphate, hydrogen phosphate, phosphate, bicarbonate, carbonate, acetate, lactate and citrate or for other physiologically tolerable anions.

In another preferred embodiment, R⁵ is selected from the group consisting of —C(═O)H, —C(═O)X, —C(═O)OH, —C(═O)OX, —C(═O)NH₂, —C(═O)NHX, —C(═O)NX₂, —C(═O)NHC(═O)X, —C(═O)NXC(═O)X, —OH, —OX, —OT, —OC(═O)H, —OC(═O)X, —OC(═O)OH, —OC(═O)OX, —OC(═O)NH₂, —OC(═O)NHX, —OC(═O)NX₂, —OS(═O)H, —OS(═O)X, —OS(═O)OH, —OS(═O)OX, —OS(═O)₂H, —OS(═O)₂X, —OS(═O)₂OH, —OS(═O)₂OX, —OS(═O)NH₂, —OS(═O)NHX, —OS(═O)NX₂, —OS(═O)₂NH₂, —OS(═O)₂NHX, —OS(═O)₂NX₂, —OP(═O)(OH)(X), —OP(═O)(OH)₂, —OP(═O)(OX)(OH), —OP(═O)(OX)₂, —SH, —ST, —S(═O)H, —S(═O)X, —S(═O)OH, —S(═O)OX, —S(═O)₂H, —S(═O)₂X, —S(═O)₂OH, —S(═O)₂OX, —S(═O)NH₂, —S(═O)NHX, —S(═O)NX₂, —S(═O)₂NH₂, —S(═O)₂NHX, —S(═O)₂NX₂, —NH₂, —NHX, —NHT, —NX₂, —NT₂, —N(X)₃⁺A⁻, —NHC(═O)H, —NHC(═O)X, —N[C(═O)X]₂, —NXC(═O)X, —NHC(═O)NH₂, —NHC(═O)NHX, —NXC(═O)NX₂, —NXC(═O)NHX, —NXC(═O)NX₂, —NHC(═O)OH, —NHC(═O)OX, —NXC(═O)OX, —NHS(═O)H, —NXS(═O)H, —NHS(═O)OH, —NXS(═O)OH, —NHS(═O)₂OH, —NXS(═O)₂OH, ═N—OH, ═N—NH₂, —PH₂, —P(═O)X₂, —P(═O)(OH)(X), —P(═O)(OH)₂, —P(═O)(OX)(OH), —P(═O)(OX)₂, ═NH, ═NX, ═O, ═S, pyrrolidinyl, pyrazolinyl, pyrazolidinyl, imidazolinyl, imidazolidinyl, piperidinyl, piperazinyl, 4-X-piperazinyl, morpholinyl, quinuclidinyl, pyrazolinonyl, imidazolyl; where the X radical preferably stands for methyl, ethyl, N-propyl, isopropyl, cyclopropyl, allyl, vinyl or ethynyl; the T radical stands for a hybrid radical; and A⁻ stands for an organic or inorganic anion, which is preferably selected from the group consisting of Cl⁻, Br⁻, I⁻, dihydrogen phosphate, hydrogen phosphate, phosphate, bicarbonate, carbonate, acetate, lactate and citrate or for other physiologically tolerable anions.

In another preferred embodiment, R⁵ stands for an ionic, ionizable hydroxyl or polyol radical. In this context, R⁵ is preferably selected from the group consisting of —C(═O)OH, —OH, —OT, —OC(═O)OH, —OC(═O)NH₂, —OS(═O)OH, —OS(═O)₂OH, —OP(═O)(OH)(X), —OP(═O)(OH)₂, —OP(═O)(OX)(OH), —OP(═O)(OX)₂, —SH, —ST, —S(═O)OH, —S(═O)₂OH, —NH₂, —NHX, —NHT, —NX₂, —NT₂, —N(X)₃⁺A⁻, —NHC(═O)OH, —NXC(═O)OH, —NHS(═O)OH, —NXS(═O)OH, —NHS(═O)₂OH, —NXS(═O)₂OH, ═N—OH, ═N—NH₂, —P(═O)(OH)(X), —P(═O)(OH)₂, —P(═O)(OX)(OH), —P(═O)(OX)₂, ═NH, ═NX, piperidinyl, morpholinyl and imidazolyl; where the X radical is selected from the group consisting of methyl, ethyl, N-propyl, isopropyl, cyclopropyl, allyl, vinyl and ethynyl; the T radical stands for a hybrid radical; where A⁻ stands for an organic or inorganic anion, which is preferably selected from the group consisting of Cl⁻, Br⁻, I⁻, dihydrogen phosphate, hydrogen phosphate, phosphate, bicarbonate, carbonate, acetate, lactate and citrate or for other physiologically tolerable anions.

In another preferred embodiment, R⁵ stands for an ionic, ionizable hydroxyl- or polyol radical. In this context, R⁵ is preferably selected from the group consisting of —C(═O)OH, —OH, —OT, —OS(═O)₂OH, —OP(═O)(OH)₂, —SH, —ST, —S(═O)₂OH, —NH₂, —NHX, —NHT, —NX₂, —NT₂, —N(X)₃⁺A⁻, —NHC(═O)OH, —NXC(═O)OH, —P(═O)(OH)(X), —P(═O)(OH)₂, —P(═O)(OX)(OH), where the X radical is selected from the group consisting of methyl, ethyl, N-propyl, isopropyl, cyclopropyl, allyl, vinyl and ethynyl; the T radical stands for a hybrid radical; where A⁻ stands for an organic or inorganic anion, which is preferably selected from the group consisting of Cl⁻, Br⁻, I⁻, dihydrogen phosphate, hydrogen phosphate, phosphate, bicarbonate, carbonate, acetate, lactate and citrate or for other physiologically tolerable anions.

In another preferred embodiment, the radicals R¹, R², R³ and R⁴, independently of one another, are selected from the group consisting of methyl, ethyl, isopropyl, N-propyl, isobutyl, N-butyl, sec-butyl, tert-butyl, ethenyl, ethynyl, prop-1-enyl, cyclopropyl, cyclobutyl, cyclopentyl, cyclohexyl, phenyl, benzyl, phenethyl, —CH₂—C(═O)—OH, —(CH₂)₂—C(═O)—OH, —(CH₂)₃—C(═O)—OH, —(CH₂)₄—C(═O)—OH, —(CH₂)₅—C(═O)—OH, —(CH₂)₆—C(═O)—OH, —CH₂—CH(OH)—C(═O)OH, —CH₂—CH(OH)—C(═O)O—CH₃, —CH₂—CH(OH)—C(═O)O—CH₂—CH₃, —CH(CH₂OH)—C(═O)OH, —CH(CH₂OH)—C(═O)O—CH₃, —CH(CH₂OH)—C(═O)O—CH₂—CH₃, —CH₂—CH(OH)—CH₂—C(═O)—OH, —CH₂—CH(OH)—CH₂—C(═O)—O—CH₃, —CH₂—CH(OH)—CH₂—C(═O)—OCH₂—CH₃, —CH(CH₂—OH)—CH₂—C(═O)—O—CH₃, —CH(CH₂—OH)—CH₂—C(═O)—O—CH₂—CH₃, —CH₂—OH, —CH(OH)—CH₃, —(CH₂)₂—OH, —CH(OH)—CH₂—CH₃, —CH₂—CH(OH)—CH₃, —(CH₂)₃—OH, —CH(CH₃)—CH₂—OH, —CH(OH)—(CH₂)₂—CH₃, —CH₂—CH(OH)—CH₂—CH₃, —(CH₂)₂—CH(OH)—CH₃, —(CH₂)₄—OH, —(CH₂)₅—OH, —(CH₂)₆—OH, —CH(OH)—CH₂(OH), —CH(OH)—CH(OH)—CH₃, —CH(OH)—CH₂—CH₂(OH), —CH₂—CH(OH)—CH₂(OH), —CH(OH)—CH(OH)—CH₂—CH₃, —CH(OH)—CH₂CH(OH)—CH₃, —CH(OH)—(CH₂)₂—CH₂(OH), —CH₂—CH(OH)—CH(OH)—CH₃, —CH₂—CH(OH)—CH₂—CH₂(OH), —(CH₂)₂—CH(OH)—CH₂(OH), —CH(OH)—CH(OH)—CH₂(OH), —CH(OH)—CH(OH)—CH(OH)—CH₃, —CH(OH)—CH(OH)—CH₂—CH₂(OH), —CH(OH)—CH₂—CH(OH)—CH₂(OH), —CH₂—CH(OH)—CH(OH)—CH₂(OH), —CH(OH)—CH(OH)—CH(OH)—CH₂(OH), —CH₂—O—S(═O)₂OH, —(CH₂)₂—O—S(═O)₂OH, —(CH₂)₃—O—S(═O)₂OH, —(CH₂)₄—O—S(═O)₂OH, —(CH₂)₅—O—S(═O)₂OH, —(CH₂)₆—O—S(═O)₂OH, —CH₂—O—P(═O)(OH)₂, —(CH₂)₂—O—P(═O)(OH)₂, —(CH₂)₃—O—P(═O)(OH)₂, —(CH₂)₄—O—P(═O)(OH)₂, —(CH₂)₅—O—P(═O)(OH)₂, —(CH₂)₆—O—P(═O)(OH)₂, —CH₂—SH, —CH(SH)—CH₃, —(CH₂)₂—SH, —CH(SH)—CH₂—CH₃, —CH₂—CH(SH)—CH₃, —(CH₂)₃—SH, —CH(SH)—(CH₂)₂—CH₃, —CH₂—CH(SH)—CH₂—CH₃, —(CH₂)₂—CH(SH)—CH₃, —(CH₂)₄—SH, —(CH₂)₅—SH, —(CH₂)₆—SH, —CH(SH)—CH₂(SH), —CH(SH)—CH(SH)—CH₃, —CH(SH)—CH₂—CH₂(SH), —CH₂—CH(SH)—CH₂(SH), —CH(SH)—CH(SH)—CH₂—CH₃, —CH(SH)—CH₂CH(SH)—CH₃, —CH(SH)—(CH₂)₂—CH₂(SH), —CH₂—CH(SH)—CH(SH)—CH₃, —CH₂—CH(SH)—CH₂—CH₂(SH), —(CH₂)₂—CH(SH)—CH₂(SH), —CH(SH)—CH(SH)—CH₂(SH), —CH(SH)—CH(SH)—C(SH)—CH₃, —CH(SH)—CH(SH)—CH₂—CH₂(SH), —CH(SH)—CH₂—CH(SH)—CH₂(SH), —CH₂—CH(SH)—CH(SH)—CH₂(SH), —CH(SH)—CH(SH)—CH(SH)—CHASED, —CH₂—S(═O)₂OH, —(CH₂)₂—S(═O)₂OH, —(CH₂)₃—S(═O)₂OH, —(CH₂)₄—S(═O)₂OH, —(CH₂)₅—S(═O)₂OH, —(CH₂)₆—S(═O)₂OH, —CH₂—NH₂, —(CH₂)₂—NH₂, —(CH₂)₃—NH₂, —(CH₂)₄—NH₂, —(CH₂)₅—NH₂, —(CH₂)₆—NH₂, —CH₂—NH(CH₃), —(CH₂)₂—NH(CH₃), —(CH₂)₃—NH(CH₃), —(CH₂)₄—NH(CH₃), —(CH₂)₅—NH(CH₃), —(CH₂)₆—NH(CH₃), —CH₂—NH(CH₂CH₃), —(CH₂)₂—NH(CH₂CH₃), —(CH₂)₃—NH(CH₂CH₃), —(CH₂)₄—NH(CH₂CH₃), —(CH₂)₅—NH(CH₂CH₃), —(CH₂)₆—NH(CH₂CH₃), —CH₂—N(CH₃)₂, —(CH₂)₂—N(CH₃)₂, —(CH₂)₃—N(CH₃)₂, —(CH₂)₄—N(CH₃)₂, —(CH₂)₅—N(CH₃)₂, —(CH₂)₆—N(CH₃)₂, —CH₂—N(CH₂CH₃)₂, —(CH₂)₂—N(CH₂CH₃)₂, —(CH₂)₃—N(CH₂CH₃)₂, —(CH₂)₄—N(CH₂—CH₃)₂, —(CH₂)₅—N(CH₂CH₃)₂, —(CH₂)₆—N(CH₂CH₃)₂, —CH₂—N(CH₃)₃⁺A⁻, —(CH₂)₂—N(CH₃)₃⁺A⁻, —(CH₂)₃—N(CH₃)₃⁺A⁻, —(CH₂)₄—N(CH₃)₃⁺A⁻, —(CH₂)₅—N(CH₃)₃⁺A⁻, —(CH₂)₆—N(CH₃)₃⁺A⁻, —CH₂—N(CH₂—CH₃)₃⁺A⁻, —(CH₂)₂—N(CH₂CH₃)₃⁺A⁻, —(CH₂)₃—N(CH₂CH₃)₃⁺A⁻, —(CH₂)₄—N(CH₂CH₃)₃⁺A⁻, —(CH₂)₅—N(CH₂CH₃)₃⁺A⁻, —(CH₂)₆—N(CH₂CH₃)₃⁺A⁻, —CH₂—P(═O)(OH)₂, —(CH₂)₂—P(═O)(OH)₂, —(CH₂)₃—P(═O)(OH)₂, —(CH₂)₄—P(═O)(OH)₂, —(CH₂)₅—P(═O)(OH)₂, —(CH₂)₆—P(═O)(OH)₂, —CH₂-pyrrolyl, —(CH₂)₂-pyrrolyl, —(CH₂)₃-pyrrolyl, —(CH₂)₄-pyrrolyl, —CH₂-pyrazolyl, —(CH₂)₂-pyrazolyl, —(CH₂)₃-pyrazolyl, —(CH₂)₄-pyrazolyl, —CH₂-imidazolyl, —(CH₂)₂-imidazolyl, —(CH₂)₃-imidazolyl, —(CH₂)₄-imidazolyl, —CH₂-oxazolyl, —(CH₂)₂-oxazolyl, —(CH₂)₃-oxazolyl, —(CH₂)₄-oxazolyl, —CH₂-isoxazolyl, —(CH₂)₂-isoxazolyl, —(CH₂)₃-isoxazolyl, —(CH₂)₄-isoxazolyl, —CH₂-isothiazolyl, —(CH₂)₂-isothiazolyl, —(CH₂)₃-isothiazolyl, —(CH₂)₄-isothiazolyl, —CH₂-pyridyl, —(CH₂)₂-pyridyl, —(CH₂)₃-pyridyl and —(CH₂)₄-pyridyl; where A⁻ stands for an organic or inorganic anion, which is preferably selected from the group consisting of Cl⁻, Br⁻, I⁻, dihydrogen phosphate, hydrogen phosphate, phosphate, bicarbonate, carbonate, acetate, lactate and citrate or for other physiologically tolerable anions; where the radicals may optionally be further substituted with hybrid radicals.

In another preferred embodiment, Q, Q¹, Q², Q³, Q⁴, Q⁵, Q⁶, Q⁷, Q⁸, Q⁹ and Q¹⁰, independently of one another, are selected from the group consisting of hydrogen, glucose, galactose, maltose, maltotriose, —C(═O)—C₃₋₇-cycloalkyl, —C(═O)-phenyl, —C(═O)-(5- to 7-membered heteroaryl), —C₁₋₆-alkyl, —C₃₋₇-cycloalkyl, -phenyl and -(5- to 7-membered heteroaryl); where the C₃₋₇-cycloalkyl, phenyl and 5- to 7-membered heteroaryl radicals may be bound to the cyclodextrin monomers via a C₁₋₄-alkylene bridge, preferably —CH₂—, —CH₂—CH₂—, —CH₂—CH₂—CH₂—, or —CH₂—CH₂—CH₂—CH₂—; and C₁₋₆-alkyl, C₃₋₇-cycloalkyl, phenyl and 5- to 7-membered heteroaryl may be unsubstituted or mono- or polysubstituted (e.g., two, three, four or five times) with the same or different R⁵ radicals; where R⁵ is selected from the group consisting of —C(═O)OH, —OH, —OT, —OC(═O)OH, —OC(═O)NH₂, —OS(═O)OH, —OS(═O)₂OH, —OP(═O)(OH)(X), —OP(═O)(OH)₂, —OP(═O)(OX)(OH), —OP(═O)(OX)₂, —SH, —ST, —S(═O)OH, —S(═O)₂OH, —NH₂, —NHX, —NHT, —NX₂, —NT₂, —N(X)₃⁺A⁻, —NHC(═O)OH, —NXC(═O)OH, —NHS(═O)OH, —NXS(═O)OH, —NHS(═O)₂OH, —NXS(═O)₂OH, ═N—OH, ═N—NH₂, —P(═O)(OH)(X), —P(═O)(OH)₂, —P(═O)(OX)(OH), —P(═O)(OX)₂, ═NH, ═NX, pyrrolidinyl, pyrazolinyl, pyrazolidinyl, imidazolinyl, imidazolidinyl, piperidinyl, piperazinyl, 4-X-piperazinyl, morpholinyl, quinuclidinyl, pyrazolinonyl and imidazolyl; where the X radical is selected from the group consisting of methyl, ethyl, N-propyl, isopropyl, cyclopropyl, allyl, vinyl and ethynyl; the T radical stands for a hybrid radical; where A⁻ stands for an organic or inorganic anion, which is preferably selected from the group consisting of Cl⁻, Br⁻, I⁻, dihydrogen phosphate, hydrogen phosphate, phosphate, bicarbonate, carbonate, acetate, lactate and citrate or for other physiologically tolerable anions.

In another preferred embodiment, Q, Q¹, Q², Q³, Q⁴, Q⁵, Q⁶, Q⁷, Q⁹ and Q¹⁰, independently of one another, are selected from the group consisting of hydrogen, glucose, galactose, maltose, maltotriose, —C(═O)-methyl, —C(═O)-ethyl, —C(═O)isopropyl, —C(═O)—N-propyl, —C(═O)-isobutyl, —C(═O)—N-butyl, —C(═O)-sec-butyl, —C(═O)-tert-butyl, —C(═O)—N-pentyl, —C(═O)-isopentyl, —C(═O)-neopentyl, —C(═O)isohexyl, —C(═O)—N-hexyl, —C(═O)-neohexyl, —C(═O)-(3-methylpentyl), —C(═O)-(2,3-dimethylbutyl), —C(═O)-ethenyl, —C(═O)-ethynyl, —C(═O)-prop-1-enyl, —C(═O)-cyclopropyl, —C(═O)-cyclobutyl, —C(═O)-cyclopentyl, —C(═O)-cyclohexyl, —C(═O)-piperidinyl, —C(═O)-piperazinyl, —C(═O)-morpholinyl, —C(═O)-phenyl, —C(═O)-imidazolyl, —C(═O)thienyl, —C(═O)-furyl, —C(═O)-oxazolyl, —C(═O)-isoxazolyl, —C(═O)-thiazolyl, —C(═O)isothiazolyl, —C(═O)-pyridyl, methyl, ethyl, isopropyl, N-propyl, isobutyl, N-butyl, sec-butyl, tert-butyl, N-pentyl, isopentyl, neopentyl, isohexyl, N-hexyl, neohexyl, 3-methylpentyl, 2,3-dimethylbutyl, ethenyl, ethynyl, prop-1-enyl, cyclopropyl, cyclobutyl, cyclopentyl, cyclohexyl, piperidinyl, piperazinyl, morpholinyl, phenyl, pyrrolyl, pyrazolyl, imidazolyl, thienyl, furyl, oxazolyl, isoxazolyl, thiazolyl, isothiazolyl and pyridyl; where the C₃₋₇-cycloalkyl, phenyl and 5- to 7-membered heteroaryl radicals may be bound to the corresponding cyclodextrin monomers via a C₁₋₄-alkylene bridge, preferably —CH₂—, —CH₂—CH₂—, —CH₂—CH₂—CH₂—, or —CH₂—CH₂—CH₂—CH₂—; and the C₁₋₆-alkyl, C₃₋₇-cycloalkyl, phenyl and 5- to 7-membered heteroaryl radicals may be unsubstituted or mono- or polysubstituted (e.g., two, three, four or five times) with R⁵ radicals; where R⁵ is preferably selected from the group consisting of —C(═O)OH, —OH, —OT, —OS(═O)₂OH, —OP(═O)(OH)₂, —SH, —ST, —S(═O)₂OH, —NH₂, —NHX, —NHT, —NX₂, —NT₂, —N(X)₃⁺A⁻—NHC(═O)OH, —NXC(═O)OH, —P(═O)(OH)(X), —P(═O)(OH)₂, —P(═O)(OX) (OH), piperidinyl, piperazinyl, morpholinyl and imidazolyl; where the X radical is selected from the group consisting of methyl, ethyl, N-propyl, isopropyl, cyclopropyl, allyl, vinyl and ethynyl; the T radical stands for a hybrid radical; where A⁻ stands for an organic or inorganic anion, preferably selected from the group consisting of Cl⁻, Br⁻, I⁻, dihydrogen phosphate, hydrogen phosphate, phosphate, bicarbonate, carbonate, acetate, lactate and citrate or other physiologically tolerable anions.

In another preferred embodiment, Q, Q¹, Q², Q³, Q⁴, Q⁵, Q⁶, Q⁷, Q⁵, Q⁹ and Q¹⁰, independently of one another, are selected from the group consisting of hydrogen, glucose, galactose, maltose, maltotriose, —C(═O)-methyl, —C(═O)-ethyl, —C(═O)isopropyl, —C(═O)—N-propyl, —C(═O)-isobutyl, —C(═O)—N-butyl, —C(═O)-sec-butyl, —C(═O)-tert-butyl, —C(═O)-ethenyl, —C(═O)-ethynyl, —C(═O)-prop-1-enyl, —C(═O)cyclopropyl, —C(═O)-cyclobutyl, —C(═O)-cyclopentyl, —C(═O)-cyclohexyl, —C(═O)phenyl, —C(═O)-benzyl, —C(═O)-phenethyl, —C(═O)—CH₂—C(═O)—OH, —C(═O)—(CH₂)₂—C(═O)—OH, —C(═O)—(CH₂)₃—C(═O)—OH, —C(═O)—(CH₂)₄—C(═O)—OH, —C(═O)—(CH₂)₅—C(═O)—OH, —C(═O)—(CH₂)₆—C(═O)—OH, —C(═O)—CH₂—OH, —C(═O)—CH(OH)—CH₃, —C(═O)—(CH₂)₂—OH, —C(═O)—CH(OH)—CH₂—CH₃, —C(═O)—CH₂—CH(OH)—CH₃, —C(═O)—(CH₂)₃—OH, —C(═O)—CH(OH)—(CH₂)₂—CH₃, —C(═O)—CH₂—CH(OH)—CH₂—CH₃, —C(═O)—(CH₂)₂—CH(OH)—CH₃, —C(═O)—(CH₂)₄—OH, —C(═O)—(CH₂)₅—OH, —C(═O)—(CH₂)₆—OH, —C(═O)—CH(OH)—CH₂(OH), —C(═O)—CH(OH)—CH(OH)—CH₃, —C(═O)—CH(OH)—CH₂—CH₂(OH), —C(═O)—CH₂—CH(OH)—CH₂(OH), —C(═O)—CH(OH)—CH(OH)—CH₂—CH₃, —C(═O)—CH(OH)—CH₂CH(OH)—CH₃, —C(═O)—CH(OH)—(CH₂)₂—CH₂(OH), —C(═O)—CH₂—CH(OH)—CH(OH)—CH₃, —C(═O)—CH₂—CH(OH)—CH₂—CH₂(OH), —C(═O)—(CH₂)₂—CH(OH)—CH₂(OH), —C(═O)—CH(OH)—CH(OH)—CH₂(OH), —C(═O)—CH(OH)—CH(OH)—CH(OH)—CH₃, —C(═O)—CH(OH)—CH(OH)—CH₂—CH₂(OH), —C(═O)—CH(OH)—CH₂—CH(OH)—CH₂(OH), —C(═O)—CH₂—CH(OH)—CH(OH)—CH₂(OH), —C(═O)—CH(OH)—CH(OH)—CH(OH)—CH₂(OH), —C(═O)—CH₂—NH₂, —C(═O)—(CH₂)₂—NH₂, —C(═O)—(CH₂)₃—NH₂, —C(═O)—(CH₂)₄—NH₂, —C(═O)—(CH₂)₅—NH₂, —C(═O)—(CH₂)₆—NH₂, —C(═O)—CH₂—NH(CH₃), —C(═O)—(CH₂)₂—NH(CH₃), —C(═O)—(CH₂)₃—NH(CH₃), —C(═O)—(CH₂)₄—NH(CH₃), —C(═O)—(CH₂)₅—NH(CH₃), —C(═O)—(CH₂)₆—NH(CH₃), —C(═O)—CH₂—NH(CH₂CH₃), —C(═O)—(CH₂)₂—NH(CH₂CH₃), —C(═O)—(CH₂)₃—NH(CH₂CH₃), —C(═O)—(CH₂)₄—NH(CH₂CH₃), —C(═O)—(CH₂)₅—NH(CH₂CH₃), —C(═O)—(CH₂)₆—NH(CH₂CH₃), —C(═O)—CH₂—N(CH₃)₂, —C(═O)—(CH₂)₂—N(CH₃)₂, —C(═O)—(CH₂)₃—N(CH₃)₂, —C(═O)—(CH₂)₄—N(CH₃)₂, —C(═O)—(CH₂)₅—N(CH₃)₂, —C(═O)—(CH₂)₆—N(CH₃)₂, —C(═O)—CH₂—N(CH₂—CH₃)₂, —C(═O)—(CH₂)₂—N(CH₂CH₃)₂, —C(═O)—(CH₂)₃—N(CH₂CH₃)₂, —C(═O)—(CH₂)₄—N(CH₂CH₃)₂, —C(═O)—(CH₂)₅—N(CH₂CH₃)₂, —C(═O)—(CH₂)₆—N(CH₂CH₃)₂, methyl, ethyl, isopropyl, N-propyl, isobutyl, N-butyl, sec-butyl, tert-butyl, ethenyl, ethynyl, prop-1-enyl, cyclopropyl, cyclobutyl, cyclopentyl, cyclohexyl, phenyl, benzyl, phenethyl, —CH₂—C(═O)—OH, —(CH₂)₂—C(═O)—OH, —(CH₂)₃—C(═O)—OH, —(CH₂)₄—C(═O)—OH, —(CH₂)₅—C(═O)—OH, —(CH₂)₆—C(═O)—OH, —CH₂—CH(OH)—C(═O)OH, —CH₂—CH(OH)—C(═O)O—CH₃, —CH₂—CH(OH)—C(═O)O—CH₂—CH₃, —CH(CH₂OH)—C(═O)OH, —CH(CH₂—OH)—C(═O)O—CH₃, —CH(CH₂OH)—C(═O)O—CH₂—CH₃, —CH₂—CH(OH)—CH₂—C(═O)—OH, —CH₂—CH(OH)—CH₂—C(═O)—O—CH₃, —CH₂—CH(OH)—CH₂—C(═O)—OCH₂—CH₃, —CH(CH₂—OH)—CH₂—C(═O)—O—CH₃, —CH(CH₂—OH)—CH₂—C(═O)—O—CH₂—CH₃, —CH₂—OH, —CH(OH)—CH₃, —(CH₂)₂—OH, —CH(OH)—CH₂—CH₃, —CH₂—CH(OH)—CH₃, —(CH₂)₃—OH, —CH(CH₃)—CH₂—OH, —CH(OH)—(CH₂)₂—CH₃, —CH₂—CH(OH)—CH₂—CH₃, —(CH₂)₂—CH(OH)—CH₃, —(CH₂)₄—OH, —(CH₂)₅—OH, —(CH₂)₆—OH, —CH(OH)—CH₂(OH), —CH(OH)—CH(OH)—CH₃, —CH(OH)—CH₂—CH₂(OH), —CH₂—CH(OH)—CH₂(OH), —CH(OH)—CH(OH)—CH₂—CH₃, —CH(OH)—CH₂—CH(OH)—CH₃, —CH(OH)—(CH₂)₂—CH₂(OH), —CH₂—CH(OH)—CH(OH)—CH₃, —CH₂—CH(OH)—CH₂—CH₂(OH), —(CH₂)₂—CH(OH)—CH₂(OH), —CH(OH)—CH(OH)—CH₂(OH), —CH(OH)—CH(OH)—CH(OH)—CH₃, —CH(OH)—CH(OH)—CH₂—CH₂(OH), —CH(OH)—CH₂—CH(OH)—CH₂(OH), —CH₂—CH(OH)—CH(OH)—CH₂(OH), —CH(OH)—CH(OH)—CH(OH)—CH₂(OH), —CH₂—O—S(═O)₂OH, —(CH₂)₂—O—S(═O)₂OH, —(CH₂)₃—O—S(═O)₂OH, —(CH₂)₄—O—S(═O)₂OH, —(CH₂)₅—O—S(═O)₂OH, —(CH₂)₆—O—S(═O)₂OH, —CH₂—O—P(═O)(OH)₂, —(CH₂)₂—O—P(═O)(OH)₂, —(CH₂)₃—O—P(═O)(OH)₂, —(CH₂)₄—O—P(═O)(OH)₂, —(CH₂)₅—O—P(═O)(OH)₂, —(CH₂)₆—O—P(═O)(OH)₂, —CH₂—SH, —CH(SH)—CH₃, —(CH₂)₂—SH, —CH(SH)—CH₂—CH₃, —CH₂—CH(SH)—CH₃, —(CH₂)₃—SH, —CH(SH)—(CH₂)₂—CH₃, —CH₂—CH(SH)—CH₂—CH₃, —(CH₂)₂—CH(SH)—CH₃, —(CH₂)₄—SH, —(CH₂)₅—SH, —(CH₂)₆—SH, —CH₂—S(═O)₂OH, —(CH₂)₂—S(═O)₂OH, —(CH₂)₃—S(═O)₂OH, —(CH₂)₄—S(═O)₂OH, —(CH₂)₅—S(═O)₂OH, —(CH₂)₆—S(═O)₂OH, —CH₂—NH₂, —(CH₂)₂—NH₂, —(CH₂)₃—NH₂, —(CH₂)₄—NH₂, —(CH₂)₅—NH₂, —(CH₂)₆—NH₂, —CH₂—NH(CH₃), —(CH₂)₂—NH(CH₃), —(CH₂)₃—NH(CH₃), —(CH₂)₄—NH(CH₃), —(CH₂)₅—NH(CH₃), —(CH₂)₆—NH(CH₃), —CH₂—NH(CH₂CH₃), —(CH₂)₂—NH(CH₂CH₃), —(CH₂)₃—NH(CH₂CH₃), —(CH₂)₄—NH(CH₂CH₃), —(CH₂)₅—NH(CH₂CH₃), —(CH₂)₆—NH(CH₂CH₃), —CH₂—N(CH₃)₂, —(CH₂)₂—N(CH₃)₂, —(CH₂)₃—N(CH₃)₂, —(CH₂)₄—N(CH₃)₂, —(CH₂)₅—N(CH₃)₂, —(CH₂)₆—N(CH₃)₂, —CH₂—N(CH₂CH₃)₂, —(CH₂)₂—N(CH₂CH₃)₂, —(CH₂)₃—N(CH₂CH₃)₂, —(CH₂)₄—N(CH₂—CH₃)₂, —(CH₂)₅—N(CH₂CH₃)₂, —(CH₂)₆—N(CH₂CH₃)₂, —CH₂—N(CH₃)₃⁺A⁻, —(CH₂)₂—N(CH₃)₃⁺A⁻, —(CH₂)₃—N(CH₃)₃⁺A⁻, —(CH₂)₄—N(CH₃)₃⁺A⁻, —(CH₂)₆—N(CH₃)₃⁺A⁻, —(CH₂)₆—N(CH₃)₃⁺A⁻, —CH₂—N(CH₂—CH₃)₃⁺A⁻, —(CH₂)₂—N(CH₂CH₃)₃⁺A⁻, —(CH₂)₃—N(CH₂CH₃)₃⁺A⁻, —(CH₂)₄—N(CH₂CH₃)₃⁺A⁻, —(CH₂)₆—N(CH₂—CH₃)₃⁺A⁻, —(CH₂)₆—N(CH₂CH₃)₃⁺A⁻, —CH₂—P(═O)(OH)₂, —(CH₂)₂—P(═O)(OH)₂, —(CH₂)₃—P(═O)(OH)₂, —(CH₂)₄—P(═O)(OH)₂, —(CH₂)₅—P(═O)(OH)₂, —(CH₂)₆—P(═O)(OH)₂; where PC stands for an organic or inorganic anion, which is preferably selected from the group consisting of Cl⁻, Br⁻, I⁻, dihydrogen phosphate, hydrogen phosphate, phosphate, bicarbonate, carbonate, acetate, lactate and citrate or for other physiologically tolerable anions; and the radicals may optionally be further substituted with hybrid radicals.

For the purposes of this invention, the term “linker” stands for the monomers derived from crosslinking agents, which are contained in the inventive copolymers and which contain at least two covalent bonds to at least two cyclodextrin monomers.

In the sense of this invention, the phrase “linker derived from crosslinking agents” stands for structural elements, which are contained in the inventive copolymers and whose chemical structure can be attributed to the structural modification of the crosslinking agents by the crosslinking reaction with cyclodextrin compounds (cf. in this regard also the exemplary reaction given above).

The linkers are preferably derived from bi- and/or polyfunctional crosslinking agents, preferably from homobifunctional, homopolyfunctional, heterobifunctional and/or heteropolyfunctional crosslinking agents and in particular from homo- and/or heterobifunctional crosslinking agents.

In a preferred embodiment, the linkers are derived from crosslinking agents selected from the group consisting of glyoxal, 1,2-dihaloethane (e.g., 1,2-dihaloethane), 1,3-dihalopropane (e.g., 1,3-dichloropropane), halocarboxylic acid halide [for example, haloacetic acid halide (e.g., chloroacetic acid chloride) or halopropionic acid halide (e.g., chloropropionic acid chloride)], epichlorohydrin, 4-chloro-1,2-epoxybutane, 1,2,3,4-diepoxybutane, tetramethylene diisocyanate and hexamethylene diisocyanate; where halogen and halide, independently of one another, preferably stand for —Cl, —Br or —I, especially preferably for —Cl or —Br and in particular for —Cl.

In another preferred embodiment, the linker is derived from a crosslinking agent selected from the group consisting of glyoxal, epichlorohydrin, 4-chloro-1,2-epoxybutane and 1,2,3,4-diepoxybutane. Especially advantageous are epichlorohydrin, 4-chloro-1,2-epoxybutane and 1,2,3,4-diepoxybutane. The crosslinking agent epichlorohydrin is especially preferred.

The linkers are preferably selected from the group comprising ˜CH₂˜, ˜CH₂—CH₂˜, ˜CH(CH₃)˜, ˜CH₂—CH₂—CH₂˜, ˜CH(CH₂—CH₃)˜, ˜CH(CH₃)—CH₂˜, ˜C(CH₃)₂˜, ˜CH₂—CH₂—CH₂—CH₂˜, ˜CH(CH₂—CH₂—CH₃)˜, ˜C(CH₃)(CH₂CH₃)˜, ˜C(CH₃)₂—CH₂˜, ˜CH(CH₂CH₃)—CH₂˜, ˜CH(CH₃)—CH(CH₃)˜, ˜CH₂—CH₂—CH₂—CH₂—CH₂˜, ˜CH₂—CH₂—CH₂—CH₂—CH₂—CH₂˜, ˜C(═O)—CH₂˜, ˜C(═O)—CH₂—CH₂˜, ˜C(═O)—CH(CH₃)˜, ˜C(═O)—CH₂—CH₂—CH₂˜, ˜C(═O)—CH(CH₂—CH₃)˜, ˜C(═O)—CH(CH₃)—CH₂˜, ˜C(═O)—C(CH₃)₂˜, ˜C(═O)—CH₂—CH₂—CH₂—CH₂˜, ˜C(═O)—CH(CH₂—CH₂—CH₃)˜, ˜C(═O)—C(CH₃)(CH₂CH₃)˜, ˜C(═O)—C(CH₃)₂—CH₂˜, ˜C(═O)—CH(CH₂CH₃)—CH₂˜, ˜C(═O)—CH(CH₃)—CH(CH₃)˜, ˜C(═O)—CH₂—CH₂—CH₂—CH₂—CH₂˜, ˜C(═O)—CH₂—CH₂—CH₂—CH₂—CH₂—CH₂˜, ˜C(═O)—CH₂—C(═O)˜, ˜C(═O)—CH₂—CH₂—C(═O)˜, ˜C(═O)CH(CH₃)—C(═O)˜, ˜C(═O)—CH₂—CH₂—CH₂—C(═O)˜, ˜C(═O)—CH(CH₂—CH₃)—C(═O)˜, ˜C(═O)—CH(CH₃)—CH₂—C(═O)˜, ˜C(═O)—C(CH₃)₂—C(═O)˜, ˜C(═O)—CH₂—CH₂—CH₂—CH₂—C(═O)˜, ˜C(═O)—CH(CH₂—CH₂—CH₃)—C(═O)˜, ˜C(═O)—C(CH₃)(CH₂CH₃)—C(═O)˜, ˜C(═O)—C(CH₃)₂—CH₂—C(═O)˜, ˜C(═O)—CH(CH₂CH₃)—CH₂—C(═O)˜, ˜C(═O)—CH(CH₃)—CH(CH₃)—C(═O)˜, ˜C(═O)—CH₂—CH₂—CH₂—CH₂—CH₂—C(═O)˜, ˜C(═O)—CH₂—CH₂—CH₂—CH₂—CH₂—CH₂—C(═O)˜, ˜C(═O)—NH—CH₂—NH—C(═O)˜, ˜C(═O)—NH—(CH₂)₂—NH—C(═O)˜, ˜C(═O)—NH—CH(CH₃)—NH—C(═O)˜, ˜C(═O)—NH—CH₂—CH₂—CH₂—NH—C(═O)˜, ˜C(═O)—NH—CH(CH₂—CH₃)—NH—C(═O)˜, ˜C(═O)—NH—CH(CH₃)—CH₂—NH—C(═O)˜, ˜C(═O)—NH—C(CH₃)₂—NH—C(═O)˜, ˜C(═O)—NH—CH₂—CH₂—CH₂—CH₂—NH—C(═O)˜, ˜C(═O)—NH—CH(CH₂—CH₂—CH₃)—NH—C(═O)˜, ˜C(═O)—NH—C(CH₃)(CH₂CH₃)—NH—C(═O)˜, ˜C(═O)—NH—C(CH₃)₂—CH₂—NH—C(═O)˜, ˜C(═O)—NH—CH(CH₂CH₃)—CH₂—NH—C(═O)˜, ˜C(═O)—NH—CH(CH₃)—CH(CH₃)—NH—C(═O)˜, ˜C(═O)—NH—(CH₂)₅—NH—C(═O)˜, ˜C(═O)—NH—(CH₂)₆—NH—C(═O)˜, ˜CH₂—CH(OH)—CH₂˜, ˜CH(CH₂OH)—CH₂˜, ˜CH₂—CH(OH)—CH₂—CH₂˜, ˜CH(CH₂OH)—CH₂—CH₂˜, ˜CH₂—CH(OH)—CH(OH)—CH₂˜, ˜CH(CH₂OH)—CH(CH₂OH)˜, ˜CH₂—CH(OH)—CH(CH₂OH)˜, ˜CH(OH)—CH₂—CH₂—CH₂˜ and ˜CH(CH₂OH)—CH₂—CH₂˜; where the symbol “˜” stands for covalent bond to a cyclodextrin monomer, and the linkers may optionally be further substituted with hybrid radicals.

In another preferred embodiment, the linkers are selected from the group comprising ˜CH₂˜, ˜CH₂—CH₂˜, ˜CH₂—CH₂—CH₂˜, ˜CH₂—CH₂—CH₂—CH₂˜, ˜CH₂—CH₂—CH₂—CH₂—CH₂˜, ˜CH₂—CH₂—CH₂—CH₂—CH₂—CH₂˜, ˜C(═O)—CH₂˜, ˜C(═O)—CH₂—CH₂˜, ˜C(═O)—CH₂—CH₂—CH₂˜, ˜C(═O)—CH₂—CH₂—CH₂—CH₂˜, ˜C(═O)—CH₂—CH₂—CH₂—CH₂—CH₂˜, ˜C(═O)—CH₂—CH₂—CH₂—CH₂—CH₂—CH₂˜, ˜C(═O)—CH₂—C(═O)˜, ˜C(═O)—CH₂—CH₂—C(═O)˜, ˜C(═O)—CH₂—CH₂—CH₂—C(═O)˜, ˜C(═O)—CH₂—CH₂—CH₂—CH₂—C(═O)˜, ˜C(═O)—CH₂—CH₂—CH₂—CH₂—CH₂—C(═O)˜, ˜C(═O)—CH₂—CH₂—CH₂—CH₂—CH₂—CH₂—C(═O)˜, ˜C(═O)—NH—CH₂—NH—C(═O)˜, ˜C(═O)—NH—CH₂—CH₂—NH—C(═O)˜, ˜C(═O)—NH—CH₂—CH₂—CH₂—NH—C(═O)˜, ˜C(═O)—NH—CH₂—CH₂—CH₂—CH₂—NH—C(═O)˜, ˜C(═O)—NH—CH₂—CH₂—CH₂—CH₂—CH₂—NH—C(═O)˜, ˜C(═O)—NH—CH₂—CH₂—CH₂—CH₂—CH₂—CH₂—NH—C(═O)˜, ˜CH₂—CH(OH)—CH₂˜, ˜CH(CH₂OH)—CH₂˜, ˜CH₂—CH(OH)—CH₂—CH₂˜, ˜CH(CH₂OH)—CH₂—CH₂˜, ˜CH₂—CH(OH)—CH(OH)—CH₂˜, ˜CH(CH₂OH)—CH(CH₂OH)˜ and ˜CH₂—CH(OH)—CH(CH₂OH)˜; where the symbol “˜” stands for a covalent bond to a cyclodextrin monomer, and the linkers may optionally be further substituted with hybrid radicals.

It is especially advantageous that the inventive cyclodextrin copolymers contain one or more linkers selected from the group comprising ˜C(═O)—NH—CH₂—CH₂—CH₂—CH₂—NH—C(═O)˜, ˜C(═O)—NH—CH₂—CH₂—CH₂—CH₂—CH₂—NH—C(═O)˜, ˜C(═O)—NH—CH₂—CH₂—CH₂—CH₂—CH₂—CH₂—NH—C(═O)˜, ˜CH₂—CH(OH)—CH₂˜, ˜CH(CH₂OH)—CH₂—, —CH₂—CH(OH)—CH₂—CH₂˜, ˜CH(CH₂OH)—CH₂—CH₂—, —CH₂—CH(OH)—CH(OH)—CH₂˜, ˜CH(CH₂OH)—CH(CH₂OH)˜, ˜CH₂—CH(OH)—CH(CH₂OH)˜; where the symbol “˜” stands for covalent bond to a cyclodextrin monomer, and the linkers may optionally be further substituted with hybrid radicals.

Linkers and substituents containing at least one nucleophilic radical (e.g., —OH, —SH, —NH₂), may react with the crosslinking agents and/or functionalizing agents used during the synthesis of the inventive copolymers. Through these reactions, structurally different and structurally complex linkers and substituents may be formed; they cannot be represented unambiguously with structural formulas because of their structural complexity. For example, in the reaction of the functionalizing agent 2-methyloxirane with the substituents —CH₂—CH(OH)—CH₃, the substituents —CH₂CH[O—CH₂—CH(OH)—CH₃]—CH₃ and —CH₂—CH[O—CH(CH₃)—CH₂—OH]—CH₂—OH may be formed, and these in turn may react further with 2-methyloxirane or other functionalizing agents or crosslinking agents to form various structurally complex substituents.

For the purposes of this invention, the term “hybrid radical” includes radicals, which are to be attributed to one or more reactions of substituents or linkers with cross-linking agents and/or functionalizing agents. The linkers and the substituents contained in the cyclodextrin monomers may be monosubstituted or polysubstituted by one or more hybrid radicals.

In a preferred embodiment, the hybrid radical is derived from:

at least one crosslinking agent selected from the group of 1,2-dihaloethane (e.g., 1,2-dichloroethane), 1,3-dihalopropane (e.g., 1,3-dichloropropane), halocarboxylic acid halide, [for example, haloacetic acid halide (e.g., chloroacetic acid chloride) or halopropionic acid halide (e.g., chloropropionic acid chloride)], epichlorohydrin, 4-chloro-1,2-epoxybutane, 1,2,3,4-diepoxybutane, tetramethylene diisocyanate and hexamethylene diisocyanate; where halogen and halide, independently of one another, preferably stand for —Cl, —Br or —I, especially preferably stand —Cl or —Br and in particular —Cl; and/or at least one functionalizing agent selected from the group of C₁₋₆-alkyl-Y, C₁₋₆-alkyl-C(═O)—Y, C₃₋₇-cycloalkyl-C(═O)—Y, Y—C₁₋₆-alkyl-C(═O)—OH, Y—C_{3-w}-cycloalkyl-C(═O)—OH, Y—C₁₋₆-alkyl-C(═O)—OC₁₋₆-alkyl, Y—C₃₋₁₀-cycloalkyl-C(═O)—O—C₃₋₁₀-cycloalkyl, [C₁₋₆-alkyl-C(═O)]₂O, [C₃₋₇-cycloalkyl-C(═O)]₂O, dihydrofuran-2,5-dione, furan-2,5-dione, dihydro-2H-pyran-2,6(3H)-dione, oxirane, (C₁₋₆-alkyl)oxirane, (C₃₋₇-cycloalkyl)oxirane, (di-C₁₋₆-alkyl)oxirane, (di-C₃₋₇-cycloalkyl)oxirane, 2-chloroethanol, di-C₁₋₆-alkyl sulfate, di-C₃₋₇-cycloalkyl sulfate, di-C₁₋₆-alkylphosphorochloridate, di-C₃₋₇-cycloalkylphosphorochloridate, NH₃, (C₁₋₆-alkyl)NH₂, (C₃₋₇-cycloalkyl)NH₂, (C₁₋₆-alkyl)₂NH, (C₃₋₇-cycloalkyl)₂NH, (C₁₋₆-alkyl)₃N, (C₃₋₇-cycloalkyl)₃N, [(Y—C₁₋₆-alkyl) N(C₁₋₆-alkyl)₃]⁺A⁻, 1,3-propane sultone and 1,4-butane sultone; where the C₁₋₆-alkyl radicals and the C₃₋₇-cycloalkyl radicals, independently of one another, may be unsubstituted or mono- or polysubstituted with the same or different Z radicals; where the additional substituents Z result in little or no significant crosslinking of the cyclodextrin monomers; “halide” preferably stands for chloride, bromide or iodide, especially preferably for chloride or bromide and in particular for chloride; and the Y radicals stand for leaving groups consisting of —Cl, —Br, —I, -mesyl, -tosyl and epoxy; especially preferably selected from the group consisting of —Cl, —Br, —I, -mesyl and -tosyl; and in particular —Cl; and the substituents Z independently of one another are preferably selected from the group consisting of —OH, ═O, —C(═O)OH, —C(═O)OCH₃, —C(═O)OCH₂CH₃, —NH₂, —NH(CH₃), —N(CH₃)₂, —N(CH₃)₃⁺A⁻, —NH(CH₂CH₃), —N(CH₂CH₃)₂ and —N(CH₂CH₃)₃⁺A⁻; especially preferably from the group consisting of —OH, ═O, —C(═O)OH, —C(═O)OCH₃ and —C(═O)OCH₂CH₃; and in particular selected from the group consisting of —OH, ═O and —C(═O)OH; where A⁻ stands for an organic or inorganic anion, which is preferably selected from the group consisting of Cl⁻, Br⁻, I⁻, dihydrogen phosphate, hydrogen phosphate, phosphate, bicarbonate, carbonate, acetate, lactate and citrate or for other physiologically tolerable anions.

In another preferred embodiment, the hybrid radical contains at least one structural element selected from the group consisting of ˜CH₂—CH₂—O˜, ˜CH₂—CH(O˜)—CH₃, ˜CH₂—CH(O˜)—CH₂—CH₃, ˜CH(CH₃)—CH₂—O˜, ˜CH(CH₂CH₃)—CH₂—O˜, ˜CH₂—CH(O˜)—CH₂—O˜, ˜CH(CH₂—O˜)—CH₂—O˜, ˜CH₂—CH(O˜)—C(═O)O˜, ˜CH[C(═O)O˜]—CH₂—O˜, ˜CH[C(═O)O—]—CH₂—O˜, —CH[C(═O)O˜]—CH₂—O˜, ˜CH₂—CH(O˜)—CH₂—O˜, ˜CH(CH₂O˜)—CH₂O˜, ˜CH₂—CH(O˜)—CH₂—CH₂O˜, ˜CH(CH₂O˜)—CH₂—CH₂O˜, ˜CH₂—CH(O˜)—CH(O˜)—CH₂O˜, ˜CH(CH₂O˜)—CH(CH₂O˜)—O˜, ˜CH₂—CH(O˜)—CH(CH₂O˜)—O˜, ˜CH(O˜)—CH₂—CH₂—CH₂O˜, ˜CH(CH₂O˜)—CH₂—CH₂O˜; where the symbol “˜” stands for a covalent single bond.

In another preferred embodiment, the hybrid radical contains at least one structural element selected from the group consisting of ˜CH₂—CH₂—O#, ˜CH₂—CH(O#)—CH₃, ˜CH₂—CH(O#)—CH₂—CH₃, ˜CH(CH₃)—CH₂—O#, ˜CH(CH₂CH₃)—CH₂—O#, ˜CH₂—CH(O#)—CH₂—O#, ˜CH(CH₂—O#)—CH₂—O#, ˜CH₂—CH(O#)—C(═O)O#, ˜CH[C(═O)O#]—CH₂—O#, ˜CH[C(═O)O#]—CH₂—O#, ˜CH[C(═O)O#]—CH₂—O#, ˜CH₂—CH(O#)—CH₂—O#, ˜CH(CH₂O#)—CH₂O#, ˜CH₂—CH(O#)—CH₂—CH₂O#, ˜CH(CH₂O#)—CH₂—CH₂O#, ˜CH₂—CH(O#)—CH(O#)—CH₂O#, ˜CH(CH₂O#)—CH(CH₂O#)—O#, ˜CH₂—C H(O#)—CH(CH₂O#)—O#, ˜CH(O#)—CH₂—CH₂—CH₂O#, ˜CH(CH₂O#)—CH₂—CH₂O#; where the symbol “˜” stands for a covalent single bond to an N—, O— or S atom, and the symbol “#” stands for a covalent bond to an H— or C atom.

In another preferred embodiment, the hybrid radical contains at least one structural element selected from the group consisting of §O—CH₂—CH₂—O#, §O—CH₂—CH(O#)—CH₃, §O—CH₂—CH(O#)—CH₂—CH₃, §O—CH(CH₃)—CH₂—O#, §O—CH(CH₂CH₃)—CH₂—O#, §O—CH₂—CH(O#)—CH₂—O#, §O—CH(CH₂—O#)—CH₂—O#, §O—CH₂—CH(O#)—C(═O)O#, §O—CH[C(═O)O#]—CH₂—O#, §O—CH[C(═O)O#]—CH₂—O#, §O—CH[C(═O)O#]—CH₂—O#, §O —CH₂—CH(O#)—CH₂—O#, §O—CH(CH₂O#)—CH₂O#, §O—CH₂—CH(O#)—CH₂—CH₂O#, §O—CH(CH₂O#)—CH₂—CH₂O#, §O—CH₂—CH(O#)—CH(O#)—CH₂O#, §O—CH(CH₂O#)—CH(CH₂O#)—O#, §O—CH₂—CH(O#)—CH(CH₂O#)—O#, §O—CH(O#)—CH₂—CH₂—CH₂O#, §O—CH(CH₂O#)—CH₂—CH₂O#; where the symbol “#” stands for —H or —CH₂§, and the symbol “§” stands for a covalent single bond to a C atom.

In another preferred embodiment, the hybrid radical contains at least one structural element selected from the group consisting of §O—CH₂—CH₂—OH, §O—CH₂—CH(OH)—CH₃, §O—CH₂—CH(OH)—CH₂—CH₃, §O—CH(CH₃)—CH₂—OH, §O—CH(CH₂—CH₃)—CH₂—OH, §O—CH₂—CH(OH)—CH₂—OH, §O—CH(CH₂—OH)—CH₂—OH, §O—CH₂—CH(OH)—C(═O)OH, §O—CH₂—CH(OH)—C(═O)O—CH₃, §O—CH₂—CH(OH)—C(═O)O—CH₂—CH₃, §O—CH[C(═O)OH]—CH₂—OH, §O—CH[C(═O)O—CH₃]—CH₂—OH, §O—CH[C(═O)O—CH₂—CH₃]—CH₂—OH, §O—CH₂—CH(OH)—CH₂—OH, §O—CH(CH₂OH)—CH₂OH, §O—CH₂—CH(OH)—CH₂—CH₂OH, §O—CH(CH₂OH)—CH₂—CH₂OH, §O—CH₂—CH(OH)—CH(OH)—CH₂OH, §O—CH(CH₂OH)—CH(CH₂OH)—OH, §O—CH₂—CH(OH)—CH(CH₂OH)—OH, §O—CH(OH)—CH₂—CH₂—CH₂OH and §O—CH(CH₂—OH)—CH₂—CH₂OH; where the symbol “§” stands for a covalent single bond to a C atom.

In an especially preferred embodiment, the inventive copolymer is a copolymer from the group consisting of:

(glucosyl)-cyclodextrin copolymer; (galactosyl)-cyclodextrin copolymer; (maltosyl)cyclodextrin copolymer; (maltriosyl)-cyclodextrin copolymer; (acetyl)-cyclodextrin copolymer; (propionyl)-cyclodextrin copolymer; (isobutyryl)-cyclodextrin copolymer; (butyryl)-cyclodextrin copolymer; (pivaloyl)-cyclodextrin copolymer; (pentanoyl)-cyclodextrin copolymer; (hexanoyl)-cyclodextrin copolymer; (cyclopropanecarbonyl)-cyclodextrin copolymer; (benzoyl)-cyclodextrin copolymer; (2-phenylacetyl)-cyclodextrin copolymer; (2-carboxyacetyl)-cyclodextrin copolymer; (3-carboxypropanoyl)-cyclodextrin copolymer; (4-carboxybutanoyl)-cyclodextrin copolymer; (2-methoxy-2-oxoacetyl)-cyclodextrin copolymer; (3-methoxy-3-oxopropanoyl)-cyclodextrin copolymer; (4-methoxy-4-oxobutanoyl)-cyclodextrin copolymer; (2-hydroxyacetyl)-cyclodextrin copolymer; (2-hydroxypropanoyl)-cyclodextrin copolymer; (3-hydroxypropanoyl)cyclodextrin copolymer; (2-hydroxybutanoyl)-cyclodextrin copolymer; (3-hydroxybutanoyl)-cyclodextrin copolymer; (4-hydroxybutanoyl)-cyclodextrin copolymer; (2-hydroxypentanoyl)-cyclodextrin copolymer; (3-hydroxypentanoyl)-cyclodextrin copolymer; (4-hydroxypentanoyl)-cyclodextrin copolymer; (5-hydroxypentanoyl)-cyclodextrin copolymer; (2,3-dihydroxypropanoyl)-cyclodextrin copolymer; (2,3-dihydroxybutanoyl)-cyclodextrin copolymer; (2,4-dihydroxybutanoyl)-cyclodextrin copolymer; (3,4-dihydroxybutanoyl)-cyclodextrin copolymer; (2,3-dihydroxypentanoyl)-cyclodextrin copolymer; (2,4-dihydroxypentanoyl)-cyclodextrin copolymer; (2.5-dihydroxypentanoyl)-cyclodextrin copolymer; (3,4-dihydroxypentanoyl)-cyclodextrin copolymer; (3,5-dihydroxypentanoyl)-cyclodextrin copolymer; (4,5-dihydroxypentanoyl)cyclodextrin copolymer; (2,3,4-trihydroxybutanoyl)-cyclodextrin copolymer; (2,3,4-trihydroxypentanoyl)-cyclodextrin copolymer; (2,3,5-trihydroxypentanoyl)-cyclodextrin copolymer; (2,4,5-trihydroxypentanoyl)-cyclodextrin copolymer; (3,4,5-trihydroxypentanoyl)-cyclodextrin copolymer; (2,3,4,5-tetrahydroxypentanoyl)-cyclodextrin copolymer; (2-aminoacetyl)-cyclodextrin copolymer; (2-aminopropanoyl)-cyclodextrin copolymer; (2-aminobutanoyl)-cyclodextrin copolymer; [2-(1H-imidazol-1-yl)acetyl]-cyclodextrin copolymer; [3-(1H-imidazol-1-yl)propanoyl]-cyclodextrin copolymer; [4-(1H-imidazol-1-yl)butanoyl]-cyclodextrin copolymer; [2-(1H-imidazol-1-yl)acetyl]-cyclodextrin copolymer; [3-(1H-imidazol-1-yl)propanoyl]-cyclodextrin copolymer; [4-(1H-imidazol-1-yl)butanoyl]-cyclodextrin copolymer; [2-(pyridin-3-yl)acetyl]-cyclodextrin copolymer; [3-(pyridin-3-yl)propanoyl]-cyclodextrin copolymer; [4-(pyridin-3-yl)butanoyl]-cyclodextrin copolymer; (methyl)-cyclodextrin copolymer; (ethyl)-cyclodextrin copolymer; (isopropyl)-cyclodextrin copolymer; (N-propyl)cyclodextrin copolymer; (isobutyl)-cyclodextrin copolymer; (N-butyl)-cyclodextrin copolymer; (sec-butyl)-cyclodextrin copolymer; (tert-butyl)-cyclodextrin copolymer; (cyclopropyl)-cyclodextrin copolymer; (phenyl)-cyclodextrin copolymer; (benzyl)cyclodextrin copolymer; (phenethyl)-cyclodextrin copolymer; (carboxymethyl)cyclodextrin copolymer; (1-carboxyethyl)-cyclodextrin copolymer; (2-carboxyethyl)cyclodextrin copolymer; (1-carboxypropyl)-cyclodextrin copolymer; (2-carboxypropyl)cyclodextrin copolymer; (3-carboxypropyl)-cyclodextrin copolymer; (1-carboxypropan-2-yl)-cyclodextrin copolymer; (4-carboxybutyl)-cyclodextrin copolymer; (5-carboxypentyl)-cyclodextrin copolymer; (5-carboxypentyl)-cyclodextrin copolymer; (2-methoxy-2-oxoethyl)-cyclodextrin copolymer; (3-methoxy-2-oxopropyl)-cyclodextrin copolymer; (4-methoxy-4-oxobutyl)-cyclodextrin copolymer; (5-methoxy-5-oxopentyl)cyclodextrin copolymer; (6-methoxy-6-oxohexyl)-cyclodextrin copolymer; (2-ethoxy-2-oxoethyl)-cyclodextrin copolymer; (3-ethoxy-2-oxopropyl)-cyclodextrin copolymer; (4-ethoxy-4-oxobutyl)-cyclodextrin copolymer; (5-ethoxy-5-oxopentyl)-cyclodextrin copolymer; (6-ethoxy-6-oxohexyl)-cyclodextrin copolymer; (3-carboxy-2-hydroxypropyl)-cyclodextrin copolymer; (2-hydroxy-4-methoxy-4-oxobutyl)-cyclodextrin copolymer; (4-ethoxy-2-hydroxy-4-oxobutyl)-cyclodextrin copolymer; (1-carboxy-2-hydroxyethyl)-cyclodextrin copolymer; (3-hydroxy-1-methoxy-1-oxopropan-2-yl)cyclodextrin copolymer; (1-ethoxy-3-hydroxy-1-oxopropan-2-yl)-cyclodextrin copolymer; (1-carboxy-3-hydroxypropan-2-yl)-cyclodextrin copolymer; (1-hydroxy-4-methoxy-4-oxobutan-2-yl)-cyclodextrin copolymer; (4-ethoxy-1-hydroxy-4-oxobutan-2-yl)cyclodextrin copolymer; (hydroxymethyl)-cyclodextrin copolymer; (1-hydroxyethyl)cyclodextrin copolymer; (2-hydroxyethyl)-cyclodextrin copolymer; (1-hydroxypropyl)cyclodextrin copolymer; (2-hydroxypropyl)-cyclodextrin copolymer; (1-hydroxypropan-2-yl)-cyclodextrin copolymer; (2-hydroxypropan-2-yl)-cyclodextrin copolymer; (1-hydroxybutyl)-cyclodextrin copolymer; (2-hydroxybutyl)-cyclodextrin copolymer; (3-hydroxybutyl)-cyclodextrin copolymer; (4-hydroxybutyl)-cyclodextrin copolymer; (1-hydroxypentyl)-cyclodextrin copolymer; (2-hydroxypentyl)-cyclodextrin copolymer; (3-hydroxypentyl)-cyclodextrin copolymer; (4-hydroxypentyl)-cyclodextrin copolymer; (5-hydroxypentyl)-cyclodextrin copolymer; (1,2-dihydroxyethyl)-cyclodextrin copolymer; (1,2-dihydroxypropyl)-cyclodextrin copolymer; (1,3-dihydroxypropyl)-cyclodextrin copolymer; (2,3-dihydroxypropyl)-cyclodextrin copolymer; (1,2-dihydroxybutyl)cyclodextrin copolymer; (1,3-dihydroxybutyl)-cyclodextrin copolymer; (1,4-dihydroxybutyl)-cyclodextrin copolymer; (2,3-dihydroxybutyl)-cyclodextrin copolymer; (2,4-dihydroxybutyl)-cyclodextrin copolymer; (3,4-dihydroxybutyl)-cyclodextrin copolymer; (1,2-dihydroxypentyl)-cyclodextrin copolymer; (1,3-dihydroxypentyl)-cyclodextrin copolymer; (1,4-dihydroxypentyl)-cyclodextrin copolymer; (1,5-dihydroxypentyl)cyclodextrin copolymer; (2,3-dihydroxypentyl)-cyclodextrin copolymer; (2,4-dihydroxypentyl)-cyclodextrin copolymer; (2.5-dihydroxypentyl)-cyclodextrin copolymer; (3,4-dihydroxypentyl)-cyclodextrin copolymer; (3,5-dihydroxypentyl)-cyclodextrin copolymer; (4,5-dihydroxypentyl)-cyclodextrin copolymer; (1,2,3-trihydroxypropyl)-cyclodextrin copolymer; (1,2,3-trihydroxybutyl)-cyclodextrin copolymer; (1,2,4-trihydroxybutyl)-cyclodextrin copolymer; (1,3,4-trihydroxybutyl)-cyclodextrin copolymer; (2,3,4-trihydroxybutyl)-cyclodextrin copolymer; (1,2,3-trihydroxypentyl)-cyclodextrin copolymer; (1,2,4-trihydroxypentyl)-cyclodextrin copolymer; (1,2,5-trihydroxypentyl)cyclodextrin copolymer; (1,3,4-trihydroxypentyl)-cyclodextrin copolymer; (1,3,5-trihydroxypentyl)-cyclodextrin copolymer; (1,4,5-trihydroxypentyl)-cyclodextrin copolymer; (2,3,4-trihydroxypentyl)-cyclodextrin copolymer; (2,3,5-trihydroxypentyl)-cyclodextrin copolymer; (2,4,5-trihydroxypentyl)-cyclodextrin copolymer; (3,4,5-trihydroxypentyl)-cyclodextrin copolymer; (1,2,3,4-tetrahydroxybutyl)-cyclodextrin copolymer; (1,2,3,4-tetrahydroxypentyl)-cyclodextrin copolymer; (1,2,4,5-tetrahydroxypentyl)cyclodextrin copolymer; (2,3,4,5-tetrahydroxypentyl)-cyclodextrin copolymer; (1,2,3,4,5-pentahydroxypentyl)-cyclodextrin copolymer; [(sulfoxy)methyl]-cyclodextrin copolymer; [2-(sulfoxy)ethyl]-cyclodextrin copolymer; [3-(sulfoxy)propyl]-cyclodextrin copolymer; [4-(sulfoxy)butyl]-cyclodextrin copolymer; [(phosphonooxy)methyl]-cyclodextrin copolymer; [2-(phosphonooxy)ethyl]-cyclodextrin copolymer; [3-(phosphonooxy)propyl]-cyclodextrin copolymer; [4-(phosphonooxy)butyl]-cyclodextrin copolymer; [4-(phosphonooxy)butyl]-cyclodextrin copolymer; (sulfomethyl)-cyclodextrin copolymer; (2-sulfoethyl)-cyclodextrin copolymer; (3-sulfopropyl)-cyclodextrin copolymer; (4-sulfobutyl)-cyclodextrin copolymer; (5-sulfopentyl)-cyclodextrin copolymer; (6-sulfohexyl)-cyclodextrin copolymer; (phosphonomethyl)-cyclodextrin copolymer; (2-phosphonoethyl)-cyclodextrin copolymer; (3-phosphonopropyl)-cyclodextrin copolymer; (4-phosphonobutyl)-cyclodextrin copolymer; (5-phosphonopentyl)-cyclodextrin copolymer; (6-phosphonohexyl)-cyclodextrin copolymer; (2-phosphonovinyl)cyclodextrin copolymer; (3-phosphonoallyl)-cyclodextrin copolymer; (4-phosphonobut-3-enyl)-cyclodextrin copolymer; (5-phosphonopent-4-enyl)-cyclodextrin copolymer; (6-phosphonohex-5-enyl)-cyclodextrin copolymer; (aminomethyl)-cyclodextrin copolymer; (2-aminoethyl)-cyclodextrin copolymer; (3-aminopropyl)-cyclodextrin copolymer; (4-aminobutyl)-cyclodextrin copolymer; (5-aminopentyl)-cyclodextrin copolymer; (6-aminohexyl)-cyclodextrin copolymer; [(N,N-dimethylamino)methyl]-cyclodextrin copolymer; [2-(N,N-dimethylamino)ethyl]-cyclodextrin copolymer; [3-(N,N-dimethylamino)propyl]-cyclodextrin copolymer; [4-(N,N-dimethylamino)butyl]-cyclodextrin copolymer; [5-(N,N-dimethylamino)pentyl]-cyclodextrin copolymer; [6-(N,N-dimethylamino)hexyl]-cyclodextrin copolymer; [(N,N-diethylamino)methyl]-cyclodextrin copolymer; [2-(N,N-diethylamino)ethyl]-cyclodextrin copolymer; [3-(N,N-diethylamino)propyl]-cyclodextrin copolymer; [4-(N,N-diethylamino)butyl]-cyclodextrin copolymer; [5-(N,N-diethylamino)pentyl]-cyclodextrin copolymer; [6-(N,N-diethylamino)hexyl]-cyclodextrin copolymer; [(trimethylammonio)methyl]-cyclodextrin copolymer chloride; [2-(trimethylammonio)ethyl]-cyclodextrin copolymer chloride; [3-(trimethylammonio)propyl]-cyclodextrin copolymer chloride; [4-(trimethylammonio)butyl]-cyclodextrin copolymer chloride; [5-(trimethylammonio) pentyl]-cyclodextrin copolymer chloride; [6-(trimethylammonio)hexyl]-cyclodextrin copolymer chloride; [(triethylammonio)methyl]-cyclodextrin copolymer chloride; [2-(triethylammonio)ethyl]-cyclodextrin copolymer chloride; [3-(triethylammonio)propyl]-cyclodextrin copolymer chloride; [4-(triethylammonio)butyl]-cyclodextrin copolymer chloride; [5-(triethylammonio)pentyl]-cyclodextrin copolymer chloride; [6-(triethylammonio)hexyl]-cyclodextrin copolymer chloride; [(1H-imidazol-1-yl)methyl]-cyclodextrin copolymer; [2-(1H-imidazol-1-yl)ethyl]-cyclodextrin copolymer; [3-(1H-imidazol-1-yl)propyl]-cyclodextrin copolymer; [4-(1H-imidazol-1-yl)butyl]-cyclodextrin copolymer; (pyridin-3-ylmethyl)cyclodextrin copolymer; [2-(pyridin-3-yl)ethyl]-cyclodextrin copolymer; [3-(pyridin-3-yl)propyl]-cyclodextrin copolymer; [4-(pyridin-3-yl)butyl]-cyclodextrin copolymer; [(β-D-glucopyranosyloxyuronic acid) methyl]-cyclodextrin copolymer; [2-(β-D-glucopyranosyloxyuronic acid) ethyl]-cyclodextrin copolymer; [3-(β-D-glucopyranosyloxyuronic acid)propyl]-cyclodextrin copolymer; [4-(β-D-glucopyranosyloxyuronic acid) butyl]-cyclodextrin copolymer; [5-(β-D-glucopyranosyloxyuronic acid)pentyl]-cyclodextrin copolymer and [6-(β-D-glucopyranosyloxyuronic acid) hexyl]-cyclodextrin copolymer; where the term “cyclodextrin copolymer” stands for α-, β-, γ-, α/β-, α/γ-, β/γ- or α/β/γ-cyclodextrin copolymer, preferably for α-, β- or γ-cyclodextrin copolymer, more preferably for β-cyclodextrin copolymer, and in particular for α-cyclodextrin-epichlorohydrin copolymer, β-cyclodextrin-epichlorohydrin copolymer, γ-cyclodextrin-epichlorohydrin copolymer, α-cyclodextrin-4-chloro-1,2-epoxybutane copolymer, β-cyclodextrin-4-chloro-1,2-epoxybutane copolymer, γ-cyclodextrin-4-chloro-1,2-epoxybutane copolymer, α-cyclodextrin-1,2,3,4-diepoxybutane copolymer, β-cyclodextrin-1,2,3,4-diepoxybutane copolymer, γ-cyclodextrin-1,2,3,4-diepoxybutane copolymer, α-cyclodextrin-tetramethylene diisocyanate copolymer, β-cyclodextrin-tetramethylene diisocyanate copolymer, γ-cyclodextrin-tetramethylene diisocyanate copolymer, α-cyclodextrin-hexamethylene diisocyanate copolymer, β-cyclodextrin-hexamethylene diisocyanate copolymer or γ-cyclodextrin-hexamethylene diisocyanate copolymer.

In an especially preferred embodiment, the inventive copolymer is a copolymer from the group consisting of:

(acetyl)-α-cyclodextrin-epichlorohydrin copolymer; (2-carboxyacetyl)-α-cyclodextrin-epichlorohydrin copolymer; (3-carboxypropanoyl)-α-cyclodextrin-epichlorohydrin copolymer; (2-hydroxyacetyl)-α-cyclodextrin-epichlorohydrin copolymer; (methyl)-α-cyclodextrin-epichlorohydrin copolymer; (ethyl)-α-cyclodextrin-epichlorohydrin copolymer; (carboxymethyl)-α-cyclodextrin-epichlorohydrin copolymer; (1-carboxyethyl)-α-cyclodextrin-epichlorohydrin copolymer; (2-carboxyethyl)-α-cyclodextrin-epichlorohydrin copolymer; (hydroxymethyl)-α-cyclodextrin-epichlorohydrin copolymer; (1-hydroxyethyl)-α-cyclodextrin-epichlorohydrin copolymer; (2-hydroxyethyl)-α-cyclodextrin-epichlorohydrin copolymer; (1-hydroxypropyl)-α-cyclodextrin-epichlorohydrin copolymer; (2-hydroxypropyl)-α-cyclodextrin-epichlorohydrin copolymer; (1-hydroxypropan-2-yl)-α-cyclodextrin-epichlorohydrin copolymer; (2-hydroxypropan-2-yl)-α-cyclodextrin-epichlorohydrin copolymer; [3-(sulfoxy)propyl]-α-cyclodextrin-epichlorohydrin copolymer; [4-(sulfoxy)butyl]-α-cyclodextrin-epichlorohydrin copolymer; (acetyl)-β-cyclodextrin-epichlorohydrin copolymer; (2-carboxyacetyl)-β-cyclodextrin-epichlorohydrin copolymer; (3-carboxypropanoyl)-β-cyclodextrin-epichlorohydrin copolymer; (2-hydroxyacetyl)-β-cyclodextrin-epichlorohydrin copolymer; (methyl)-β-cyclodextrin-epichlorohydrin copolymer; (ethyl)-β-cyclodextrin-epichlorohydrin copolymer; (carboxymethyl)-β-cyclodextrin-epichlorohydrin copolymer; (1-carboxyethyl)-β-cyclodextrin-epichlorohydrin copolymer; (2-carboxyethyl)-β-cyclodextrin-epichlorohydrin copolymer; (hydroxymethyl)-β-cyclodextrin-epichlorohydrin copolymer; (1-hydroxyethyl)-β-cyclodextrin-epichlorohydrin copolymer; (2-hydroxyethyl)-β-cyclodextrin-epichlorohydrin copolymer; (1-hydroxypropyl)-β-cyclodextrin-epichlorohydrin copolymer; (2-hydroxypropyl)-β-cyclodextrin-epichlorohydrin copolymer; (1-hydroxypropan-2-yl)-β-cyclodextrin-epichlorohydrin copolymer; (2-hydroxypropan-2-yl)-6-cyclodextrin-epichlorohydrin copolymer; [3-(sulfoxy)propyl]-β-cyclodextrin-epichlorohydrin copolymer; [4-(sulfoxy)butyl]-β-cyclodextrin-epichlorohydrin copolymer; (acetyl)-γ-cyclodextrin-epichlorohydrin copolymer; (2-carboxyacetyl)-γ-cyclodextrin-epichlorohydrin copolymer; (3-carboxypropanoyl)-γ-cyclodextrin-epichlorohydrin copolymer; (2-hydroxyacetyl)-γ-cyclodextrin-epichlorohydrin copolymer; (methyl)-γ-cyclodextrin-epichlorohydrin copolymer; (ethyl)-γ-cyclodextrin-epichlorohydrin copolymer; (carboxymethyl)-γ-cyclodextrin-epichlorohydrin copolymer; (1-carboxyethyl)-γ-cyclodextrin-epichlorohydrin copolymer; (2-carboxyethyl)-γ-cyclodextrin-epichlorohydrin copolymer; (hydroxymethyl)-γ-cyclodextrin-epichlorohydrin copolymer; (1-hydroxyethyl)-γ-cyclodextrin-epichlorohydrin copolymer; (2-hydroxyethyl)-γ-cyclodextrin-epichlorohydrin copolymer; (1-hydroxypropyl)-γ-cyclodextrin-epichlorohydrin copolymer; (2-hydroxypropyl)-γ-cyclodextrin-epichlorohydrin copolymer; (1-hydroxypropan-2-yl)-γ-cyclodextrin-epichlorohydrin copolymer; (2-hydroxypropan-2-yl)-γ-cyclodextrin-epichlorohydrin copolymer; [3-(sulfoxy)propyl]-γ-cyclodextrin-epichlorohydrin copolymer and [4-(sulfoxy)butyl]-γ-cyclodextrin-epichlorohydrin copolymer.

Another preferred subject matter of this invention relates to a process comprising the process step:
mixing at least one cyclodextrin compound with
   (a) at least one crosslinking agent or
   (b) at least one crosslinking agent and at least one functionalizing agent;
      for use in the treatment of diseases of the urogenital system.

In a preferred embodiment, the inventive copolymers can be obtained by a process comprising the process step:
mixing at least one α-, β- or γ-cyclodextrin compound with
   (a) at least one crosslinking agent or
   (b) at least one crosslinking agent and at least one functionalizing agent.

In another preferred embodiment, the inventive copolymers can be obtained by a process comprising the process step:
mixing an unsubstituted or monosubstituted or polysubstituted α-, β- or γ-cyclodextrin, with
   (a) a crosslinking agent or
   (b) a crosslinking agent and a functionalizing agent.

The “mixing” process step is preferably performed in the presence of a solvent or solvent mixture.

The cyclodextrin compound is preferably a compound of the general structure I

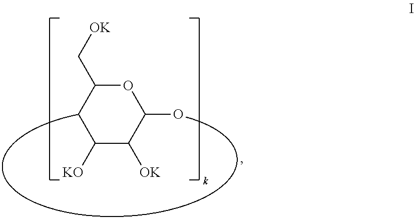

where
   k stands for 6, 7 or 8,
   each individual K radical stands either
      (a) for hydrogen; or
      (b) for glycosyl, —C(═O)R⁶ or R⁷; where
         R⁶ and R⁷, independently of one another, stand for C₁₋₆-alkyl, C₃₋₇-cycloalkyl, phenyl or 5- to 7-membered heteroaryl.

In the sense of this invention, the variable “k” defines the number of glucosyl units in the ring system of the cyclodextrin compounds: for α-cyclodextrin compounds, k=6; for β-cyclodextrin compounds, k=7; and for γ-cyclodextrin compounds, k=8.

The variable “k” preferably stands for 6 or 7 and in particular for 7.

In a preferred embodiment the total degree of substitution of the cyclodextrin compounds is 0<TDS_{b}<k×3, where TDS_{b} stands for the total degree of substitution of the compound of the general structure I with respect to the substituents (b), i.e., glycosyl, —C(═O)R⁶ and R⁷.

In the case when the cyclodextrin compounds are monosubstituted or polysubstituted with radicals having nucleophilic groups (e.g., —OH, —SH or primary, secondary, or tertiary amino), the total degree of substitution may be 0<TDS_{b}<k×3 as well as TDS_{b}≧k×3 because the nucleophilic groups may themselves be substituted.

With respect to the compounds of the general structure I, the total degree of substitution (TDS_{b}) with respect to the substituents (b) for a=6 is preferably 0<TDS_{b}<1.0 <TDS_{b}<2.0 <TDS_{b}<3 or 0<TDS_{b}<4; more preferably 0<TDS_{b}<5.0 <TDS_{b <}6.0 <TDS_{b}<7 or 0<TDS_{b}<8; even more preferably 0<TDS_{b}<9.0 <TDS_{b}<10.0 <TDS_{b}<11 or 0<TDS_{b}<12; most preferably 0<TDS_{b}<13.0 <TDS_{b}<14.0 <TDS_{b}<15 or 0<TDS_{b}<16; and in particular 0<TDS_{b}<17 or 0<TDS_{b}<18.

With respect to the compounds of the general structure I, the total degree of substitution (TDSb) with respect to the substituents (b) for a=7 is preferably 0 <TDS_{b}<1.0 <TDS_{b}<2.0 <TDS_{b}<3.0 <TDS_{b}<4 or 0<TDS_{b}<5; more preferably 0 <TDS_{b}<6.0 <TDS_{b}<7.0 <TDS_{b}<8.0 <TDS_{b}<9 or 0<TDS_{b}<10; even more preferably 0<TDS_{b}<11.0 <TDS_{b}<12.0 <TDS_{b}<13.0 <TDS_{b}<14 or 0<TDS_{b}<15; most preferably 0<TDS_{b}<16.0 <TDS_{b}<17.0 <TDS_{b}<18 or 0<TDS_{b}<19; and in particular 0<TDS_{b}<20 or 0<TDS_{b}<21.

With respect to the compounds of the general structure I, the total degree of substitution (TDSb) with respect to the substituents (b) for a=8 is preferably 0 <TDS_{b}<1.0 <TDS_{b}<2.0 <TDS_{b}<3.0 <TDS_{b}<4.0 <TDS_{b}<5 or 0<TDS_{b}<6; more preferably 0<TDS_{b}<7.0 <TDS_{b}<8.0 <TDS_{b}<9.0 <TDS_{b}<10.0 <TDS_{b}<11 or 0 <TDS_{b}<12; even more preferably 0<TDS_{b}<13.0 <TDS_{b}<14.0 <TDS_{b}<15.0 <TDS_{b}<16.0 <TDS_{b}<17 or 0<TDS_{b}<18; most preferably 0<TDS_{b}<19.0 <TDS_{b}<20.0 <TDS_{b}<21 or 0<TDS_{b}<22; and in particular 0<TDS_{b}<23 or 0<TDS_{b}<24.

The term “total degree of substitution” or TDS is understood in the sense of this invention to refer to the average number of substituents, which may be the same or different and are contained in a substituted cyclodextrin compound, where hydrogen is not included as a substituent (cf. J. Blanchard, S. Proniuk; Some Important Considerations in the Use of Cyclodextrins; Pharm. Res. 1999, 16(12), 1796-1798).

For example, the TDS in methylated β-cyclodextrin may assume values from >0 to ≦21, where a TDS of 21 means that all the hydroxyl groups of the β-cyclodextrin are methylated. A TDS of >0 to <1 means that a certain number of β-cyclodextrin molecules do not have a methyl substituent, and a TDS of 8.3, for example, means that each of the β-cyclodextrin molecules has an average of 8.3 methyl substituents. In the case of cyclodextrins with nucleophilic radicals in the substituents (e.g., the nucleophilic OH group in the hydroxypropyl substituent of the compound hydroxypropyl-β-cyclodextrin), the TDS may also be >21 because the nucleophilic groups of the hydroxypropyl substituents may themselves be substituted. These cases may thus yield TDS values which are greater than the number of maximum possible substituent sites on the cyclodextrin ring.

In a preferred embodiment, the glycosyl is a monosaccharide selected from the group consisting of mannitol, isomalt, lactitol, sorbitol, glucitol, xylitol, threitol, erythritol, arabitol, glyceraldehyde, erythrose, threose, ribose, arabinose, xylose, lyxose, allose, altrose, glucose, mannose, gulose, idose, galactose, talose, dihydroxyacetone, erythrulose, ribulose, xylulose, psicose, fructose, sorbose, tagatose, glucosamine, fucosamine, N-acetylmannosamine, hamamelose, apiose, gluconic acid, galacturonic acid, erythraric acid, ascorbic acid, glucuronic acid, abequose, apiose, cladinose, deoxyribose, deoxyglucose, digitalose, digitoxose, fucose, fucosamine, galactosamine, glucosamine, glucosaminitol, glycerol, glycerone, mannosamine, mannosaccharic acid, neuraminic acid, rhamnose, mucic acid, sedoheptulose, streptose, trehalosamine, trehalose, tartaric acid and glucaric acid. Glucose is especially advantageous.

The glycosyl may also be a disaccharide, trisaccharide, oligosaccharide or polysaccharide, preferably containing the same or different monomers of the monosaccharides listed above.

In a preferred embodiment, glycosyl is selected from the group consisting of mannitol, isomalt, lactitol, sorbitol, glucitol, xylitol, threitol, erythritol, arabitol, glyceraldehyde, erythrose, threose, ribose, arabinose, xylose, lyxose, allose, altrose, glucose, mannose, gulose, idose, galactose, talose, dihydroxyacetone, erythrulose, ribulose, xylulose, psicose, fructose, sorbose, tagatose, glucosamine, fucosamine, N-acetylmannosamine, hamamelose, apiose, gluconic acid, galacturonic acid, erythraric acid, ascorbic acid, glucuronic acid, abequose, apiose, cladinose, deoxyribose, deoxyglucose, digitalose, digitoxose, fucose, fucosamine, galactosamine, glucosamine, glucosaminitol, glycerol, glycerone, mannosamine, mannosaccharic acid, neuraminic acid, rhamnose, mucic acid, sedoheptulose, streptose, trehalosamine, trehalose, tartaric acid, glucaric acid, maltose and maltotriose. It is especially preferable for glycosyl to be selected from the group consisting of glucose, galactose, maltose and maltotriose.

With respect to the R⁶ and R⁷ radicals, C₁₋₆-alkyl is preferably selected from the group consisting of methyl, ethyl, isopropyl, N-propyl, isobutyl, N-butyl, sec-butyl, tert-butyl, N-pentyl, isopentyl, neopentyl, isohexyl, N-hexyl, neohexyl, 3-methylpentyl, 2,3-dimethylbutyl, ethenyl, ethynyl, prop-1-enyl, isobutenyl, N-butenyl, cis-2-butenyl, trans-2-butenyl, 1,2-butadienyl, 1,3-butadienyl, pent-1-enyl, cis-pent-2-enyl, trans-pent-2-enyl, 2-methylbut-1-enyl, 2-methylbut-2-enyl, 3-methylbut-1-enyl, hex-1-enyl, hex-2-enyl and hex-3-enyl. It is especially preferable for C₁₋₆-alkyl to be selected from the group consisting of methyl, ethyl, isopropyl, N-propyl, isobutyl, N-butyl, sec-butyl, tert-butyl, N-pentyl, isopentyl, neopentyl, isohexyl, N-hexyl, neohexyl, 3-methylpentyl, 2,3-dimethylbutyl, ethenyl, ethynyl and prop-1-enyl. The aforementioned C₁₋₆-alkyl radicals may be unsubstituted or mono- or polysubstituted (e.g., two, three, four or five times) with R⁸ radicals.

With respect to the R⁶ and R⁷ radicals, C₃₋₇-cycloalkyl is preferably selected from the group consisting of cyclopropyl, cyclobutyl, cyclopentyl, cyclohexyl, cycloheptyl, cyclopropenyl, cyclobutenyl, cyclopentenyl, cyclopentadienyl, cyclohexenyl, cyclohexadienyl, cycloheptenyl, cycloheptadienyl, cycloheptatrienyl, pyrrolinyl, pyrrolidinyl, tetrahydrofuranyl, pyrazolinyl, pyrazolidinyl, imidazolinyl, imidazolidinyl, dioxolanyl, piperidinyl, tetrahydropyranyl, piperazinyl, dioxanyl, morpholinyl, quinuclidinyl, pyrazolinyl, pyrazolinonyl, pyranyl, indolinyl, quinolizinyl, chromanyl, isochromanyl, chromenyl, isochromenyl and benzodioxolanyl. It is especially preferable for C₃₋₇-cycloalkyl to be selected from the group consisting of cyclopropyl, cyclobutyl, cyclopentyl, cyclohexyl, cycloheptyl, cyclopropenyl, cyclobutenyl, cyclopentenyl, pyrrolinyl, pyrrolidinyl, tetrahydrofuranyl, pyrazolinyl, pyrazolidinyl, imidazolinyl, imidazolidinyl, dioxolanyl, piperidinyl, tetrahydropyranyl, piperazinyl, dioxanyl, morpholinyl, quinuclidinyl, pyrazolinyl, pyrazolinonyl and pyranyl. It is also especially preferable for C₃₋₇-cycloalkyl to be selected from the group consisting of cyclopropyl, cyclobutyl, cyclopentyl, cyclohexyl, tetrahydrofuranyl, piperidinyl, piperazinyl, morpholinyl and quinuclidinyl. It is especially preferable for C₃₋₇-cycloalkyl to be selected from the group consisting of cyclopropyl, cyclobutyl, cyclopentyl, cyclohexyl, piperidinyl, piperazinyl and morpholinyl. The aforementioned C₃₋₇-cycloalkyl radicals may be unsubstituted or mono- or polysubstituted (e.g., two, three, four or five times) with R⁸ radicals. The C₃₋₇-cycloalkyl radical may also be bound to the cyclodextrin compounds via a C₁₋₄-alkylene bridge, preferably —CH₂—, —CH₂—CH₂—, —CH₂—CH₂—CH₂—, or —CH₂—CH₂—CH₂—CH₂—.

With respect to the R⁶ and R⁷ radicals, the phenyl radical may be unsubstituted or mono- or polysubstituted (e.g., two, three, four or five times) with R⁸ radicals. The phenyl radical may also be bound to the cyclodextrin monomers via a C₁₋₄-alkylene bridge, preferably —CH₂—, —CH₂—CH₂—, —CH₂—CH₂—CH₂—, or —CH₂—CH₂—CH₂—CH₂—.

With respect to the radicals R⁶ and R⁷, the 5- to 7-membered heteroaryl is preferably selected from the group consisting of pyrrolyl, pyrazolyl, imidazolyl, thienyl, furyl, oxazolyl, isoxazolyl, thiazolyl, isothiazolyl, furazanyl, oxadiazolyl, pyridyl, pyridazinyl, pyrazinyl, pyrimidinyl, indolyl, isoindolyl, indolizinyl, indazolyl, benzotriazolyl, quinolinyl, isoquinolinyl, naphthyridinyl, quinazolinyl, phthalazinyl, quinoxalinyl, purinyl, pteridinyl, benzofuryl, isobenzofuryl, benzothienyl, benzothiazolyl and benzothiadiazolyl. In a preferred embodiment, the 5- to 7-membered heteroaryl is selected from the group consisting of pyrrolyl, pyrazolyl, imidazolyl, thienyl, furyl, oxazolyl, isoxazolyl, thiazolyl, isothiazolyl, pyridyl, indolyl, quinolinyl, purinyl, pteridinyl and benzofuryl. In an especially preferred embodiment, the 5- to 7-membered heteroaryl is selected from the group consisting of pyrrolyl, pyrazolyl, imidazolyl, thienyl, furyl, oxazolyl, isoxazolyl, thiazolyl, isothiazolyl and pyridyl. The aforementioned 5- to 7-membered heteroaryl radicals may be unsubstituted or mono- or polysubstituted (e.g., two, three, four or five times) with R⁸ radicals. The 5- to 7-membered heteroaryl radical may also be bound to the cyclodextrin compounds via a C₁₋₄-alkylene bridge, preferably —CH₂—, —CH₂—CH₂—, —CH₂—CH₂—CH₂—, or —CH₂—CH₂—CH₂—CH₂—.

In a preferred embodiment, each individual K radical stands for either hydrogen or a radical selected from the group consisting of glucose, galactose, maltose, maltotriose, —C(═O)—C₁₋₆-alkyl, —C(═O)—C₃₋₇-cycloalkyl, —C(═O)-phenyl, —C(═O)-(5- to 7-membered heteroaryl), —C₁₋₆-alkyl, —C₃₋₇-cycloalkyl, phenyl and 5- to 7-membered heteroaryl; where C₁₋₆-alkyl, C₃₋₇-cycloalkyl, phenyl and 5- to 7-membered heteroaryl may be unsubstituted or mono- or polysubstituted (e.g., two, three, four or five times) with the same or different R⁸ radicals; and C₃₋₇-cycloalkyl, phenyl and 5- to 7-membered heteroaryl radical may be bound to the cyclodextrin compounds via a C₁₋₄-alkylene bridge, preferably —CH₂—, —CH₂—CH₂—, —CH₂—CH₂—CH₂—, or —CH₂—CH₂—CH₂—CH₂—.

In another preferred embodiment, each individual K radical stands either for hydrogen or for a radical selected from the group consisting of glucose, galactose, maltose, maltotriose, —C(═O)-methyl, —C(═O)-ethyl, —C(═O)-isopropyl, —C(═O)—N-propyl, —C(═O)-isobutyl, —C(═O)—N-butyl, —C(═O)-sec-butyl, —C(═O)-tert-butyl, —C(═O)—N-pentyl, —C(═O)-isopentyl, —C(═O)-neopentyl, —C(═O)-isohexyl, —C(═O)—N-hexyl, neohexyl, —C(═O)-3-methylpentyl, —C(═O)-2,3-dimethylbutyl, —C(═O)-ethenyl, —C(═O)-ethynyl, —C(═O)-prop-1-enyl, —C(═O)—C₃₋₇-cycloalkyl, —C(═O)-cyclopropyl, —C(═O)-cyclobutyl, —C(═O)-cyclopentyl, —C(═O)-cyclohexyl, —C(═O)-piperidinyl, —C(═O)-piperazinyl, —C(═O)-morpholinyl, —C(═O)-phenyl, —C(═O)-imidazolyl, —C(═O)-thienyl, —C(═O)-furyl, —C(═O)-oxazolyl, —C(═O)-isoxazolyl, —C(═O)-thiazolyl, —C(═O)-isothiazolyl, —C(═O)pyridyl, methyl, ethyl, isopropyl, N-propyl, isobutyl, N-butyl, sec-butyl, tert-butyl, N-pentyl, isopentyl, neopentyl, isohexyl, N-hexyl, neohexyl, 3-methylpentyl, 2,3-dimethylbutyl, ethenyl, ethynyl, prop-1-enyl, cyclopropyl, cyclobutyl, cyclopentyl, cyclohexyl, piperidinyl, piperazinyl, morpholinyl, phenyl, pyrrolyl, pyrazolyl, imidazolyl, thienyl, furyl, oxazolyl, isoxazolyl, thiazolyl, isothiazolyl and pyridyl; where the radicals may be unsubstituted or mono- or polysubstituted (e.g., two, three, four or five times) with R⁸ radicals; and the C₃₋₇-cycloalkyl, phenyl and 5- to 7-membered heteroaryl radicals may be bound to the cyclodextrin compound via a C₁₋₄-alkylene bridge, preferably —CH₂—, —CH₂—CH₂—, —CH₂—CH₂—CH₂—, or —CH₂—CH₂—CH₂—CH₂—.

R⁸ is preferably selected from the group consisting of —C(═O)H, —C(═O)W, —C(═O)OH, —C(═O)OW, —C(═O)NH₂, —C(═O)NHW, —C(═O)NW₂, —C(═O)NHC(═O)W, —C(═O)NWC(═O)W, —OH, —OW, —OV, —OC(═O)H, —OC(═O)W, —OC(═O)OH, —OC(═O)OW, —OC(═O)NH₂, —OC(═O)NHW, —OC(═O)NW₂, —O—N═O, —OS(═O)H, —OS(═O)W, —OS(═O)OH, —OS(═O)OW, —OS(═O)₂H, —OS(═O)₂W, —OS(═O)₂OH, —OS(═O)₂OW, —OS(═O)NH₂, —OS(═O)NHW, —OS(═O)NW₂, —OS(═O)₂NH₂, —OS(═O)₂—NHW, —OS(═O)₂NW₂, —OP(═O)(W)₂, —OP(═O)(OH)(W), —OP(═O)(OW)(W), —OP(═O) (OH)₂, —OP(═O)(OW)(OH), —OP(═O)(OW)₂, —O—P(═NH)(W)₂, —OP(═NW)(W)₂, —OP(═O)(NH₂)₂, —OP(═O)(NW)(NH₂), —OP(═S)(W)₂, —OP(═S)(OH)(W), —OP(═S) (OW)(W), —OP(═S)(OH)₂, —OP(═S)(OW)(OH), —OP(═S)(OW)₂, —SH, —SW, —SV, —S(═O)H, —S(═O)W, —S(═O)OH, —S(═O)OW, —S(═O)₂H, —S(═O)₂W, —S(═O)₂OH, —S(═O)₂OW, —S(═O)NH₂, —S(═O)NHW, —S(═O)NW₂, —S(═O)₂NH₂, —S(═O)₂NHW, —S(═O)₂NW₂, —NH₂, —NHW, —NHV, —NW₂, —NV₂, —N(W)₃⁺A⁻, —NHC(═O)H, —NHC(═O)W, N[C(═O)W]₂, —NWC(═O)W, —NHC(═O)NH₂, —NHC(═O)NHW, —NWC(═O)NHW, —NWC(═O)NW₂, —NWC(═O)NW₂, —NHC(═O)OH, —NHC(═O)OW, —NWC(═O)OW, —NHS(═O)H, —NHS(═O)W, —NWS(═O)H, —NWS(═O)W, —NHS(═O)OH, —NHS(═O)OW, —NWS(═O)OH, —NWS(═O)OW, —NHS(═O)₂H, —NHS(═O)₂W, —NWS(═O)₂H, —NWS(═O)₂W, —NHS(═O)₂OH, —NHS(═O)₂OW, —NWS(═O)₂OH, —NWS(═O)₂OW, —NHS(═O)NH₂, —NWS(═O)NHW, —NHS(═O)NW₂, —NWS(═O)NH₂, —NWS(═O)NW₂, —NHS(═O)₂NH₂, —NHS(═O)₂NHW, —NHS(═O)₂NW₂, —NWS(═O)₂NH₂, —NWS(═O)₂NHW, —NWS(═O)₂NW₂, ═N—OH, ═N—NH₂, —N═N—W, —PH₂, —PHW, —PW₂, —P(═O)W₂, —P(═O)(OH)(W), —P(═O)(OW)(W), —P(═O)(OH)₂, —P(═O)(OW)(OH), —P(═O)(OW)₂, —P(═NH)(W)₂, —P(═NW)(W)₂, —P(═O)(NH₂)₂, —P(═O)(NW₂)₂, —P(═S)(W)₂, —P(═S) (OH)(W), —P(═S)(OW)W, —P(═S)(OH)₂, —P(═S)(OW)(OH), —P(═S)(OW)₂, ═NH, ═NW, ═O, ═S, pyrrolidinyl, pyrazolinyl, pyrazolidinyl, imidazolinyl, imidazolidinyl, dioxolanyl, piperidinyl, piperazinyl, 4-W-piperazinyl, dioxanyl, morpholinyl, quinuclidinyl, pyrazolinonyl, indolinyl, isoindolinyl, quinolizinyl, benzodioxolanyl, pyrrolyl, pyrazolyl, imidazolyl, thienyl, oxazolyl, isoxazolyl, thiazolyl, isothiazolyl, furazanyl, oxadiazolyl, pyridyl, pyridazinyl, pyrazinyl, pyrimidinyl, indolyl, isoindolyl, indolizinyl, indazolyl, benzotriazolyl, quinolinyl, isoquinolinyl, naphthyridinyl, quinazolinyl, phthalazinyl, quinoxalinyl, purinyl, pteridinyl, benzothienyl, benzothiazolyl and benzothiadiazolyl; where the W radical preferably stands for methyl, ethyl, N-propyl, isopropyl, isobutyl, N-butyl, sec-butyl, tert-butyl, cyclopropyl, cyclobutyl, cyclopentyl, cyclohexyl, allyl, vinyl or ethynyl; the V radical stands for a hybrid radical; and A⁻ stands for an organic or inorganic anion, which is preferably selected from the group consisting of Cl⁻, Br⁻, I⁻, dihydrogen phosphate, hydrogen phosphate, phosphate, bicarbonate, carbonate, acetate, lactate and citrate or for other physiologically tolerable anions.

In another preferred embodiment, R⁸ is selected from the group consisting of —C(═O)H, —C(═O)W, —C(═O)OH, —C(═O)OW, —C(═O)NH₂, —C(═O)NHW, —C(═O)NW₂, —C(═O)NHC(═O)W, —C(═O)NWC(═O)W, —OH, —OW, —OV, —OC(═O)H, —OC(═O)W, —OC(═O)OH, —OC(═O)OW, —OC(═O)NH₂, —OC(═O)NHW, —OC(═O)NW₂, —OS(═O)H, —OS(═O)W, —OS(═O)OH, —OS(═O)OW, —OS(═O)₂H, —OS(═O)₂W, —OS(═O)₂OH, —OS(═O)₂OW, —OS(═O)NH₂, —OS(═O)NHW, —OS(═O)NW₂, —OS(═O)₂NH₂, —OS(═O)₂NHW, —OS(═O)₂NW₂, —OP(═O)(OH)(W), —OP(═O)(OH)₂, —OP(═O)(OW)(OH), —OP(═O)(OW)₂, —SH, —SV, —S(═O)H, —S(═O)W, —S(═O)OH, —S(═O)OW, —S(═O)₂H, —S(═O)₂W, —S(═O)₂OH, —S(═O)₂OW, —S(═O)NH₂, —S(═O)NHW, —S(═O)NW₂, S(═O)₂NH₂, —S(═O)₂NHW, —S(═O)₂NW₂, —NH₂, —NHW, —NHV, —NW₂, —NV₂, —N(W)₃⁺A⁻, —NHC(═O)H, —NHC(═O)W, —N[C(═O)W]₂, —NWC(═O)W, —NHC(═O)NH₂, —NHC(═O)NHW, —NWC(═O)NW₂, —NWC(═O)NHW, —NWC(═O)NW₂, —NHC(═O)OH, —NHC(═O)OW, —NWC(═O)OW, —NHS(═O)H, —NWS(═O)H, —NHS(═O)OH, —NWS(═O)OH, —NHS(═O)₂OH, —NWS(═O)₂OH, ═N—OH, ═N—NH₂, —PH₂, —P(═O)W₂, —P(═O)(OH)(W), —P(═O)(OH)₂, —P(═O)(OW)(OH), —P(═O)(OW)₂, ═NH, ═NW, ═O, ═S, pyrrolidinyl, pyrazolinyl, pyrazolidinyl, imidazolinyl, imidazolidinyl, piperidinyl, piperazinyl, 4-W-piperazinyl, morpholinyl, quinuclidinyl, pyrazolinonyl, imidazolyl; where the W radical preferably stands for methyl, ethyl, N-propyl, isopropyl, cyclopropyl, allyl, vinyl or ethynyl; the V radical stands for a hybrid radical; and A⁻ stands for an organic or inorganic anion, which is preferably selected from the group consisting of Cl⁻, Br⁻, I⁻, dihydrogen phosphate, hydrogen phosphate, phosphate, bicarbonate, carbonate, acetate, lactate and citrate or for other physiologically tolerable anions.

In another preferred embodiment, R⁸ stands for an ionic, ionizable hydroxyl or polyol radical. In this context, R⁸ is preferably selected from the group consisting of —C(═O)—OH, —OH, —OV, —OC(═O)OH, —OC(═O)NH₂, —OS(═O)OH, —OS(═O)₂OH, —OP(═O) (OH)(W), —OP(═O)(OH)₂, —OP(═O)(OW)(OH), —OP(═O)(OW)₂, —SH, —SV, —S(═O)OH, —S(═O)₂OH, —NH₂, —NHW, —NHV, —NW₂, —NV₂, —N(W)₃⁺A⁻, —NHC(═O)OH, —NWC(═O)—OH, —NHS(═O)OH, —NWS(═O)OH, —NHS(═O)₂OH, —NWS(═O)₂OH, ═N—OH, ═N—NH₂, —P(═O)(OH)(W), —P(═O)(OH)₂, —P(═O)(OW)(OH), —P(═O)(OW)₂, ═NH, ═NW, piperidinyl, morpholinyl and imidazolyl; where the W radical is selected from the group consisting of methyl, ethyl, N-propyl, isopropyl, cyclopropyl, allyl, vinyl and ethynyl; the V radical stands for a hybrid radical; and A stands for an organic or inorganic anion, which is preferably selected from the group consisting of Cl⁻, Br⁻, I⁻, dihydrogen phosphate, hydrogen phosphate, phosphate, bicarbonate, carbonate, acetate, lactate and citrate or for other physiologically tolerable anions.

In another preferred embodiment, R⁸ stands for an ionic, ionizable hydroxyl or polyol radical. In this context, R⁸ is preferably selected from the group consisting of —C(═O)—OH, —OH, —OV, —OS(═O)₂OH, —OP(═O)(OH)₂, —SH, —SV, —S(═O)₂OH, —NH₂, —NHW, —NHV, —NW₂, —NV₂, —N(W)₃⁺A⁻, —NHC(═O)OH, —NWC(═O)OH, —P(═O)(OH)(W), —P(═O)(OH)₂, —P(═O)(OW)(OH); where the W radical is selected from the group consisting of methyl, ethyl, N-propyl, isopropyl, cyclopropyl, allyl, vinyl and ethynyl; the V radical stands for a hybrid radical; and A⁻ stands for an organic or inorganic anion, which is preferably selected from the group consisting of Cl⁻, Br⁻, I⁻, dihydrogen phosphate, hydrogen phosphate, phosphate, bicarbonate, carbonate, acetate, lactate and citrate or for other physiologically tolerable anions.

For the purposes of this invention, an “ionic radical” is understood to refer to substituents having a permanent cationic or anionic, preferably cationic charge (e.g., quaternary ammonium; —N(CH₃)₃⁺, where the symbol “—” stands for covalent single bond to a C atom).

In the sense of this invention, the term “ionizable radical” includes substituents which have an anionic charge by releasing a proton (e.g., carboxyl→carboxylate) or have a cationic charge by receiving a proton (e.g., primary, secondary, or tertiary amine primary, secondary, or tertiary ammonium).

For the purposes of this invention, the term “polyol” comprises C₁₋₆-alkyl substituents having at least two hydroxyl radicals (e.g., two, three, four or five) on the same or different carbon atoms of the alkyl (e.g., —CH₂—CH(OH)—CH₂—CH₂—OH).

In another preferred embodiment, the R⁶ and R⁷ radicals, independently of one another, are selected from the group consisting of methyl, ethyl, isopropyl, N-propyl, isobutyl, N-butyl, sec-butyl, tert-butyl, ethenyl, ethynyl, prop-1-enyl, cyclopropyl, cyclobutyl, cyclopentyl, cyclohexyl, phenyl, benzyl, phenethyl, —CH₂—C(═O)—OH, —(CH₂)₂—C(═O)—OH, —(CH₂)₃—C(═O)—OH, —(CH₂)₄—C(═O)—OH, —(CH₂)₅—C(═O)—OH, —(CH₂)₆—C(═O)—OH, —CH₂—CH(OH)—C(═O)OH, —CH₂—CH(OH)—C(═O)O—CH₃, —CH₂—CH(OH)—C(═O)O—CH₂—CH₃, —CH(CH₂OH)—C(═O)OH, —CH(CH₂OH)—C(═O)O—CH₃, —CH(CH₂—OH)—C(═O)O—CH₂—CH₃, —CH₂—CH(OH)—CH₂—C(═O)—OH, —CH₂—CH(OH)—CH₂—C(═O)—O—CH₃, —CH₂—CH(OH)—CH₂—C(═O)—OCH₂—CH₃, —CH(CH₂—OH)—CH₂—C(═O)—O—CH₃, —CH(CH₂—OH)—CH₂—C(═O)—O—CH₂—CH₃, —CH₂—OH, —CH(OH)—CH₃, —(CH₂)₂—OH, —CH(OH)—CH₂—CH₃, —CH₂—CH(OH)—CH₃, —(CH₂)₃—OH, —CH(CH₃)—CH₂—OH, —CH(OH)—(CH₂)₂—CH₃, —CH₂—CH(OH)—CH₂—CH₃, —(CH₂)₂—CH(OH)—CH₃, —(CH₂)₄—OH, —(CH₂)₅—OH, —(CH₂)₆—OH, —CH(OH)—CH₂(OH), —CH(OH)—CH(OH)—CH₃, —CH(OH)—CH₂—CH₂(OH), —CH₂—CH(OH)—CH₂(OH), —CH(OH)—CH(OH)—CH₂—CH₃, —CH(OH)—CH₂CH(OH)—CH₃, —CH(OH)—(CH₂)₂—CH₂(OH), —CH₂—CH(OH)—CH(OH)—CH₃, —CH₂—CH(OH)—CH₂—CH₂(OH), (CH₂)₂—CH(OH)—CH₂(OH), —CH(OH)—CH(OH)—CH₂(OH), —CH(OH)—CH(OH)—CH(OH)—CH₃, —CH(OH)—CH(OH)—CH₂—CH₂(OH), —CH(OH)—CH₂—CH(OH)—CH₂(OH), —CH₂—CH(OH)—CH(OH)—CH₂(OH), —CH(OH)—CH(OH)—CH(OH)—CH₂(OH), —CH₂—O—S(═O)₂OH, —(CH₂)₂—O—S(═O)₂OH, —(CH₂)₃—O—S(═O)₂OH, —(CH₂)₄—O—S(═O)₂OH, —(CH₂)₅—O—S(═O)₂OH, —(CH₂)₆—O—S(═O)₂OH, —CH₂—O—P(═O)(OH)₂, —(CH₂)₂—O—P(═O)(OH)₂, —(CH₂)₃—O—P(═O)(OH)₂, —(CH₂)₄—O—P(═O)(OH)₂, —(CH₂)₅—O—P(═O)(OH)₂, —(CH₂)₆—O—P(═O)(OH)₂, —CH₂—SH, —CH(SH)—CH₃, —(CH₂)₂—SH, —CH(SH)—CH₂—CH₃, —CH₂—CH(SH)—CH₃, —(CH₂)₃—SH, —CH(SH)—(CH₂)₂—CH₃, —CH₂—CH(SH)—CH₂—CH₃, —(CH₂)₂—CH(SH)—CH₃, —(CH₂)₄—SH, —(CH₂)₅—SH, —(CH₂)₆—SH, —CH(SH)—CH₂(SH), —CH(SH)—CH(SH)—CH₃, —CH(SH)—CH₂—CH₂(SH), —CH₂—CH(SH)—CH₂(SH), —CH(SH)—CH(SH)—CH₂—CH₃, —CH(SH)—CH₂CH(SH)—CH₃, —CH(SH)—(CH₂)₂—CH₂(SH), —CH₂—CH(SH)—CH(SH)—CH₃, —CH₂—CH(SH)—CH₂—CH₂(SH), —(CH₂)₂—CH(SH)—CH₂(SH), —CH(SH)—CH(SH)—CH₂(SH), —CH(SH)—CH(SH)—C(SH)—CH₃, —CH(SH)—CH(SH)—CH₂—CH₂(SH), —CH(SH)—CH₂—CH(SH)—CH₂(SH), —CH₂—CH(SH)—CH(SH)—CH₂(SH), —CH(SH)—CH(SH)—CH(SH)—CH₂(SH), —CH₂—S(═O)₂OH, —(CH₂)₂—S(═O)₂OH, —(CH₂)₃—S(═O)₂OH, —(CH₂)₄—S(═O)₂OH, —(CH₂)₅—S(═O)₂OH, —(CH₂)₆—S(═O)₂OH, —CH₂—NH₂, —(CH₂)₂—NH₂, —(CH₂)₃—NH₂, —(CH₂)₄—NH₂, —(CH₂)₅—NH₂, —(CH₂)₆—NH₂, —CH₂—NH(CH₃), —(CH₂)₂—NH(CH₃), —(CH₂)₃—NH(CH₃), —(CH₂)₄—NH(CH₃), —(CH₂)₅—NH(CH₃), —(CH₂)₆—NH(CH₃), —CH₂—NH(CH₂CH₃), —(CH₂)₂—NH(CH₂CH₃), —(CH₂)₃—NH(CH₂CH₃), —(CH₂)₄—NH(CH₂CH₃), —(CH₂)₅—NH(CH₂CH₃), —(CH₂)₆—NH(CH₂CH₃), —CH₂—N(CH₃)₂, —(CH₂)₂—N(CH₃)₂, —(CH₂)₃—N(CH₃)₂, —(CH₂)₄—N(CH₃)₂, —(CH₂)₅—N(CH₃)₂, —(CH₂)₆—N(CH₃)₂, —CH₂—N(CH₂CH₃)₂, —(CH₂)₂—N(CH₂CH₃)₂, —(CH₂)₃—N(CH₂CH₃)₂, —(CH₂)₄—N(CH₂CH₃)₂, —(CH₂)₅—N(CH₂CH₃)₂, —(CH₂)₆—N(CH₂CH₃)₂, —CH₂—N(CH₃)₃⁺A⁻, —(CH₂)₂—N(CH₃)₃⁺A⁻, —(CH₂)₃—N(CH₃)₃⁺A⁻, —(CH₂)₄—N(CH₃)₃⁺A⁻, —(CH₂)₅—N(CH₃)₃⁺A⁻, —(CH₂)₆—N(CH₃)₃⁺A⁻, —CH₂—N(CH₂CH₃)₃⁺A⁻, —(CH₂)₂—N(CH₂CH₃)₃⁺A⁻, —(CH₂)₃—N(CH₂CH₃)₃⁺A⁻, —(CH₂)₄—N(CH₂CH₃)₃⁺A⁻, —(CH₂)₅—N(CH₂CH₃)₃⁺A⁻, —(CH₂)₆—N(CH₂CH₃)₃⁺A⁻, —CH₂—P(═O)(OH)₂, —(CH₂)₂—P(═O)(OH)₂, —(CH₂)₃—P(═O)(OH)₂, —(CH₂)₄—P(═O)(OH)₂, —(CH₂)₅—P(═O)(OH)₂, —(CH₂)₆—P(═O)(OH)₂, —CH₂-pyrrolyl, —(CH₂)₂-pyrrolyl, —(CH₂)₃-pyrrolyl, —(CH₂)₄-pyrrolyl, —CH₂-pyrazolyl, —(CH₂)₂-pyrazolyl, —(CH₂)₃-pyrazolyl, —(CH₂)₄-pyrazolyl, —CH₂-imidazolyl, —(CH₂)₂-imidazolyl, —(CH₂)₃-imidazolyl, —(CH₂)₄-imidazolyl, —CH₂-oxazolyl, —(CH₂)₂-oxazolyl, —(CH₂)₃-oxazolyl, —(CH₂)₄-oxazolyl, —CH₂-isoxazolyl, —(CH₂)₂-isoxazolyl, —(CH₂)₃-isoxazolyl, —(CH₂)₄-isoxazolyl, —CH₂-isothiazolyl, —(CH₂)₂-isothiazolyl, —(CH₂)₃-isothiazolyl, —(CH₂)₄-isothiazolyl, —CH₂-pyridyl, —(CH₂)₂-pyridyl, —(CH₂)₃-pyridyl and —(CH₂)₄-pyridyl; where A⁻ stands for an organic or inorganic anion, which is preferably selected from the group consisting of Cl⁻, Br⁻, I⁻, dihydrogen phosphate, hydrogen phosphate, phosphate, bicarbonate, carbonate, acetate, lactate and citrate or for other physiologically tolerable anions; where the radicals may optionally be further substituted with hybrid radicals.

In another preferred embodiment, each individual K radical stands for either hydrogen or for a radical selected from the group consisting of glucose, galactose, maltose, maltotriose, —C(═O)—C₁₋₆-alkyl, —C(═O)—C₃₋₇-cycloalkyl, —C(═O)-phenyl, —C(═O)-(5- to 7-membered heteroaryl), —C₁₋₆-alkyl, —C₃₋₇-cycloalkyl, -phenyl and -(5- to 7-membered heteroaryl); where the C₃₋₇-cycloalkyl, phenyl and 5- to 7-membered heteroaryl radicals may be bound to the cyclodextrin monomers C₁₋₄-alkylene bridge, preferably —CH₂—, —CH₂—CH₂—, —CH₂—CH₂—CH₂—, or —CH₂—CH₂—CH₂—CH₂—; and C₁₋₆-alkyl, alkyl, phenyl and 5- to 7-membered heteroaryl may be unsubstituted or mono- or polysubstituted (e.g., two, three, four or five times) with the same or different R⁸ radicals, where R⁸ is selected from the group consisting of —C(═O)OH, —OH, —OV, —OC(═O)OH, —OC(═O)NH₂, —OS(═O)OH, —OS(═O)₂OH, —OP(═O)(OH)(W), —OP(═O)(OH)₂, —OP(═O)(OW)(OH), —OP(═O)(OW)₂, —SH, —SV, —S(═O)OH, —S(═O)₂OH, —NH₂, —NHW, —NHV, —NW₂, —NV₂, —N(W)₃⁺A⁻, —NHC(═O)OH, —NWC(═O)OH, —NHS(═O)OH, —NWS(═O)OH, —NHS(═O)₂OH, —NWS(═O)₂OH, ═N—OH, ═N—NH₂, —P(═O)(OH)(W), —P(═O)(OH)₂, —P(═O)(OW)(OH), —P(═O)(OW)₂, ═NH, ═NW, pyrrolidinyl, pyrazolinyl, pyrazolidinyl, imidazolinyl, imidazolidinyl, piperidinyl, piperazinyl, 4-W-piperazinyl, morpholinyl, quinuclidinyl, pyrazolinonyl and imidazolyl; where the W radical is selected from the group consisting of methyl, ethyl, N-propyl, isopropyl, cyclopropyl, allyl, vinyl and ethynyl; the V radical stands for a hybrid radical; and A⁻ stands for an organic or inorganic anion, which is preferably selected from the group consisting of Cl⁻, Br⁻, I⁻, dihydrogen phosphate, hydrogen phosphate, phosphate, bicarbonate, carbonate, acetate, lactate and citrate or for other physiologically tolerable anions.

In another preferred embodiment, each individual K radical stands for either hydrogen or for a radical selected from the group consisting of glucose, galactose, maltose, maltotriose, —C(═O)-methyl, —C(═O)-ethyl, —C(═O)-isopropyl, —C(═O)—N-propyl, —C(═O)-isobutyl, —C(═O)—N-butyl, —C(═O)-sec-butyl, —C(═O)-tert-butyl, —C(═O)—N-pentyl, —C(═O)-isopentyl, —C(═O)-neopentyl, —C(═O)-isohexyl, —C(═O)—N-hexyl, —C(═O)-neohexyl, —C(═O)-(3-methylpentyl), —C(═O)-(2,3-dimethylbutyl), —C(═O)-ethenyl, —C(═O)ethynyl, —C(═O)-prop-1-enyl, —C(═O)-cyclopropyl, —C(═O)-cyclobutyl, —C(═O)-cyclopentyl, —C(═O)-cyclohexyl, —C(═O)-piperidinyl, —C(═O)-piperazinyl, —C(═O)-morpholinyl, —C(═O)-phenyl, —C(═O)-imidazolyl, —C(═O)-thienyl, —C(═O)-furyl, —C(═O)-oxazolyl, —C(═O)-isoxazolyl, —C(═O)-thiazolyl, —C(═O)-isothiazolyl, —C(═O)-pyridyl, methyl, ethyl, isopropyl, N-propyl, isobutyl, N-butyl, sec-butyl, tert-butyl, N-pentyl, isopentyl, neopentyl, isohexyl, N-hexyl, neohexyl, 3-methyl-pentyl, 2,3-dimethylbutyl, ethenyl, ethynyl, prop-1-enyl, cyclopropyl, cyclobutyl, cyclopentyl, cyclohexyl, piperidinyl, piperazinyl, morpholinyl, phenyl, pyrrolyl, pyrazolyl, imidazolyl, thienyl, furyl, oxazolyl, isoxazolyl, thiazolyl, isothiazolyl and pyridyl; where the C₃₋₇-cycloalkyl, phenyl and 5- to 7-membered heteroaryl radicals may be bound to the corresponding cyclodextrin monomers via a C₁₋₄-alkylene bridge, preferably —CH₂—, —CH₂—CH₂—, —CH₂—CH₂—CH₂—, or —CH₂—CH₂—CH₂—CH₂—; and the C₁₋₆-alkyl, C₃₋₇-cycloalkyl, phenyl and 5- to 7-membered heteroaryl radicals may be unsubstituted or mono- or polysubstituted (e.g., two, three, four or five times) with R⁸ radicals where R⁸ is preferably selected from the group consisting of —C(═O)OH, —OH, —OV, —OS(═O)₂OH, —OP(═O)(OH)₂, —SH, —SV, —S(═O)₂OH, —NH₂, —NHW, —NHV, —NW₂, —NV₂, —N(W)₃⁺A⁻, —NHC(═O)OH, —NWC (═O)OH, —P(═O)(OH)(W), —P(═O)(OH)₂, —P(═O)(OW)(OH), piperidinyl, piperazinyl, morpholinyl and imidazolyl; where the W radical is selected from the group consisting of methyl, ethyl, N-propyl, isopropyl, cyclopropyl, allyl, vinyl and ethynyl; the V radical stands for a hybrid radical; and A⁻ stands for an organic or inorganic anion, which is preferably selected from the group consisting of Cl⁻, Br⁻, I⁻, dihydrogen phosphate, hydrogen phosphate, phosphate, bicarbonate, carbonate, acetate, lactate and citrate or for other physiologically tolerable anions.

In another preferred embodiment, each individual K radical stands for either hydrogen or for a radical selected from the group consisting of glucose, galactose, maltose, maltotriose, —C(═O)-methyl, —C(═O)-ethyl, —C(═O)-isopropyl, —C(═O)—N-propyl, —C(═O)-isobutyl, —C(═O)—N-butyl, —C(═O)-sec-butyl, —C(═O)-tert-butyl, —C(═O)-ethenyl, —C(═O)-ethynyl, —C(═O)-prop-1-enyl, —C(═O)-cyclopropyl, —C(═O)-cyclobutyl, —C(═O)cyclopentyl, —C(═O)-cyclohexyl, —C(═O)-phenyl, —C(═O)-benzyl, —C(═O)-phenethyl, —C(═O)—CH₂—C(═O)—OH, —C(═O)—(CH₂)₂—C(═O)—OH, —C(═O)—(CH₂)₃—C(═O)—OH, —C(═O)—(CH₂)₄—C(═O)—OH, —C(═O)—(CH₂)₅—C(═O)—OH, —C(═O)—(CH₂)₆—C(═O)—OH, —C(═O)—CH₂—OH, —C(═O)—CH(OH)—CH₃, —C(═O)—(CH₂)₂—OH, —C(═O)—CH(OH)—CH₂—CH₃, —C(═O)—CH₂—CH(OH)—CH₃, —C(═O)—(CH₂)₃—OH, —C(═O)—CH(OH)—(CH₂)₂—CH₃, —C(═O)—CH₂—CH(OH)—CH₂—CH₃, —C(═O)—(CH₂)₂—CH(OH)—CH₃, —C(═O)—(CH₂)₄—OH, —C(═O)—(CH₂)₅—OH, —C(═O)—(CH₂)₆—OH, —C(═O)—CH(OH)—CH₂(OH), —C(═O)—CH(OH)—CH(OH)—CH₃, —C(═O)—CH(OH)—CH₂—CH₂(OH), —C(═O)—CH₂—CH(OH)—CH₂(OH), —C(═O)—CH(OH)—CH(OH)—CH₂—CH₃, —C(═O)—CH(OH)—CH₂—CH(OH)—CH₃, —C(═O)—CH(OH)—(CH₂)₂—CH₂(OH), —C(═O)—CH₂—CH(OH)—CH(OH)—CH₃, —C(═O)—CH₂—CH(OH)—CH₂—CH₂(OH), —C(═O)—(CH₂)₂—CH(OH)—CH₂(OH), —C(═O)—CH(OH)—CH(OH)—CH₂(OH), —C(═O)—CH(OH)—CH(OH)—CH(OH)—CH₃, —C(═O)—CH(OH)—CH(OH)—CH₂—CH₂(OH), —C(═O)—CH(OH)—CH₂—CH(OH)—CH₂(OH), —C(═O)—CH₂—CH(OH)—CH(OH)—CH₂(OH), —C(═O)—CH(OH)—CH(OH)—CH(OH)—CH₂(OH), —C(═O)—CH₂—NH₂, —C(═O)—(CH₂)₂—NH₂, —C(═O)—(CH₂)₃—NH₂, —C(═O)—(CH₂)₄—NH₂, —C(═O)—(CH₂)₅—NH₂, —C(═O)—(CH₂)₆—NH₂, —C(═O)—CH₂—NH(CH₃), —C(═O)—(CH₂)₂—NH(CH₃), —C(═O)—(CH₂)₃—NH(CH₃), —C(═O)—(CH₂)₄—NH(CH₃), —C(═O)—(CH₂)₅—NH(CH₃), —C(═O)—(CH₂)₆—NH(CH₃), —C(═O)—CH₂—NH(CH₂CH₃), —C(═O)—(CH₂)₂—NH(CH₂CH₃), —C(═O)—(CH₂)₃—NH(CH₂—CH₃), —C(═O)—(CH₂)₄—NH(CH₂CH₃), —C(═O)—(CH₂)₅—NH(CH₂CH₃), —C(═O)—(CH₂)₆—NH(CH₂CH₃), —C(═O)—CH₂—N(CH₃)₂, —C(═O)—(CH₂)₂—N(CH₃)₂, —C(═O)—(CH₂)₃—N(CH₃)₂, —C(═O)—(CH₂)₄—N(CH₃)₂, —C(═O)—(CH₂)₅—N(CH₃)₂, —C(═O)—(CH₂)₆—N(CH₃)₂, —C(═O)—CH₂—N(CH₂CH₃)₂, —C(═O)—(CH₂)₂—N(CH₂—CH₃)₂, —C(═O)—(CH₂)₃—N(CH₂CH₃)₂, —C(═O)—(CH₂)₄—N(CH₂CH₃)₂, —C(═O)—(CH₂)₅—N(CH₂CH₃)₂, —C(═O)—(CH₂)₆—N(CH₂CH₃)₂, methyl, ethyl, isopropyl, N-propyl, isobutyl, N-butyl, sec-butyl, tert-butyl, ethenyl, ethynyl, prop-1-enyl, cyclopropyl, cyclobutyl, cyclopentyl, cyclohexyl, phenyl, benzyl, phenethyl, —CH₂—C(═O)—OH, —(CH₂)₂—C(═O)—OH, —(CH₂)₃—C(═O)—OH, —(CH₂)₄—C(═O)—OH, —(CH₂)₅—C(═O)—OH, —(CH₂)₆—C(═O)—OH, —CH₂—CH(OH)—C(═O)OH, —CH₂—CH(OH)—C(═O)O—CH₃, —CH₂—CH(OH)—C(═O)O—CH₂—CH₃, —CH(CH₂OH)—C(═O)OH, —CH (CH(OH)—C(═O)O—CH₃, —CH(CH₂—OH)—C(═O)O—CH₂—CH₃, —CH₂—CH(OH)—CH₂—C(═O)—OH, —CH₂—CH(OH)—CH₂—C(═O)—O—CH₃, —CH₂—CH(OH)—CH₂—C(═O)—OCH₂—CH₃, —CH(CH₂—OH)—CH₂—C(═O)—O—CH₃, —CH(CH₂—OH)—CH₂—C(═O)—O—CH₂—CH₃, —CH₂—OH, —CH(OH)—CH₃, —(CH₂)₂—OH, —CH(OH)—CH₂—CH₃, —CH₂—CH(OH)—CH₃, —(CH₂)₃—OH, —CH(CH₃)—CH₂—OH, —CH(OH)—(CH₂)₂—CH₃, —CH₂—CH(OH)—CH₂—CH₃, —(CH₂)₂—CH(OH)—CH₃, —(CH₂)₄—OH, —(CH₂)₅—OH, —(CH₂)₆—OH, —CH(OH)—CH₂(OH), —CH(OH)—CH(OH)—CH₃, —CH(OH)—CH₂—CH₂(OH), —CH₂—CH(OH)—CH₂(OH), —CH(OH)—CH(OH)—CH₂—CH₃, —CH(OH)—CH₂—CH(OH)—CH₃, —CH(OH)—(CH₂)₂—CH₂(OH), —CH₂—CH(OH)—CH(OH)—CH₃, —CH₂—CH(OH)—CH₂—CH₂(OH), —(CH₂)₂—CH(OH)—CH₂(OH), —CH(OH)—CH(OH)—CH₂(OH), —CH(OH)—CH(OH)—CH(OH)—CH₃, —CH(OH)—CH(OH)—CH₂—CH₂(OH), —CH(OH)—CH₂—CH(OH)—CH₂(OH), —CH₂—CH(OH)—CH(OH)—CH₂(OH), —CH(OH)—CH(OH)—CH(OH)—CH₂(OH), —CH₂—O—S(═O)₂OH, —(CH₂)₂—O—S(═O)₂OH, —(CH₂)₃—O—S(═O)₂OH, —(CH₂)₄—O—S(═O)₂OH, —(CH₂)₅—O—S(═O)₂OH, —(CH₂)₆—O—S(═O)₂OH, —CH₂—O—P(═O)(OH)₂, —(CH₂)₂—O—P(═O)(OH)₂, —(CH₂)₃—O—P(═O)(OH)₂, —(CH₂)₄—O—P(═O)(OH)₂, —(CH₂)₅—O—P(═O)(OH)₂, —(CH₂)₆—O—P(═O)(OH)₂, —CH₂—SH, —CH(SH)—CH₃, —(CH₂)₂—SH, —CH(SH)—CH₂—CH₃, —CH₂—CH(SH)—CH₃, —(CH₂)₃—SH, —CH(SH)—(CH₂)₂—CH₃, —CH₂—CH(SH)—CH₂—CH₃, —(CH₂)₂—CH(SH)—CH₃, —(CH₂)₄—SH, —(CH₂)₅—SH, —(CH₂)₆—SH, —CH₂—S(═O)₂OH, —(CH₂)₂—S(═O)₂OH, —(CH₂)₃—S(═O)₂OH, —(CH₂)₄—S(═O)₂OH, —(CH₂)₅—S(═O)₂OH, —(CH₂)₆—S(═O)₂OH, —CH₂—NH₂, —(CH₂)₂—NH₂, —(CH₂)₃—NH₂, —(CH₂)₄—NH₂, —(CH₂)₅—NH₂, —(CH₂)₆—NH₂, —CH₂—NH(CH₃), —(CH₂)₂—NH(CH₃), —(CH₂)₃—NH(CH₃), —(CH₂)₄—NH(CH₃), —(CH₂)₅—NH(CH₃), —(CH₂)₆—NH(CH₃), —CH₂—NH(CH₂CH₃), —(CH₂)₂—NH(CH₂CH₃), —(CH₂)₃—NH(CH₂CH₃), —(CH₂)₄—NH(CH₂CH₃), —(CH₂)₅—NH(CH₂CH₃), —(CH₂)₆—NH(CH₂CH₃), —CH₂—N(CH₃)₂, —(CH₂)₂—N(CH₃)₂, —(CH₂)₃—N(CH₃)₂, —(CH₂)₄—N(CH₃)₂, —(CH₂)₅—N(CH₃)₂, —(CH₂)₆—N(CH₃)₂, —CH₂—N(CH₂CH₃)₂, —(CH₂)₂—N(CH₂CH₃)₂, —(CH₂)₃—N(CH₂CH₃)₂, —(CH₂)₄—N(CH₂CH₃)₂, —(CH₂)₅—N(CH₂CH₃)₂, —(CH₂)₆—N(CH₂CH₃)₂, —CH₂—N(CH₃)₃⁺A⁻, —(CH₂)₂—N(CH₃)₃⁺A⁻, —(CH₂)₃—N(CH₃)₃⁺A⁻, —(CH₂)₄—N(CH₃)₃⁺A⁻, —(CH₂)₅—N(CH₃)₃⁺A⁻, —(CH₂)₆—N(CH₃)₃⁺A⁻, —CH₂—N(CH₂CH₃)₃⁺A⁻, —(CH₂)₂—N(CH₂CH₃)₃⁺A⁻, —(CH₂)₃—N(CH₂CH₃)₃⁺A⁻, —(CH₂)₄—N(CH₂CH₃)₃⁺A⁻, —(CH₂)₅—N(CH₂CH₃)₃⁺A⁻, —(CH₂)₆—N(CH₂CH₃)₃⁺A⁻, —CH₂—P(═O)(OH)₂, —(CH₂)₂—P(═O)(OH)₂, —(CH₂)₃—P(═O)(OH)₂, —(CH₂)₄—P(═O)(OH)₂, —(CH₂)₅—P(═O)(OH)₂, —(CH₂)₆—P(═O)(OH)₂; where A⁻ stands for an organic or inorganic anion, which is preferably selected from the group consisting of Cl⁻, Br⁻, I⁻, dihydrogen phosphate, hydrogen phosphate, phosphate, bicarbonate, carbonate, acetate, lactate and citrate or for other physiologically tolerable anions; where the radicals may optionally be further substituted with hybrid radicals.

The term “crosslinking agent” in the sense of this invention stands for compounds containing two or more reactive groups (e.g., 3, 4, 5 or 6), which may be the same or different, such as —Cl, —Br, —I, -tosyl, -mesyl, —N═C═O, ═O, —C(═O)H, —C(═O)Cl or epoxy and are thus suitable for crosslinking the cyclodextrin compounds. The reactive groups of the crosslinking agents are also referred to below as leaving groups.

The crosslinking agents are preferably bifunctional or polyfunctional crosslinking agents, more preferably homobifunctional, homopolyfunctional, heterobifunctional or heteropolyfunctional crosslinking agents and in particular homo- or heterobifunctional crosslinking agents.

For the purposes of this invention, the term “bifunctional crosslinking agents” stands for compounds which contain two leaving groups that may be the same or different (e.g., —Cl, —Br, —I, -tosyl, -mesyl, —N═C═O, ═O, —C(═O)H, —C(═O)Cl, epoxy). The leaving groups —Cl, —Br, —I, -tosyl, -mesyl, —N═C═O and epoxy are especially advantageous. In particular —Cl, —Br, —I, —N═C═O and epoxy are advantageous.

For the purposes of this invention, the term “leaving group” comprises not only reactive groups, for example, —Cl, —Br, —I, -tosyl or -mesyl, which leave the crosslinking agent as a nucleofuge during the crosslinking reaction, but also those leaving groups, for example, —N═C═O or epoxy, whose atoms do not leave the crosslinking agent during the crosslinking reaction but are converted to other functional groups, for example, —NHC(═O)— or hydroxyalkyl (for the definition of terms, reference is made to: P. Muller. Glossary of Terms Used in Physical Organic Chemistry (IUPAC Recommendations 1994). Pure Appl. Chem., 1994, 66(5), 1077-1184).

In the case when the bifunctional crosslinking agent contains two identical leaving groups, it is referred to in the sense of this invention as a “homobifunctional cross-linking agent” (e.g., 1,2-dihaloethane). For the purposes of this invention, this is referred to as “heterobifunctional crosslinking agent” (e.g., epichlorohydrin) in the case when the two leaving groups of the bifunctional crosslinking agent are different from one another. The two leaving groups, which are the same or different, of the corresponding homo- or heterobifunctional crosslinking agents are preferably selected from the group consisting of —Cl, —Br, —I, -tosyl, -mesyl, —N═C═O, ═O, —C(═O)H, —C(═O)Cl and epoxy. It is especially preferable for the leaving groups to be selected from the group consisting of —Cl, —Br, —I, -tosyl, -mesyl, —N═C═O and epoxy. In particular —Cl, —Br, —I, —N═C═O and epoxy are advantageous.

The term “polyfunctional crosslinking agents” in the sense of this invention stands for compounds containing three or more leaving groups (for example, 4, 5 or 6), which may be the same or different and are preferably selected from the group consisting of —Cl, —Br, —I, -tosyl, -mesyl, —N═C═O, ═O, —C(═O)H, —C(═O)Cl and epoxy. It is especially preferable for the leaving groups to be selected from the group consisting of —Cl, —Br, —I, -tosyl, -mesyl, —N═C═O and epoxy. In particular —Cl, —Br, —I, —N═C═O and epoxy are advantageous.

In the case when the polyfunctional crosslinking agent contains three or more identical leaving groups (for example, 4, 5 or 6), this is referred to in the sense of this invention as a “homopolyfunctional crosslinking agent.” In the case when the polyfunctional crosslinking agent contains at least two different leaving groups, this is referred to as a “heteropolyfunctional crosslinking agent” for the purposes of this description. The two leaving groups, which are the same different, of the corresponding homo- or heterobifunctional crosslinking agents are preferably selected from the group consisting of —Cl, —Br, —I, -tosyl, -mesyl, —N═C═O, ═O, —C(═O)H, —C(═O)Cl and epoxy. It is especially preferable for the leaving groups to be selected from the group consisting of —Cl, —Br, —I, -tosyl, -mesyl, —N═C═O and epoxy. In particular —Cl, —Br, —I, —N═C═O and epoxy are advantageous.

In a preferred embodiment, the crosslinking agent is a C₁₋₆ hydrocarbon, a 3- to 7-membered cyclic hydrocarbon, benzene or a 5- to 7-membered heteroaromatic, each of which may be substituted with two or more leaving groups (e.g., 3, 4, 5 or 6) that are the same or different, where the leaving groups are selected from the group consisting of —Cl, —Br, —I, -tosyl, -mesyl, —N═C═O, ═O, —C(═O)H, —C(═O)Cl and epoxy.

In another preferred embodiment, the crosslinking agent is a C₁₋₆ hydrocarbon or a 3- to 7-membered cyclic hydrocarbon, each of which is substituted with two or more leaving groups (e.g., 3, 4, 5 or 6) that are the same or different, where the leaving groups are selected from the group consisting of —Cl, —Br, —I, -tosyl, -mesyl, —N═C═O, ═O, —C(═O)H, —C(═O)Cl and epoxy.

In the sense of this invention, the term “C₁₋₆ hydrocarbon” comprises acyclic, saturated or unsaturated hydrocarbons, which are branched or linear and contain 1, 2, 3, 4, 5 or 6 carbon atoms. The term “C₁₋₆ hydrocarbon” comprises C₁₋₆-alkanes, C₂₋₆-alkenes and C₂₋₆-alkynes. C₂₋₆-alkenes contain at least one C—C double bond and C₂₋₆-alkyne contain at least one C—C triple bond. C₁₋₆ hydrocarbon is preferably selected from the group consisting of methane, ethane, isopropane, N-propane, isobutane, N-butane, sec-butane, tert-butane, N-pentane, isopentane, neopentane, isohexane, N-hexane, neohexane, 3-methylpentane, 2,3-dimethylbutane, ethene, ethyne, prop-1-ene, isobutene, N-butene, cis-2-butene, trans-2-butene, 1,2-butadiene, 1,3-butadiene, pent-1-ene, cis-pent-2-ene, trans-pent-2-ene, 2-methylbut-1-ene, 2-methylbut-2-ene, 3-methylbut-1-ene, hex-1-ene, hex-2-en and hex-3-en. The C₁₋₆ hydrocarbons are substituted two or more times with the same or different leaving groups, where the leaving groups may be in any possible position on the C₁₋₆ hydrocarbon.

With respect to the crosslinking agent, the term “3- to 7-membered cyclic hydrocarbon” for the purposes of this invention stands for cyclic, saturated or unsaturated (but not aromatic) hydrocarbons containing 3, 4, 5, 6 or 7 carbon atoms. However, one or two carbon atoms may be replaced by the same or different heteroatoms selected from the group consisting of N, O and S. The 3- to 7-membered cyclic hydrocarbon is preferably selected from the group consisting of cyclopropane, cyclobutane, cyclopentane, cyclohexane, cycloheptane, cyclopropene, cyclobutene, cyclopentene, cyclopentadiene, cyclohexene, cyclohexadiene, cycloheptene, cycloheptadiene, cycloheptatriene, pyrroline, pyrrolidine, tetrahydrofuran, pyrazoline, pyrazolidine, imidazoline, imidazolidine, dioxolane, piperidine, N-methylpiperidine, tetrahydropyran, piperazine, N,N-dimethylpiperazine, dioxane, morpholine, N-methylmorpholine, pyrazoline, pyrazolinone and pyranyl. The 3- to 7-membered cyclic hydrocarbons are substituted at least twice with the same or different leaving groups, where the leaving groups may be in any possible position on the 3- to 7-membered cyclic hydrocarbon.

With respect to the crosslinking agent, the aromatic hydrocarbon benzene in the sense of this invention is substituted at least twice (e.g., two, three or four times) with the same of different leaving groups, where the leaving groups may be in any possible position on the benzene. The benzene may optionally also be part of a bicyclic or polycyclic system (e.g., naphthalene).

For the purposes of this invention, the term “5- to 7-membered heteroaromatic” comprises 5-, 6- or 7-membered cyclic aromatics containing at least 1, 2, 3, 4 or 5 heteroatoms, where the heteroatoms may be the same or different and are selected from the group consisting of N, O and S. The heteroaromatic may optionally be part of a bicyclic or polycyclic system. It is preferable for the 5- to 7-membered heteroaromatic to be selected from the group consisting of pyrrole, pyrazole, imidazole, thiophene, furan, oxazole, isoxazole, thiazole, isothiazole, furazan, oxadiazole, pyridine, pyridazine, pyrazine and pyrimidine. The 5- to 7-membered heteroaromatics are substituted two or more times with the same or different leaving groups, where the leaving groups may be in any possible position on the 5- to 7-membered heteroaromatic.

With respect to C₁₋₆ hydrocarbon, the term “substituted at least twice” in the sense of this invention is understood to refer to substitution of two or more hydrogen radicals by at least two leaving groups that are the same or different and are substituted multiple times (e.g., twice) on the same or different carbon atoms of the C₁₋₆ hydrocarbon, for example, substituted twice on the same carbon as on the case di(oxiran-2-yl)methane, or substituted in different positions as in the case of 1,2-dichlorethane. Multiple substitution may be accomplished with the same leaving groups (e.g., 1,2-dichlorethane) or with different leaving groups (e.g., epichlorohydrin).

With respect to the 3- to 7-membered cyclic hydrocarbon, benzene and 5- to 7-membered heteroaromatic, the phrase “substituted at least twice” in the sense of this invention is understood to refer to substitution of two or more hydrogen radicals of the ring systems by at least two leaving groups that are the same or different. Multiple substitution is possible with the same or different leaving groups and on different ring members, or on the same ring members of the ring systems, if possible.

In another preferred embodiment, the crosslinking agent is selected from the group consisting of methane, ethane, isopropane, N-propane, isobutane, N-butane, sec-butane, tert-butane, N-pentane, isopentane, neopentane, isohexane, N-hexane, neohexane, 3-methylpentane, 2,3-dimethylbutane, ethene, ethyne, prop-1-ene, isobutene, N-butene, cis-2-butene, trans-2-butene, 1,2-butadiene, 1,3-butadiene, pent-1-ene, cis-pent-2-ene, trans-pent-2-ene, 2-methylbut-1-ene, 2-methylbut-2-ene, 3-methylbut-1-ene, hex-1-ene, hex-2-en hex-3-ene, cyclopropane, cyclobutane, cyclopentane, cyclohexane, cycloheptane, cyclopropene, cyclobutene, cyclopentene, cyclopentadiene, cyclohexene, cyclohexadiene, cycloheptene, cycloheptadiene, cycloheptatriene, pyrroline, pyrrolidine, tetrahydrofuran, pyrazoline, pyrazolidine, imidazoline, imidazolidine, dioxolane, piperidine, N-methylpiperidine, tetrahydropyran, piperazine, N,N-dimethylpiperazine, dioxane, morpholine, N-methylmorpholine, pyrazoline, pyrazolinone, pyran, benzene, pyrrole, pyrazole, imidazole, thiophene, furan, oxazole, isoxazole, thiazole, isothiazole, furazan, oxadiazole, pyridine, pyridazine, pyrazine and pyrimidine, each of which may be substituted with two or more leaving groups (e.g., 3, 4, 5 or 6) that are the same or different, where the leaving groups are selected from the group consisting of —Cl, —Br, —I, -tosyl, -mesyl, —N═C═O, ═O, —C(═O)H, —C(═O)Cl and epoxy.

In another preferred embodiment, the crosslinking agent is selected from the group consisting of methane, ethane, isopropane, N-propane, isobutane, N-butane, sec-butane, tert-butane, N-pentane, isopentane, neopentane, isohexane, N-hexane, neohexane, 3-methylpentane, 2,3-dimethylbutane, cyclopropane, cyclobutane, cyclopentane, cyclohexane, cycloheptane, tetrahydrofuran, dioxolane, tetrahydropyran, dioxane, benzene, pyrrole, pyrazole, imidazole, thiophene, furan, oxazole, isoxazole, thiazole, isothiazole, furazan, oxadiazole, pyridine, pyridazine, pyrazine and pyrimidine, each of which may be substituted with two or more leaving groups (e.g., 3, 4, 5 or 6) that are the same or different, where the leaving groups are selected from the group consisting of —Cl, —Br, —I, -tosyl, -mesyl, —N═C═O, ═O, —C(═O)H, —C(═O)Cl and epoxy.

In another preferred embodiment, the crosslinking agent is selected from the group consisting of methane, ethane, isopropane, N-propane, isobutane, N-butane, sec-butane, tert-butane, N-pentane, isopentane, neopentane, isohexane, N-hexane, neohexane, 3-methylpentane, 2,3-dimethylbutane, cyclopropane, cyclobutane, cyclopentane, cyclohexane and cycloheptane, each of which may be substituted with two or more leaving groups (e.g., 3, 4, 5 or 6) that are the same or different, where the leaving groups are selected from the group consisting of —Cl, —Br, —I, -tosyl, -mesyl, —N═C═O, ═O, —C(═O)H, —C(═O)Cl and epoxy.

In another preferred embodiment, the crosslinking agent is selected from the group consisting of methane, ethane, isopropane, N-propane, isobutane, N-butane, sec-butane, tert-butane, N-pentane, isopentane, neopentane, isohexane, N-hexane, neohexane, 3-methylpentane, 2,3-dimethylbutane, cyclopropane, cyclobutane, cyclopentane and cyclohexane, each of which may be substituted with 2 or more leaving groups that are the same or different, where the leaving groups are selected from the group consisting of —Cl, —Br, —I, -tosyl, -mesyl, —N═C═O, ═O, —C(═O)H, —C(═O)Cl and epoxy.

In another preferred embodiment, the crosslinking agent is selected from the group consisting of methane, ethane, N-propane, N-butane, N-pentane, N-hexane, cyclopropane, cyclobutane, cyclopentane and cyclohexane each of which may be substituted with two or more leaving groups that are the same or different, where the leaving groups are selected from the group consisting of —Cl, —Br, —I, —N═C═O, ═O, —C(═O)H, —C(═O)Cl and epoxy.

In another preferred embodiment, the crosslinking agent is selected from the group consisting of glyoxal, 1,2-dihaloethane (e.g., 1,2-dichloroethane), 1,3-dihalopropane (e.g., 1,3-dichloropropane), halocarboxylic acid halide [for example, haloacetic acid halide (e.g., chloroacetic acid chloride) or halopropionic acid halide (e.g., chloropropionic acid chloride)], epichlorohydrin, 4-chloro-1,2-epoxybutane, 1,2,3,4-diepoxybutane, tetramethylene diisocyanate and hexamethylene diisocyanate, where halogen and halide, independently of one another, preferably stand for —Cl, —Br or —I, especially preferably for —Cl or —Br and in particular for —Cl.

In another preferred embodiment, the crosslinking agent is selected from the group consisting of glyoxal, epichlorohydrin, 4-chloro-1,2-epoxybutane and 1,2,3,4-diepoxybutane. The crosslinking agents are especially advantageously epichlorohydrin, 4-chloro-1,2-epoxybutane and 1,2,3,4-diepoxybutane.

The crosslinking agent epichlorohydrin is preferred in particular.

For the purposes of this invention, the term “functionalizing agent” stands for compounds containing one or more, preferably only one reactive leaving group (e.g., —Cl, —Br, —I, -tosyl, -mesyl, —C(═O)Cl, epoxy) and which may also contain other functional groups that result in little or no significant crosslinking of the cyclodextrin compounds (e.g., —OH, alkyloxy, —C(═O)OH). Typical functionalizing agents include, for example, alkyl halides, cyclic alkane sultone, carboxylic acid halides, carboxylic acid anhydrides, halocarboxylic acids, halocarboxylic acid esters, (haloalkyl)trialkylammonium salts and oxiranes.

In the sense of this invention, the phrase “result in little or no significant crosslinking of the cyclodextrin compounds” means that preferably <10 mol %, more preferably <5.0 mol %, even more preferably <2.0 mol %, most preferably <1.0 mol % and in particular an analytically non-detectable amount of the cyclodextrin monomers in the inventive copolymers is crosslinked via linkers derived from the functionalizing agents used, based on the total amount of linkers of the inventive copolymers.

However, the functionalizing agent may also be ammonia, a primary, secondary, or tertiary amine, which reacts with the crosslinking agents to form amino and/or ammonium substituents in the inventive compounds. For example, the structural element —CH₂—CH(OH)—CH₂—Cl, which is derived from epichlorohydrin and is bound to a cyclodextrin compound or a cyclodextrin monomer at one end, may react with NH(CH₃)₂ to form —CH₂—CH(OH)—CH₂—N(CH₃)₂.

The functionalizing agent is preferably selected from the group consisting of C₁₋₆-alkyl-Y, C₃₋₇-cycloalkyl-Y, C₃₋₇-cycloalkyl-C(═O)—Y, Y—C₁₋₆-alkyl-C(═O)—OH, Y—C₃₋₇-cycloalkyl-C(═O)—OH, Y—C₁₋₆-alkyl-C(═O)—O—C₁₋₆-alkyl, Y—C₃₋₇-cycloalkyl-C(═O)—O—C₃₋₇-cycloalkyl, [C₁₋₆-alkyl-C(═O)]₂O, [C₃₋₇-cycloalkyl-C(═O)]₂O, dihydrofuran-2,5-dione, furan-2,5-dione, dihydro-2H-pyran-2,6(3H)-dione, oxirane, C₁₋₆-alkyloxirane, C₃₋₇-cycloalkyloxirane, di-C₁₋₆-alkyloxirane, di-C₃₋₇-cycloalkyloxirane, 2-chloroethanol, di-C₁₋₆-alkyl sulfate, di-C₃₋₇-cycloalkyl sulfate, di-C₁₋₆-alkyl phosphorochloridate, di-C₃₋₇-cycloalkyl phosphorochloridate, NH₃, (C₁₋₆-alkyl)NH₂, (C₃₋₇-cycloalkyl)NH₂, (C₁₋₆-alkyl)₂NH, (C₃₋₇-cycloalkyl)₂NH, (C₁₋₆-alkyl)₃N, (C₃₋₇-cycloalkyl)₃N, [(Y—C₁₋₆-alkyl)N(C₁₋₆-alkyl)₃]⁺ halide, 1,3-propane sultone and 1,4-butane sultone; where C₁₋₆-alkyl and C₃₋₇-cycloalkyl, independently of one another, may be unsubstituted or mono- or polysubstituted with the same or different Z radicals, where the additional substituent Z results in little or no significant crosslinking of the cyclodextrin monomers and/or compounds; halide preferably stands for chloride, bromide or iodide, especially preferably for chloride or bromide and in particular for chloride; and the Y radicals stand for leaving groups.

The leaving groups Y, independently of one another, are selected from the group consisting of —Cl, —Br, —I, -mesyl, -tosyl and epoxy. Especially advantageous are —Cl, —Br, -mesyl and -tosyl; in particular —Cl.

The substituents Z, independently of one another, are preferably selected from the group consisting of —OH, ═O, —C(═O)OH, —C(═O)OCH₃, —C(═O)OCH₂CH₃, —NH₂, —NH(CH₃), —N(CH₃)₂, —N(CH₃)₃⁺A⁻, —NH(CH₂CH₃), —N(CH₂CH₃)₂ and —N(CH₂CH₃)₃⁺A⁻; where A⁻ stands for an organic or inorganic anion, which is preferably selected from the group consisting of Cl⁻, Br⁻, I⁻, dihydrogen phosphate, hydrogen phosphate, phosphate, bicarbonate, carbonate, acetate, lactate and citrate or for other physiologically tolerable anions. Especially advantageous are —OH, ═O, —C(═O)OH, —C(═O)OCH₃ and —C(═O)OCH₂CH₃; in particular —OH, ═O and —C(═O)OH.

With respect to the functionalizing agent, the term “C₁₋₆-alkyl” in the sense of this invention comprises acyclic, saturated or unsaturated hydrocarbon radicals, which have 1, 2, 3, 4, 5 or 6 carbon atoms, are branched or linear and may contain a leaving group Y and additional substituents Z, but the additional substituents Z result in little or no significant crosslinking of the cyclodextrin monomers and/or compounds. The leaving group and optional substituents Z may be in any possible position on the C₁₋₆-alkyls. C₁₋₆-Alkyl comprises C₁₋₆-alkanyl, C₂₋₆-alkenyl and C₂₋₆-alkynyl. C₂₋₆-alkenyls have at least one C—C double bond, and C₂₋₆-alkynyls have at least one C—C triple bond, but C₁₋₆-alkanyls are completely saturated.

With respect to the functionalizing agent, C₁₋₆-alkyl is preferably selected from the group consisting of methyl, ethyl, isopropyl, N-propyl, isobutyl, N-butyl, sec-butyl, tert-butyl, N-pentyl, isopentyl, neopentyl, isohexyl, N-hexyl, neohexyl, 3-methylpentyl, 2,3-dimethylbutyl, ethenyl, ethynyl, prop-1-enyl, isobutenyl, N-butenyl, cis-2-butenyl, trans-2-butenyl, 1,2-butadienyl, 1,3-butadienyl, pent-1-enyl, cis-pent-2-enyl, trans-pent-2-enyl, 2-methylbut-1-enyl, 2-methylbut-2-enyl, 3-methylbut-1-enyl, hex-1-enyl, hex-2-enyl and hex-3-enyl. It is especially preferred for the C₁₋₆-alkyl to be selected from the group consisting of methyl, ethyl, isopropyl, N-propyl, isobutyl, N-butyl, sec-butyl, tert-butyl, ethenyl, ethynyl and prop-1-enyl. It is in particular preferred that C₁₋₆-alkyl is selected from the group consisting of methyl, ethyl, isopropyl, N-propyl, isobutyl, N-butyl, sec-butyl and tert-butyl. With respect to the functionalizing agent, the C₁₋₆-alkyl contains a leaving group Y and may be substituted with one or more substituents Z, which may be the same or different.

With respect to the functionalizing agent, the term “C₃₋₇-cycloalkyl” for the purposes of this invention stands for cyclic hydrocarbon radicals with 3, 4, 5, 6 or 7 carbon atoms, which are saturated or unsaturated (but not aromatic), contain a leaving group Y and may contain additional substituents Z, where the additional substituents Z result in little or no significant crosslinking of the cyclodextrin monomers. The leaving group and optionally the substituents Z may be in any possible position on the C₃₋₇-cycloalkyl. The term “C₃₋₇-cycloalkyl” also comprises or unsaturated (but not aromatic) cycloalkyls in which one or two carbon atoms are replaced by a heteroatom N, O or S. The saturated, unsaturated (but not aromatic) C₃₋₇-cycloalkyl may be condensed with one or more saturated, unsaturated or aromatic radicals, for example, cyclohexyl, cyclohexenyl, cyclohexadienyl or benzene (e.g., indoline).

With respect to the functionalizing agent, the C₃₋₇-cycloalkyl radicals, independently of one another, are preferably selected from the group consisting of cyclopropyl, cyclobutyl, cyclopentyl, cyclohexyl, cycloheptyl, cyclopropenyl, cyclobutenyl, cyclopentenyl, cyclopentadienyl, cyclohexenyl, cyclohexadienyl, cycloheptenyl, cycloheptadienyl, cycloheptatrienyl, pyrrolinyl, pyrrolidinyl, tetrahydrofuranyl, pyrazolinyl, pyrazolidinyl, imidazolinyl, imidazolidinyl, dioxolanyl, piperidinyl, N-methylpiperidinyl, tetrahydropyranyl, piperazinyl, N,N-dimethylpiperazinyl, dioxanyl, morpholinyl, N-methylmorpholinyl, quinuclidinyl, pyrazolinyl, pyrazolinonyl, pyranyl, indolinyl, quinolizinyl, chromanyl, isochromanyl, chromenyl, isochromenyl and benzodioxolanyl. It is especially preferable for the C₃₋₇-cycloalkyl to be selected from the group consisting of cyclopropyl, cyclobutyl, cyclopentyl, cyclohexyl, pyrrolinyl, pyrrolidinyl, tetrahydrofuranyl, pyrazolinyl, pyrazolidinyl, imidazolinyl, imidazolidinyl, dioxolanyl, piperidinyl, N-methylpiperidinyl tetrahydropyranyl, piperazinyl, N,N-dimethylpiperazinyl, dioxanyl, morpholinyl, N-methylmorpholinyl, quinuclidinyl, pyrazolinyl, pyrazolinonyl and pyranyl. It is preferred in particular for C₃₋₇-cycloalkyl to be selected from the group consisting of cyclopropyl, cyclobutyl, cyclopentyl and cyclohexyl. With respect to the functionalizing agent, C₃₋₇-cycloalkyl contains a leaving group Y and may be substituted with one or more substituents Z that are the same or different.

In a preferred embodiment, the functionalizing agents are selected from the group consisting of CH₃—Y¹, CH₃—CH₂—Y¹, CH₃—CH₂—CH₂—Y¹, (CH₃)₂CH—Y¹, CH₃—CH₂—CH₂—CH₂—Y¹, (CH₃)₂CH—CH₂—Y¹, CH₃—CH₂—CH(CH₃)—Y¹, (CH₃)₃C—Y¹, CH₂═CH—Y¹, CH₂═CH—CH₂—Y¹, cyclopropyl-Y¹, Y¹—CH₂—C(═O)—OH, Y¹—CH₂—CH₂—C(═O)—OH, Y¹—CH(CH₃)—C(═O)—OH, Y¹—CH₂—C(═O)—O—CH₃, Y¹—CH₂—CH₂—C(═O)—O—CH₃, Y¹—CH(CH₃)—C(═O)—O—CH₃, Y¹—CH₂—C(═O)—O—CH₂CH₃, Y¹—CH₂—CH₂—C(═O)—O—CH₂—CH₃, Y¹—CH(CH₃)—C(═O)—O—CH₂—CH₃, CH₃—C(═O)Y², CH₃—CH₂—C(═O)Y², (CH₃)₂CH—C(═O)Y², CH₃—CH₂—CH₂—C(═O)Y², (CH₃)₃C—C(═O)Y², CH₃—CH₂—CH(CH₃)—C(═O)Y², (CH₃)₂CH—CH₂—C(═O)Y², CH₂═CH—C(═O)Y², CH₂═CH—CH₂—C(═O)Y², cyclopropyl-C(═O)Y², dihydrofuran-2,5-dione, furan-2,5-dione, dihydro-2H-pyran-2,6(3H)-dione, [CH₃—C(═O)]₂O, [CH₃—CH₂—C(═O)]₂O, [CH₃—CH₂—CH₂—C(═O)]₂O, [(CH₃)₂CH—C(═O)]₂O, [CH₃—CH₂—CH₂—CH₂—C(═O)]₂O, [CH₃—CH₂—CH(CH₃)—C(═O)]₂O, [(CH₃)₂CH—CH₂—C(═O)]₂O, [(CH₃)₃C—C(═O)]₂O, [CH₂═CH—C(═O)]₂O, [CH₂═CH—CH₂—C(═O)]₂O, [cyclopropyl-C(═O)]₂O, CH₂═CH—C(═O)—OH, CH₃—CH═CH—C(═O)—OH, (CH₃)₂C═CH—C(═O)—OH, CH₃—CH₂—CH═CH—C(═O)—OH, CH₃—CH═C(CH₃)—C(═O)—OH, CH₂═CH—C(═O)—O—CH₃, CH₃—CH═CH—C(═O)—O—CH₃, (CH₃)₂C═CH—C(═O)—O—CH₃, CH₃—CH₂—CH═CH—C(═O)—O—CH₃, CH₃—CH═C(CH₃)—C(═O)—O—CH₃, CH₂═CH—C(═O)—O—CH₂—CH₃, CH₃—CH═CH—C(═O)—O—CH₂—CH₃, (CH₃)₂C═CH—C(═O)—O—CH₂—CH₃, CH₃—CH₂—CH═CH—C(═O)—O—CH₂—CH₃, CH₃—CH═C(CH₃)—C(═O)O—CH₂—CH₃, oxirane, 2-methyloxirane, 2-ethyloxirane, oxiran-2-yl-methanol, oxirane-2-carboxylic acid, oxirane-2-carboxylic acid methyl ester, oxirane-2-carboxylic acid ethyl ester, 2-(oxiran-2-yl)acetic acid, 2-(oxiran-2-yl)acetic acid methyl ester, 2-(oxiran-2-yl)acetic acid ethyl ester, Cl—CH₂—CH₂—OH, (CH₃—O)₂S(═O)₂, (CH₃—CH₂—O)₂S(═O)₂, (CH₃—O)₂P(═O)Cl, (CH₃—CH₂—O)₂P(═O)Cl, [(CH₃)₂CH—O]₂P(═O) Cl, NH₃, CH₃—NH₂, (CH₃)₂NH, (CH₃)₃N, CH₃—CH₂—NH₂, (CH₃—CH₂)₂NH, (CH₃—CH₂)₃N, [Cl—CH₂—CH₂—N(CH₃)₃]⁺Cl⁻, [Cl—CH₂—CH₂—N(CH₂—CH₃)₃]⁺Cl⁻, 1,3-propane sultone and 1,4-butane sultone; where the Y¹ radicals, independently of one another, preferably stand for —Cl, —Br, —I, -tosyl, -mesyl or epoxy, especially preferably —Cl, —Br or epoxy, and in particular —Cl; and the Y² radicals, independently of one another, preferably stand for —Cl, —Br or —I, especially preferably —Cl or —Br and in particular —Cl.

The inventive copolymers can preferably be obtained by mixing (combining) at least one α-, β- or γ-cyclodextrin compound with one or more crosslinking agent, preferably only one crosslinking agent, preferably in the presence of at least one solvent.

The inventive copolymers can preferably also be obtained by mixing (combining) a mixture containing different α-, different β- or different γ-cyclodextrin compounds or by mixing (combining) a mixture containing α- and β-, α- and γ-, β- and γ- or α-, β- and γ-cyclodextrin compounds, with one or more crosslinking agents, preferably only one crosslinking agent, preferably in the presence of at least one solvent.

In a preferred embodiment, an α-, γ- or γ-cyclodextrin compound or a mixture containing at least two different cyclodextrin compounds of those listed above, optionally in the presence of a solvent, is mixed with at least one, preferably only one crosslinking agent, selected from the group consisting of methane, ethane, isopropane, N-propane, isobutane, N-butane, sec-butane, tert-butane, N-pentane, isopentane, neopentane, isohexane, N-hexane, neohexane, 3-methylpentane, 2,3-dimethylbutane, ethene, ethyne, prop-1-ene, isobutene, n-butene, cis-2-butene, trans-2-butene, 1,2-butadiene, 1,3-butadiene, pent-1-ene, cis-pent-2-ene, trans-pent-2-ene, 2-methylbut-1-ene, 2-methylbut-2-ene, 3-methylbut-1-ene, hex-1-ene, hex-2-en hex-3-ene, cyclopropane, cyclobutane, cyclopentane, cyclohexane, cycloheptane, cyclopropene, cyclobutene, cyclopentene, cyclopentadiene, cyclohexene, cyclohexadiene, cycloheptene, cycloheptadiene, cycloheptatriene, pyrroline, pyrrolidine, tetrahydrofuran, pyrazoline, pyrazolidine, imidazoline, imidazolidine, dioxolane, piperidine, N-methylpiperidine, tetrahydropyran, piperazine, N,N-dimethylpiperazine, dioxane, morpholine, N-methylmorpholine, pyrazoline, pyrazolinone, pyran, benzene, pyrrole, pyrazole, imidazole, thiophene, furan, oxazole, isoxazole, thiazole, isothiazole, furazan, oxadiazole, pyridine, pyridazine, pyrazine and pyrimidine, each of which is substituted with two or more leaving groups (e.g., 3, 4, 5 or 6), which are the same or different, where the leaving groups are selected from the group consisting of —Cl, —Br, —I, -tosyl, -mesyl, —N═C═O, ═O, —C(═O)H, —C(═O)Cl and epoxy.

In a preferred embodiment, an α-, β- or γ-cyclodextrin compound or a mixture containing at least two different cyclodextrin compounds of those listed above, optionally in the presence of a solvent, is mixed with at least one, preferably only one crosslinking agent selected from the group consisting of glyoxal, 1,2-dihaloethane (e.g., 1,2-dihaloethane), 1,3-dihalopropane (e.g., 1,3-dichloropropane), halocarboxylic acid halide, [for example, haloacetic acid halide (e.g., chloroacetic acid chloride) or halopropionic acid halide (e.g., chloropropionic acid chloride)], epichlorohydrin, 4-chloro-1,2-epoxybutane, 1,2,3,4-diepoxybutane, tetramethylene diisocyanate and hexamethylene diisocyanate; where halogen and halide, independently of one another, preferably stand for —Cl, —Br or —I, especially preferably stand for —Cl or —Br and in particular for —Cl.

In another preferred embodiment, an α-, β- or γ-cyclodextrin compound or a mixture containing at least two different cyclodextrin compounds of those listed above, optionally in the presence of a solvent, is mixed with the crosslinking agent epichlorohydrin and optionally an additional crosslinking agent selected from the group consisting of glyoxal, 1,2-dihaloethane (e.g., 1,2-dichloroethane), 1,3-dihalopropane (e.g., 1,3-dichloropropane), halocarboxylic acid halide [for example, haloacetic acid halide (e.g., chloroacetic acid chloride) or halopropionic acid halide (e.g., chloropropionic acid chloride)], 4-chloro-1,2-epoxybutane, 1,2,3,4-diepoxybutane, tetramethylene diisocyanate and hexamethylene diisocyanate; where halogen and halide, independently of one another, preferably stand for —Cl, —Br or —I, especially preferably stand for —Cl or —Br and in particular for —Cl.

In another preferred embodiment, an α-, β- or γ-cyclodextrin compound or a mixture containing at least two different cyclodextrin compounds of those listed above, optionally in the presence of a solvent, is mixed with the crosslinking agent epichlorohydrin and with no other crosslinking agent or with the crosslinking agent 1,2,3,4-diepoxybutane and no other crosslinking agent.

In an especially preferred embodiment, the inventive copolymers can be obtained by mixing: one or at least two cyclodextrin compounds, preferably only one cyclodextrin compound, selected from the group consisting of (glucosyl)-cyclodextrin; (galactosyl)cyclodextrin; (maltosyl)-cyclodextrin; (maltriosyl)-cyclodextrin; (acetyl)-cyclodextrin; (propionyl)-cyclodextrin; (isobutyryl)-cyclodextrin; (butyryl)-cyclodextrin; (pivaloyl)cyclodextrin; (pentanoyl)-cyclodextrin; (hexanoyl)-cyclodextrin; (cyclopropanecarbonyl)-cyclodextrin; (benzoyl)-cyclodextrin; (2-phenylacetyl)-cyclodextrin; (2-carboxyacetyl)-cyclodextrin; (3-carboxypropanoyl)-cyclodextrin; (4-carboxybutanoyl)cyclodextrin; (2-methoxy-2-oxoacetyl)-cyclodextrin; (3-methoxy-3-oxopropanoyl)cyclodextrin; (4-methoxy-4-oxobutanoyl)-cyclodextrin; (2-hydroxyacetyl)-cyclodextrin; (2-hydroxypropanoyl)-cyclodextrin; (3-hydroxypropanoyl)-cyclodextrin; (2-hydroxybutanoyl)-cyclodextrin; (3-hydroxybutanoyl)-cyclodextrin; (4-hydroxybutanoyl)-cyclodextrin; (2-hydroxypentanoyl)-cyclodextrin; (3-hydroxypentanoyl)-cyclodextrin; (4-hydroxypentanoyl)-cyclodextrin; (5-hydroxypentanoyl)-cyclodextrin; (2,3-dihydroxypropanoyl)-cyclodextrin; (2,3-dihydroxybutanoyl)-cyclodextrin; (2,4-dihydroxybutanoyl)-cyclodextrin; (3,4-dihydroxybutanoyl)-cyclodextrin; (2,3-dihydroxypentanoyl)cyclodextrin; (2,4-dihydroxypentanoyl)-cyclodextrin; (2.5-dihydroxypentanoyl)-cyclodextrin; (3,4-dihydroxypentanoyl)-cyclodextrin; (3,5-dihydroxypentanoyl)-cyclodextrin; (4,5-dihydroxypentanoyl)-cyclodextrin; (2,3,4-trihydroxybutanoyl)-cyclodextrin; (2,3,4-trihydroxypentanoyl)-cyclodextrin; (2,3,5-trihydroxypentanoyl)-cyclodextrin; (2,4,5-trihydroxypentanoyl)-cyclodextrin; (3,4,5-trihydroxypentanoyl)-cyclodextrin; (2,3,4,5-tetrahydroxypentanoyl)-cyclodextrin; (2-aminoacetyl)-cyclodextrin; (2-aminopropanoyl)-cyclodextrin; (2-aminobutanoyl)-cyclodextrin; [2-(1H-imidazol-1-yl)acetyl]-cyclodextrin; [3-(1H-imidazol-1-yl)propanoyl]-cyclodextrin, [4-(1H-imidazol-1-yl)butanoyl]-cyclodextrin; [2-(1H-imidazol-1-yl)acetyl]-cyclodextrin; [3-(1H-imidazol-1-yl)propanoyl]-cyclodextrin; [4-(1H-imidazol-1-yl)butanoyl]-cyclodextrin; [2-(pyridin-3-yl)acetyl]-cyclodextrin; [3-(pyridin-3-yl)propanoyl]-cyclodextrin; [4-(pyridin-3-yl)butanoyl]-cyclodextrin; (methyl)-cyclodextrin; (ethyl)-cyclodextrin; (isopropyl)cyclodextrin; (N-propyl)-cyclodextrin; (isobutyl)-cyclodextrin; (N-butyl)-cyclodextrin; (sec-butyl)-cyclodextrin; (tert-butyl)-cyclodextrin; (cyclopropyl)-cyclodextrin; (phenyl)cyclodextrin; (benzyl)-cyclodextrin; (phenethyl)-cyclodextrin; (carboxymethyl)-cyclodextrin; (1-carboxyethyl)-cyclodextrin; (2-carboxyethyl)-cyclodextrin; (1-carboxypropyl)-cyclodextrin; (2-carboxypropyl)-cyclodextrin; (3-carboxypropyl)-cyclodextrin; (1-carboxypropan-2-yl)-cyclodextrin; (4-carboxybutyl)-cyclodextrin; (5-carboxypentyl)cyclodextrin; (5-carboxypentyl)-cyclodextrin; (2-methoxy-2-oxoethyl)-cyclodextrin; (3-methoxy-2-oxopropyl)-cyclodextrin; (4-methoxy-4-oxobutyl)-cyclodextrin; (5-methoxy-5-oxopentyl)-cyclodextrin; (6-methoxy-6-oxohexyl)-cyclodextrin; (2-ethoxy-2-oxoethyl)-cyclodextrin; (3-ethoxy-2-oxopropyl)-cyclodextrin; (4-ethoxy-4-oxobutyl)-cyclodextrin; (5-ethoxy-5-oxopentyl)-cyclodextrin; (6-ethoxy-6-oxohexyl)-cyclodextrin; (3-carboxy-2-hydroxypropyl)-cyclodextrin; (2-hydroxy-4-methoxy-4-oxobutyl)-cyclodextrin; (4-ethoxy-2-hydroxy-4-oxobutyl)-cyclodextrin; (1-carboxy-2-hydroxyethyl)cyclodextrin; (3-hydroxy-1-methoxy-1-oxopropan-2-yl)-cyclodextrin; (1-ethoxy-3-hydroxy-1-oxopropan-2-yl)-cyclodextrin; (1-carboxy-3-hydroxypropan-2-yl)-cyclodextrin; (1-hydroxy-4-methoxy-4-oxobutan-2-yl)-cyclodextrin; (4-ethoxy-1-hydroxy-4-oxobutan-2-yl)-cyclodextrin; (hydroxymethyl)-cyclodextrin; (1-hydroxyethyl)-cyclodextrin; (2-hydroxyethyl)-cyclodextrin; (1-hydroxypropyl)-cyclodextrin; (2-hydroxypropyl)-cyclodextrin; (3-hydroxypropyl)-cyclodextrin; (1-hydroxypropan-2-yl)-cyclodextrin; (2-hydroxypropan-2-yl)-cyclodextrin; (1-hydroxybutyl)-cyclodextrin; (2-hydroxybutyl)-cyclodextrin; (3-hydroxybutyl)-cyclodextrin; (4-hydroxybutyl)-cyclodextrin; (1-hydroxypentyl)-cyclodextrin; (2-hydroxypentyl)-cyclodextrin; (3-hydroxypentyl)-cyclodextrin; (4-hydroxypentyl)-cyclodextrin; (5-hydroxypentyl)-cyclodextrin; (1,2-dihydroxyethyl)-cyclodextrin; (1,2-dihydroxypropyl)-cyclodextrin; (1,3-dihydroxypropyl)-cyclodextrin; (2,3-dihydroxypropyl)-cyclodextrin; (1,2-dihydroxybutyl)-cyclodextrin; (1,3-dihydroxybutyl)-cyclodextrin; (1,4-dihydroxybutyl)-cyclodextrin; (2,3-dihydroxybutyl)-cyclodextrin; (2,4-dihydroxybutyl)-cyclodextrin; (3,4-dihydroxybutyl)cyclodextrin; (1,2-dihydroxypentyl)-cyclodextrin; (1,3-dihydroxypentyl)-cyclodextrin; (1,4-dihydroxypentyl)-cyclodextrin; (1,5-dihydroxypentyl)-cyclodextrin; (2,3-dihydroxypentyl)-cyclodextrin; (2,4-dihydroxypentyl)-cyclodextrin; (2.5-dihydroxypentyl)-cyclodextrin; (3,4-dihydroxypentyl)-cyclodextrin; (3,5-dihydroxypentyl)-cyclodextrin; (4,5-dihydroxypentyl)-cyclodextrin; (1,2,3-trihydroxypropyl)-cyclodextrin; (1,2,3-trihydroxybutyl)-cyclodextrin; (1,2,4-trihydroxybutyl)-cyclodextrin; (1,3,4-trihydroxybutyl)-cyclodextrin; (2,3,4-trihydroxybutyl)-cyclodextrin; (1,2,3-trihydroxypentyl)-cyclodextrin; (1,2,4-trihydroxypentyl)-cyclodextrin; (1,2,5-trihydroxypentyl)-cyclodextrin; (1,3,4-trihydroxypentyl)-cyclodextrin; (1,3,5-trihydroxypentyl)-cyclodextrin; (1,4,5-trihydroxypentyl)-cyclodextrin; (2,3,4-trihydroxypentyl)-cyclodextrin; (2,3,5-trihydroxypentyl)cyclodextrin; (2,4,5-trihydroxypentyl)-cyclodextrin; (3,4,5-trihydroxypentyl)-cyclodextrin; (1,2,3,4-tetrahydroxybutyl)-cyclodextrin; (1,2,3,4-tetrahydroxypentyl)-cyclodextrin; (1,2,4,5-tetrahydroxypentyl)-cyclodextrin, (2,3,4,5-tetrahydroxypentyl)-cyclodextrin; (1,2,3,4,5-pentahydroxypentyl)-cyclodextrin; [(sulfoxy)methyl]-cyclodextrin; [2-(sulfoxy)ethyl]-cyclodextrin; [3-(sulfoxy)propyl]-cyclodextrin; [4-(sulfoxy)butyl]-cyclodextrin; [(phosphonooxy)methyl]-cyclodextrin; [2-(phosphonooxy)ethyl]-cyclodextrin; [3-(phosphonooxy)propyl]-cyclodextrin; [4-(phosphonooxy)butyl]-cyclodextrin; [4-(phosphonooxy)butyl]-cyclodextrin; (sulfomethyl)-cyclodextrin; (2-sulfoethyl)-cyclodextrin; (3-sulfopropyl)-cyclodextrin; (4-sulfobutyl)-cyclodextrin; (5-sulfopentyl)-cyclodextrin; (6-sulfohexyl)-cyclodextrin; (phosphonomethyl)-cyclodextrin; (2-phosphonoethyl)-cyclodextrin; (3-phosphonopropyl)-cyclodextrin; (4-phosphonobutyl)-cyclodextrin; (5-phosphonopentyl)-cyclodextrin; (6-phosphonohexyl)-cyclodextrin; (2-phosphonovinyl)-cyclodextrin; (3-phosphonoallyl)-cyclodextrin; (4-phosphonobut-3-enyl)cyclodextrin; (5-phosphonopent-4-enyl)-cyclodextrin; (6-phosphonohex-5-enyl)-cyclodextrin; (aminomethyl)-cyclodextrin; (2-aminoethyl)-cyclodextrin; (3-aminopropyl)cyclodextrin; (4-aminobutyl)-cyclodextrin; (5-aminopentyl)-cyclodextrin; (6-aminohexyl)-cyclodextrin; [(N,N-dimethylamino)methyl]-cyclodextrin; [2-(N,N-dimethylamino)ethyl]-cyclodextrin; [3-(N,N-dimethylamino)propyl]-cyclodextrin; [4-(N,N-dimethylamino)butyl]-cyclodextrin; [5-(N,N-dimethylamino)pentyl]-cyclodextrin; [6-(N,N-dimethylamino) hexyl]-cyclodextrin; [(N,N-diethylamino)methyl]-cyclodextrin; [2-(N,N-diethylamino)ethyl]-cyclodextrin; [3-(N,N-diethylamino)propyl]-cyclodextrin; [4-(N,N-diethylamino)butyl]-cyclodextrin; [5-(N,N-diethylamino)pentyl]-cyclodextrin; [6-(N,N-diethylamino)hexyl]-cyclodextrin; [(trimethylammonio)methyl]-cyclodextrin chloride; [2-(trimethylammonio)ethyl]-cyclodextrin chloride; [3-(trimethylammonio)propyl]-cyclodextrin chloride; [4-(trimethylammonio)butyl]-cyclodextrin chloride; [5-(trimethylammonio)pentyl]-cyclodextrin chloride; [6-(trimethylammonio)hexyl]-cyclodextrin chloride; [(triethylammonio)methyl]-cyclodextrin chloride; [2-(triethylammonio)ethyl]-cyclodextrin chloride; [3-(triethylammonio)propyl]-cyclodextrin chloride; [4-(triethylammonio)butyl]-cyclodextrin chloride; [5-(triethylammonio)pentyl]-cyclodextrin chloride; [6-(triethylammonio)hexyl]-cyclodextrin chloride; [(1H-imidazol-1-yl)methyl]-cyclodextrin; [2-(1H-imidazol-1-yl)ethyl]-cyclodextrin; [3-(1H-imidazol-1-yl)propyl]-cyclodextrin; [4-(1H-imidazol-1-yl)butyl]-cyclodextrin; (pyridin-3-ylmethyl)cyclodextrin; [2-(pyridin-3-yl)ethyl]-cyclodextrin; [3-(pyridin-3-yl)propyl]-cyclodextrin; [4-(pyridin-3-yl)butyl]-cyclodextrin; [β-D-glucopyranosyloxyuronic acid) methyl]-cyclodextrin; [2-(β-D-glucopyranosyloxyuronic acid) ethyl]-cyclodextrin; [3-β-D-glucopyranosyloxyuronic acid)propyl]-cyclodextrin; [4-(β-D-glucopyranosyloxyuronic acid) butyl]-cyclodextrin; [5-(β-D-glucopyranosyloxyuronic acid)pentyl]-cyclodextrin; and [6-(β-D-glucopyranosyloxyuronic acid)hexyl]-cyclodextrin; where cyclodextrin for α-, β- or γ-cyclodextrin, preferably α- or β-cyclodextrin and in particular for β-cyclodextrin;

with one or at least two different crosslinking agents, preferably only one crosslinking agent selected from the group consisting of epichlorohydrin, 4-chloro-1,2-epoxybutane, 1,2,3,4-diepoxybutane, tetramethylene diisocyanate and hexamethylene diisocyanate; where the crosslinking agent epichlorohydrin is preferred in particular;
each optionally in the presence of at least one solvent; where
the term “cyclodextrin” stands for α-, β- or γ-cyclodextrin, preferably for α- or β-cyclodextrin and in particular for β-cyclodextrin.

In another especially preferred embodiment, (carboxymethyl)-cyclodextrin, (3-sulfopropyl)-cyclodextrin or (4-sulfobutyl)-cyclodextrin is mixed with epichlorohydrin, preferably in the presence of a solvent.

With respect to the α-cyclodextrin compounds, the total degree of substitution is preferably 0<TDS <1.0 <TDS <2.0 <TDS <3 or 0<TDS <4; more preferably 0<TDS <5.0 <TDS <6.0 <TDS <7 or 0<TDS <8; even more preferably 0<TDS <9.0 <TDS <10.0 <TDS <11 or 0<TDS <12; most preferably 0<TDS <13.0 <TDS <14.0 <TDS <15 or 0<TDS <16; and in particular 0<TDS <17 or 0<TDS <18.

With respect to the β-cyclodextrin compound, the total degree of substitution is preferably 0<TDS <1.0 <TDS <2.0 <TDS <3.0 <TDS <4 or 0<TDS <5; more preferably 0<TDS <6.0 <TDS <7.0 <TDS <8.0 <TDS <9 or 0<TDS <10; even more preferably 0<TDS <11.0 <TDS <12.0 <TDS <13.0 <TDS <14 or 0<TDS <15; most preferably 0<TDS <16.0 <TDS <17.0 <TDS <18 or 0<TDS <19; and in particular 0 <TDS <20 or 0<TDS <21.

With respect to the γ-cyclodextrin compound, the total degree of substitution is preferably 0<TDS <1.0 <TDS <2.0 <TDS <3.0 <TDS <4.0 <TDS <5 or 0<TDS <6; more preferably 0<TDS <7.0 <TDS <8.0 <TDS <9.0 <TDS <10.0 <TDS <11 or 0 <TDS <12; even more preferably 0<TDS <13.0 <TDS <14.0 <TDS <15.0 <TDS <16.0 <TDS <17 or 0<TDS <18; most preferably 0<TDS <19.0 <TDS <20.0 <TDS <21 or 0<TDS <22; and in particular 0<TDS <23 or 0<TDS <24.

Mixing of the cyclodextrin compounds with crosslinking agents may be performed in aqueous or organic solvents. They are preferably mixed in water, dimethyl sulfoxide (DMSO), dimethylformamide (DMF), N-methyl-2-pyrrolidone (NMP) or mixtures thereof. The reaction may also be carried out in anhydrous DMSO, anhydrous DMF, anhydrous NMP or mixtures thereof preferably anhydrous conditions. Especially advantageous solvents include water, DMSO and DMF as well as mixtures thereof, in particular water or DMF and mixtures thereof.

The mixing is preferably performed in an alkaline medium, usually at a pH of >7.

The mixing is preferably performed at 7.5≦pH≦14, more preferably 7.5≦pH≦13, even more preferably 8≦pH≦13, most preferably 8≦pH≦12.5 and in particular at 9≦pH≦12.

The pH of the reaction solution or dispersion is preferably adjusted using bases such as basic alkali metal salts, basic alkaline earth metal salts, hydrides, alkoxides or organic bases such as, for example, pyridine or diisopropylamine. Sodium hydroxide, sodium bicarbonate, sodium carbonate, potassium hydroxide, potassium hydroxide, potassium bicarbonate, potassium carbonate, sodium hydride, potassium hydride, sodium methanolate, potassium tert-butoxide, pyridine and diisopropylamine are especially advantageous.

Since the copolymerization and/or crosslinking of the cyclodextrin compounds take place under basic reaction conditions, substituents which are not stable in an alkaline medium may be structurally altered. For example, the ester group of the cyclodextrin compound (2-methoxy-2-oxoethyl)-cyclodextrin may be cleaved to yield a carboxyl group or a carboxylate group, such that the starting compound (2-methoxy-2-oxoethyl)-cyclodextrin is converted to (carboxymethyl)-cyclodextrin. This conversion may be complete or incomplete, based on the total number of ester groups. Consequently, in the corresponding inventive copolymer, all of the ester groups or only a portion thereof may be converted to carboxyl groups.

In a preferred embodiment, the cyclodextrin compounds are dissolved or dispersed in a solvent or solvent mixture and then the crosslinking agent is dissolved or dispersed therein, preferably while stirring the dispersion or solution. The crosslinking agent may be added all at once or successively in portions. The crosslinking agents may be dissolved or dispersed in a solvent (e.g., water, DMF, DMSO, NMP) before the addition and then added to the reaction mixture as a solution or dispersion all at once or successively in portions or added by drops by means of a dropping funnel, pipette, or syringe. The addition or dropwise addition may be performed with the help of a syringe pump.

In another preferred embodiment, the crosslinking agents are dissolved or dispersed in a solvent or solvent mixture and then the cyclodextrin compounds are dissolved or dispersed in the solution or dispersion, preferably while stirring the solution or dispersion. The cyclodextrin compounds may be added all at once or successively in portions. The cyclodextrin compounds may be dissolved or dispersed in a solvent before being added and then added as a solution or dispersion to the reaction mixture all at once or successively in portions or added by drops by means of a dropping funnel, pipette or syringe. The addition or dropwise addition may be performed with the help of a syringe pump.

In another preferred embodiment, the crosslinking agents and cyclodextrin compounds are dissolved or dispersed simultaneously in a solvent or solvent mixture.

In mixing, there is a sudden and unwanted increase in the reaction temperature. It may therefore be advantageous to optionally cool the reaction mixture to ensure an essentially constant reaction temperature. The cooling of the reaction mixture may be accomplished via an ice bath or a water bath.

The reaction mixture may be alkalized before, during or after the addition of the crosslinking agent, before, during or after the addition of the cyclodextrin compounds or before, during or after the addition of a mixture thereof.

The alkaline pH is preferably measured with the help of pH paper or a pH meter. In a preferred embodiment, the pH value is measured continuously or in certain intervals during the reaction. If the pH drops below a certain value (e.g., pH<8 or <7.5), an alkaline pH may be restored in the reaction mixture by adding a base.

The reaction preferably takes place at a reaction temperature (temperature of the reaction mixture) of 5.0 to 90° C., more preferably 10 to 75° C., even more preferably 15 to 50° C., most preferably 15 to 30° C. and in particular 20 to 25° C. After mixing the cyclodextrin compounds with the crosslinking agents, optionally in presence of a solvent or solvent mixture, the temperature of the reaction mixture is preferably set at 5 to 90° C., more preferably at 10 to 75° C., even more preferably at 15 to 50° C., most preferably at 15 to 30° C. and in particular at 20 to 25° C.

In a preferred embodiment, a total substance quantity of ≧0.5 mol, ≧1.0 mol, ≧2.0 mol or ≧3.0 mol, more preferably ≧4.0 mol, mol or ≧6.0 mol, even more preferably ≧7.0 mol, ≧8.0 mol or ≧9.0 mol, most preferably ≧10 mol, ≧15 mol or ≧20 mol, and in particular ≧25 mol crosslinking agent, based on a total substance quantity of 1.0 mol cyclodextrin compounds is used in the reaction mixture (equivalents).

In another preferred embodiment, the ratio of the total substance quantity of cross-linking agent to the total substance quantity of cyclodextrin compounds is more preferably ≧2:1, even more preferably ≧3:1, most preferably ≧5:1 and in particular ≧10:1.

The reaction (crosslinking reaction) may be followed by suitable analytical processes. For example, random samples of the reaction mixture may be taken at certain points in time and analyzed by LC/MS or NMR. The reaction product present in the reaction mixture at a certain point in time can be determined in this way. If the random sample analysis reveals that the desired reaction product is present, the reaction may be terminated and the desired reaction product isolated and/or worked up.

To stop the reaction, the reaction mixture is preferably neutralized, i.e., the pH of the reaction solution or dispersion is preferably set at 6.5≦pH≦7.5, especially preferably at 6.8≦pH≦7.2. The mixture is preferably neutralized with organic acids (e.g., trifluoroacetic acid, acetic acid) or inorganic acids (e.g., hydrochloric acid, sulfuric acid, nitric acid).

In another preferred embodiment, the reaction mixture is dialyzed after the reaction and/or neutralization, preferably dialyzed in a salt-free process, where the dialysis membrane used in this dialysis process has an MWCO (“molecular weight cut-off”) of preferably 0.50 kDa or 1.0 kDa, more preferably of 2.0 kDa or 3.0 kDa, even more preferably of 4.0 kDa or 5.0 kDa, most preferably of 10 kDa and in particular of ≧15 kDa.

The salt-free-dialyzed solution preferably contains a residual amount of ≦10 wt %, more preferably ≦5.0 wt %, even more preferably ≦2.5 wt % and in particular ≦1.0 wt % of compounds with a molecular weight of <1.5 kDa, based on the total mass of the inventive copolymer.

The reaction mixture and/or the essentially salt-free dialysis solution may be concentrated, preferably with the help of a rotary evaporator before further purification and/or isolation. The reaction mixture may also be concentrated prior to dialysis, preferably salt-free dialysis.

The isolation or workup of the inventive copolymers is preferably performed by ultrafiltration, column chromatography (e.g., on silica gel or activated carbon), preparative HPLC or recrystallization. The resulting inventive copolymer can be lyophilized.

In a preferred embodiment, the substance quantity proportion of the linkers contained in the inventive copolymer is 20 to 95 mol %, more preferably 25 to 80 mol %, even more preferably 30 to 70 mol %, most preferably 30 to 60 mol % and in particular 30 to 50 mol %, based on the total substance quantity of monomers contained in the inventive copolymer.

The substance quantity proportion of the cyclodextrin monomers contained in the inventive copolymer preferably amounts to 5.0 to 80 mol %, more preferably 10 to 75 mol %, even more preferably 20 to 70 mol %, most preferably 30 to 70 mol % and in particular 40 to 70 mol %, based on the total substance quantity of monomers contained in the inventive copolymer.

In another preferred embodiment, the sum of the substance quantity proportion of the linkers and the substance quantity proportion of the cyclodextrin monomers ≧25 mol %, ≧30 mol % or ≧40 mol %, more preferably ≧50 mol % or ≧60 mol %, even more preferably ≧70 mol % or ≧80 mol %, most preferably ≧90 mol % or ≧95 mol %, and in particular 100 mol %, based on the total substance quantity of monomers present in the inventive copolymer.

The water solubility of the inventive copolymers at 23° C. is preferably ≧5.0 g l⁻¹, ≧10 g l⁻¹, ≧25 g l⁻¹ or ≧50 g l⁻¹, more preferably ≧75 g l⁻¹, ≧100 g l⁻¹, ≧125 g l⁻¹ or ≧150 g l⁻¹, even more preferably ≧175 g l⁻¹, ≧200 g l⁻¹, ≧225 g l⁻¹ or ≧250 g l⁻¹, most preferably ≧300 g l⁻¹ or ≧400 g l⁻¹ and in particular ≧500 g l⁻¹.

The osmolality of a 50 mM aqueous solution of inventive copolymer is preferably ≧50 mosm kg⁻¹, ≧75 mosm kg⁻¹ or ≧100 mosm kg⁻¹, more preferably ≧125 mosm kg⁻¹, ≧150 mosm kg⁻¹ or ≧175 mosm kg⁻¹, even more preferably ≧200 mosm kg⁻¹ or ≧225 mosm kg⁻¹, most preferably ≧250 mosm kg⁻¹ or ≧275 mosm kg⁻¹ and in particular ≧300 mosm kg⁻¹.

The degree of polymerization of the inventive copolymers is preferably ≧2.0 or ≧3.0, more preferably ≧4.0 or ≧5.0, even more preferably ≧6.0 or ≧7.0, most preferably ≧8.0 or ≧9.0 and in particular ≧10.

The average molecular weight of the inventive copolymers is preferably ≧2.0 kDa, more preferably ≧2.5 kDa, even more preferably ≧3.0 kDa, most preferably ≧4.0 kDa and in particular ≧5.0 kDa. The average molecular weight is preferably measured with the help of a membrane osmometer (distilled water, 23° C.).

In another preferred embodiment, the average molecular weight (M) of the inventive polymers is preferably 2.0≦M≦100 kDa, more preferably 2.0≦M≦50 kDa, even more preferably 2.0≦M≦25 kDa, most preferably 2.0≦M≦15 kDa and in particular 2.0≦M≦10 kDa.

In another especially preferred embodiment, the inventive copolymer, which can be obtained by mixing at least one cyclodextrin compound with at least one crosslinking agent, preferably has a degree of polymerization of ≧2.0 or ≧3.0, more preferably ≧4.0 or ≧5.0, even more preferably ≧6.0 or ≧7.0, most preferably ≧8.0 or ≧9.0 and in particular ≧10.

In another preferred embodiment, the average particle diameter of the inventive copolymers, which can be obtained by mixing at least one cyclodextrin compound with at least one crosslinking agent, is ≦1000 μm, ≦900 μm or ≦800 μm, more preferably ≦700 μm, ≦600 μm or ≦500 μm, even more preferably ≦400 μm, ≦300 μm or ≦200 μm, most preferably ≦100 μm, ≦75 μm or ≦50 μm and in particular ≦25 μm.

The inventive copolymers can preferably be obtained by mixing (combining) at least one α-, β- or γ-cyclodextrin compound with one or more crosslinking agents, preferably one crosslinking agent and one or more functionalizing agents, preferably only one functionalizing agent, preferably in the presence of at least one solvent.

The inventive copolymers can preferably also be obtained by mixing (combining) a mixture containing various α-cyclodextrin compounds, various β-cyclodextrin compounds or various γ-cyclodextrin compounds or by mixing (combining) a mixture containing α- and β-, α- and γ-, β- and γ- or α-, β- and γ-cyclodextrin compounds with one or more crosslinking agents, preferably only one crosslinking agent and one or more functionalizing agents, preferably only one functionalizing agent, preferably in the presence of at least one solvent.

In a preferred embodiment, at least one, preferably only one cyclodextrin compound is mixed with at least one, preferably only one crosslinking agent and at least one, preferably only one functionalizing agent, optionally in the presence of at least one solvent; wherein the crosslinking agent is selected from the group consisting of methane, ethane, isopropane, N-propane, isobutane, N-butane, sec-butane, tert-butane, N-pentane, isopentane, neopentane, isohexane, N-hexane, neohexane, 3-methylpentane, 2,3-dimethylbutane, ethene, ethyne, prop-1-ene, isobutene, N-butene, cis-2-butene, trans-2-butene, 1,2-butadiene, 1,3-butadiene, pent-1-ene, cis-pent-2-ene, trans-pent-2-ene, 2-methylbut-1-ene, 2-methylbut-2-ene, 3-methylbut-1-ene, hex-1-ene, hex-2-ene, hex-3-ene, cyclopropane, cyclobutane, cyclopentane, cyclohexane, cycloheptane, cyclopropene, cyclobutene, cyclopentene, cyclopentadiene, cyclohexene, cyclohexadiene, cycloheptene, cycloheptadiene, cycloheptatriene, pyrroline, pyrrolidine, tetrahydrofuran, pyrazoline, pyrazolidine, imidazoline, imidazolidine, dioxolane, piperidine, N-methylpiperidine, tetrahydropyran, piperazine, N,N-dimethylpiperazine, dioxane, morpholine, N-methylmorpholine, pyrazoline, pyrazolinone, pyran, benzene, pyrrole, pyrazole, imidazole, thiophene, furan, oxazole, isoxazole, thiazole, isothiazole, furazan, oxadiazole, pyridine, pyridazine, pyrazine and pyrimidine, each of which is substituted with two or more leaving groups (e.g., 3, 4, 5 or 6), which are the same or different, the leaving groups being selected from the group consisting of —Cl, —Br, —I, -tosyl, -mesyl, —N═C═O, ═O, —C(═O)H, —C(═O)Cl and epoxy; and

the functionalizing agent is selected from the group consisting of C₁₋₆-alkyl-Y, C₃₋₇-cycloalkyl-Y, C₁₋₆-alkyl-C(═O)—Y, C₃₋₇-cycloalkyl-C(═O)—Y, Y—C₁₋₆-alkyl-C(═O)—OH, Y—C₃₋₇-cycloalkyl-C(═O)—OH, Y—C₁₋₆-alkyl-C(═O)—O—C₁₋₆-alkyl, Y—C₃₋₇-cycloalkyl-C(═O)—O—C₃₋₇-cycloalkyl, [C₁₋₆-alkyl-C(═O)]₂O, [C₃₋₇-cycloalkyl-C(═O)]₂O, dihydrofuran-2,5-dione, furan-2,5-dione, dihydro-2H-pyran-2,6(3H)-dione, oxirane, C₁₋₆-alkyloxirane, C₃₋₇-cycloalkyloxirane, di-C₁₋₆-alkyloxirane, di-C₃₋₇-cycloalkyloxirane, 2-chloroethanol, di-C₁₋₆-alkyl sulfate, di-C₃₋₇-cycloalkyl sulfate, di-C₁₋₆-alkyl phosphorochloridate, di-C₃₋₇-cycloalkyl phosphorochloridate, NH₃, (C₁₋₆-alkyl)NH₂, (C₃₋₇-cycloalkyl)NH₂, (C₁₋₆-alkyl)₂NH, (C₃₋₇-cycloalkyl)₂NH, (C₁₋₆-alkyl)₃N, (C₃₋₇-cycloalkyl)₃N, [(Y—C₁₋₆-alkyl)N(C₁₋₆-alkyl)₃]⁺ halide, 1,3-propane sultone and 1,4-butane sultone; where C₁₋₆-alkyl and C₃₋₇-cycloalkyl, independently of one another, may be unsubstituted or mono- or polysubstituted with the same or different Z radicals; where the additional substituents Z result in little or no significant crosslinking of the cyclodextrin monomers; halide preferably stands for chloride, bromide or iodide, especially preferably for chloride or bromide and in particular for chloride, and the Y radicals stand for leaving groups, where the leaving groups Y, independently of one another, are preferably selected from the group consisting of —Cl, —Br, —I, -mesyl, -tosyl and epoxy, especially preferably selected from the group consisting of —Cl, —Br, —I, -mesyl and -tosyl, and in particular for —Cl, and the substituents Z, independently of one another, are preferably selected from the group consisting of —OH, ═O, —C(═O)OH, —C(═O)OCH₃, —C(═O)OCH₂CH₃, —NH₂, —NH(CH₃), —N(CH₃)₂, —N(CH₃)₃⁺A⁻, —NH(CH₂CH₃), —N(CH₂CH₃)₂ and —N(CH₂CH₃)₃⁺A⁻, especially preferably from the group consisting of —OH, ═O, —C(═O)OH, —C(═O)OCH₃ and —C(═O)OCH₂CH₃, and in particular selected from the group consisting of —OH, ═O and —C(═O)OH, where A⁻ stands for an organic or inorganic anion, which is preferably selected from the group consisting of Cl⁻, Br⁻, I⁻, dihydrogen phosphate, hydrogen phosphate, phosphate, bicarbonate, carbonate, acetate, lactate and citrate or for other physiologically tolerable anions.

In a preferred embodiment, at least one, preferably only one cyclodextrin compound is mixed with at least one, preferably only one crosslinking agent and at least one, preferably only one functionalizing agent, optionally in the presence of at least one solvent; wherein the crosslinking agent is selected from the group consisting of glyoxal, 1,2-dihaloethane (e.g., 1,2-dichloroethane), 1,3-dihalopropane (e.g., 1,3-dichloropropane), halocarboxylic acid halide [for example, haloacetic acid halide (e.g., chloroacetic acid chloride) or halopropionic acid halide (e.g., chloropropionic acid chloride)], epichlorohydrin, 4-chloro-1,2-epoxybutane, 1,2,3,4-diepoxybutane, tetramethylene diisocyanate and hexamethylene diisocyanate; where halogen and halide, independently of one another, preferably stand for —Cl, —Br or —I, especially preferably —Cl or —Br and in particular —Cl; and the functionalizing agent is selected from the group consisting of C₁₋₆-alkyl-Y, cycloalkyl-Y, C₁₋₆-alkyl-C(═O)—Y, C₃₋₇-cycloalkyl-C(═O)—Y, Y—C₁₋₆-alkyl-C(═O)—OH, Y—C₃₋₇-cycloalkyl-C(═O)—OH, Y—C₁₋₆-alkyl-C(═O)—O—C₁₋₆-alkyl, Y—C₃₋₇-cycloalkyl-C(═O)—O—C₃₋₇-cycloalkyl, [C₁₋₆-alkyl-C(═O)]₂O, [C₃₋₇-cycloalkyl-C(═O)]₂O, dihydrofuran-2,5-dione, furan-2,5-dione, dihydro-2H-pyran-2,6(3H)-dione, oxirane, C₁₋₆-alkyloxirane, C₃₋₇-cycloalkyloxirane, di-C₁₋₆-alkyloxirane, di-C₃₋₇-cycloalkyloxirane, 2-chloroethanol, di-C₁₋₆-alkyl sulfate, di-C₃₋₇-cycloalkyl sulfate, di-C₁₋₆-alkyl phosphorochloridate, di-C₃₋₇-cycloalkyl phosphorochloridate, NH₃, (C₁₋₆-alkyl)NH₂, (C₃₋₇-cycloalkyl)NH₂, (C₁₋₆-alkyl)₂NH, (C₃₋₇-cycloalkyl)₂NH, (C₁₋₆-alkyl)₃N, (C₃₋₇-cycloalkyl)₃N, [(Y—C₁₋₆-alkyl)N (C₁₋₆-alkyl)₃]⁺ halide, 1,3-propane sultone and 1,4-butane sultone; where C₁₋₆-alkyl and C₃₋₇-cycloalkyl, independently of one another, may be unsubstituted or mono- or polysubstituted with the same or different Z radicals; where the additional substituents Z result in little or no significant crosslinking of the cyclodextrin monomers; halide preferably stands for chloride, bromide or iodide, especially preferably chloride or bromide and in particular stands for chloride; and the Y radicals stand for leaving groups; where the leaving groups Y, independently of one another, are preferably selected from the group consisting of —Cl, —Br, —I, -mesyl, -tosyl and epoxy, especially preferably selected from the group consisting of —Cl, —Br, —I, -mesyl and -tosyl, and in particular —Cl; and the substituents Z, independently of one another, are preferably selected from the group consisting of —OH, ═O, —C(═O)OH, —C(═O)OCH₃, —C(═O)—OCH₂CH₃, —NH₂, —NH(CH₃), —N(CH₃)₂, —N(CH₃)₃⁺A⁻, —NH(CH₂CH₃), —N(CH₂CH₃)₂ and —N(CH₂CH₃)₃⁺A⁻, especially preferably from the group consisting of —OH, ═O, —C(═O)OH, —C(═O)OCH₃ and —C(═O)OCH₂CH₃, and in particular from the group consisting of —OH, ═O and —C(═O)OH, where A⁻ stands for an organic or inorganic anion, which is preferably selected from the group consisting of Cl⁻, Br⁻, I⁻, dihydrogen phosphate, hydrogen phosphate, phosphate, bicarbonate, carbonate, acetate, lactate and citrate or for other physiologically tolerable anions.

In another preferred embodiment, at least one cyclodextrin compound, preferably only one cyclodextrin compound, is mixed with at least one, preferably only one crosslinking agent, and at least one, preferably only one functionalizing agent, optionally in the presence of at least one solvent; wherein

the crosslinking agent is preferably selected from the group consisting of glyoxal, 1,2-dihaloethane (e.g., 1,2-dichloroethane), 1,3-dihalopropane (e.g., 1,3-dichloropropane), halocarboxylic acid halide [for example, haloacetic acid halide (e.g., chloroacetic acid chloride), halopropionic acid halide (e.g., chloropropionic acid chloride)], epichlorohydrin, 4-chloro-1,2-epoxybutane, 1,2,3,4-diepoxybutane, tetramethylene diisocyanate and hexamethylene diisocyanate, where halogen and halide, independently of one another, preferably stand for —Cl, —Br or —I, especially preferably stand for —Cl or —Br and in particular for —Cl; and

the functionalizing agent is selected from the group consisting of CH₃—Y¹, CH₃—CH₂—Y¹, CH₃—CH₂—CH₂—Y¹, (CH₃)₂CH—Y¹, CH₃—CH₂—CH₂—CH₂—Y¹, (CH₃)₂CH—CH₂—Y¹, CH₃—CH₂—CH(CH₃)—Y¹, (CH₃)₃C—Y¹, CH₂═CH—Y¹, CH₂═CH—CH₂—Y¹, cyclopropyl-Y¹, Y¹—CH₂—C(═O)—OH, Y¹—CH₂—CH₂—C(═O)—OH, Y¹—CH(CH₃)—C(═O)—OH, Y¹—CH₂—C(═O)—O—CH₃, Y¹—CH₂—CH₂—C(═O)—O—CH₃, Y¹—CH(CH₃)—C(═O)—O—CH₃, Y¹—CH₂—C(═O)—O—CH₂CH₃, Y¹—CH₂—CH₂—C(═O)—O—CH₂—CH₃, Y¹—CH(CH₃)—C(═O)—O—CH₂—CH₃, CH₃—C(═O)Y², CH₃—CH₂—C(═O)Y², (CH₃)₂CH—C(═O)Y², CH₃—CH₂—CH₂—C(═O)Y², (CH₃)₃C—C(═O)Y², CH₃—CH₂—CH(CH₃)—C(═O)Y², (CH₃)₂CH—CH₂—C(═O)Y², CH₂═CH—C(═O)Y², CH₂═CH—CH₂—C(═O)Y², cyclopropyl-C(═O)Y², dihydrofuran-2,5-dione, furan-2,5-dione, dihydro-2H-pyran-2,6(3H)-dione, [CH₃—C(═O)]₂O, [CH₃—CH₂—C(═O)]₂O, [CH₃—CH₂—CH₂—C(═O)]₂O, [(CH₃)₂CH—C(═O)]₂O, [CH₃—CH₂—CH₂—CH₂—C(═O)]₂O, [CH₃—CH₂—CH(CH₃)—C(═O)]₂O, [(CH₃)₂CH—CH₂—C(═O)]₂O, [(CH₃)₃C—C(═O)]₂O, [CH₂═CH—C(═O)]₂O, [CH₂═CH—CH₂—C(═O)]₂O, [cyclopropyl-C(═O)]₂O, CH₂═CH—C(═O)—OH, CH₃—CH═CH—C(═O)—OH, (CH₃)₂C═CH—C(═O)—OH, CH₃—CH₂—CH═CH—C(═O)—OH, CH₃—CH═C(CH₃)—C(═O)—OH, CH₂═CH—C(═O)—O—CH₃, CH₃—CH═CH—C(═O)—O—CH₃, (CH₃)₂C═CH—C(═O)—O—CH₃, CH₃—CH₂—CH═CH—C(═O)—O—CH₃, CH₃—CH═C(CH₃)—C(═O)—O—CH₃, CH₂═CH—C(═O)—O—CH₂—CH₃, CH₃—CH═CH—C(═O)—O—CH₂—CH₃, (CH₃)₂C═CH—C(═O)—O—CH₂—CH₃, CH₃—CH₂—CH═CH—C(═O)—O—CH₂—CH₃, CH₃—CH═C(CH₃)—C(═O)O—CH₂—CH₃, oxirane, 2-methyloxirane, 2-ethyloxirane, oxiran-2-yl-methanol, oxirane-2-carboxylic acid, oxirane-2-carboxylic acid methyl ester, oxirane-2-carboxylic acid ethyl ester, 2-(oxiran-2-yl)acetic acid, 2-(oxiran-2-yl)acetic acid methyl ester, 2-(oxiran-2-yl)acetic acid ethyl ester, Cl—CH₂—CH₂—OH, (CH₃—O)₂S(═O)₂, (CH₃—CH₂—O)₂S(═O)₂, (CH₃-0)₂P(═O)Cl, (CH₃—CH₂—O)₂P(═O)Cl, [(CH₃)₂CH-0]₂P(═O)Cl, NH₃, CH₃—NH₂, (CH₃)₂NH, (CH₃)₃N, CH₃—CH₂—NH₂, (CH₃—CH₂)₂NH, (CH₃—CH₂)₃N, [Cl—CH₂—CH₂—N(CH₃)₃]⁺Cl⁻, [Cl—CH₂—CH₂—N(CH₂—CH₃)₃]⁺Cl⁻, 1,3-propane sultone and 1,4-butane sultone; where the Y¹ radicals, independently of one another, preferably stand for —Cl, —Br, —I, -tosyl, -mesyl or epoxy, especially preferably for —Cl, —Br or epoxy and in particular for —Cl; and the Y² radicals, independently of one another, preferably stand for —Cl, —Br or —I, especially preferably for —Cl or —Br and in particular for —Cl.

In another preferred embodiment, at least one cyclodextrin compound, preferably only one cyclodextrin compound is mixed with only one crosslinking agent selected from the group consisting of epichlorohydrin, 1,2,3,4-diepoxybutane, 4-chloro-1,2-epoxybutane, tetramethylene diisocyanate and hexamethylene diisocyanate and preferably only one functionalizing agent, optionally in the presence of at least one solvent; where the functionalizing agent is selected from the group consisting of CH₃—Y¹, CH₃—CH₂—Y¹, cyclopropyl-Y¹, Y¹—CH₂—C(═O)—OH, Y¹—CH₂—C(═O)—O—CH₃, Y¹—CH₂—C(═O)—O—CH₂—CH₃, CH₃—C(═O)Y², dihydrofuran-2,5-dione, furan-2,5-dione, dihydro-2H-pyran-2,6(3H)-dione, [CH₃—C(═O)]₂O, [cyclopropyl-C(═O)]₂O, oxirane, 2-methyloxirane, 2-ethyloxirane, oxiran-2-yl-methanol, oxirane-2-carboxylic acid, oxirane-2-carboxylic acid methyl ester, oxirane-2-carboxylic acid ethyl ester, 2-(oxiran-2-yl)acetic acid, 2-(oxiran-2-yl)acetic acid methyl ester, 2-(oxiran-2-yl)acetic acid ethyl ester, Cl—CH₂—CH₂—OH, (CH₃—O)₂S(═O)₂, (CH₃—CH₂—O)₂S(═O)₂, CH₃—NH₂, (CH₃)₂NH, (CH₃)₃N, CH₃—CH₂—NH₂, (CH₃—CH₂)₂NH, (CH₃—CH₂)₃N, [Cl—CH₂—CH₂—N(CH₃)₃]⁺Cl⁻, [Cl—CH₂—CH₂—N(CH₂—CH₃)₃]⁺Cl⁻, 1,3-propane sultone and 1,4-butane sultone; where the Y¹ radicals, independently of one another, preferably stand for —Cl, —Br, —I, -tosyl, -mesyl or epoxy, especially preferably for —Cl, —Br or epoxy and in particular for —Cl; and Y² radicals preferably stand for —Cl, —Br or —I, especially preferably —Cl or —Br and in particular —Cl.

In another preferred embodiment, a cyclodextrin compound is mixed with the cross-linking agent epichlorohydrin and no additional crosslinking agent or with the cross-linking agent 1,2,3,4-diepoxybutane and no additional crosslinking agent, and preferably only one functionalizing agent, optionally in the presence of at least one solvent; where the functionalizing agent is selected from the group consisting of CH₃—Y¹, CH₃—CH₂—Y¹, cyclopropyl-Y¹, Y¹—CH₂—C(═O)—OH, Y¹—CH₂—C(═O)—O—CH₃, Y¹—CH₂—C(═O)—O—CH₂—CH₃, CH₃—C(═O)Y², dihydrofuran-2,5-dione, furan-2,5-dione, dihydro-2H-pyran-2,6(3H)-dione, [CH₃—C(═O)]₂O, [cyclopropyl-C(═O)]₂O, oxirane, 2-methyloxirane, 2-ethyloxirane, oxiran-2-yl-methanol, oxirane-2-carboxylic acid, oxirane-2-carboxylic acid methyl ester, oxirane-2-carboxylic acid ethyl ester, 2-(oxiran-2-yl)acetic acid, 2-(oxiran-2-yl)acetic acid methyl ester, 2-(oxiran-2-yl)acetic acid ethyl ester, Cl—CH₂—CH₂—OH, (CH₃—O)₂S(═O)₂, (CH₃—CH₂—O)₂S(═O)₂, CH₃—NH₂, (CH₃)₂NH, (CH₃)₃N, CH₃—CH₂—NH₂, (CH₃—CH₂)₂NH, (CH₃—CH₂)₃N, [Cl—CH₂—CH₂—N(CH₃)₃]⁺Cl⁻, [Cl—CH₂—CH₂—N(CH₂—CH₃)₃]⁺Cl⁻, 1,3-propane sultone and 1,4-butane sultone, where the Y¹ radicals, independently of one another, preferably stand for —Cl, —Br, —I, -tosyl, -mesyl or epoxy, especially preferably stand for —Cl, —Br or epoxy and in particular —Cl; and the Y² radicals, independently of one another, preferably stand for —Cl, —Br or —I, especially preferably —Cl or —Br and in particular —Cl.

In an especially preferred embodiment, the inventive copolymers can be obtained by mixing:

at least one, preferably only one cyclodextrin compound selected from the group consisting of (glucosyl)-cyclodextrin; (galactosyl)-cyclodextrin; (maltosyl)-cyclodextrin; (maltriosyl)-cyclodextrin; (acetyl)-cyclodextrin; (propionyl)-cyclodextrin; (isobutyryl)cyclodextrin; (butyryl)-cyclodextrin; (pivaloyl)-cyclodextrin; (pentanoyl)-cyclodextrin; (hexanoyl)-cyclodextrin; (cyclopropanecarbonyl)-cyclodextrin; (benzoyl)-cyclodextrin; (2-phenylacetyl)-cyclodextrin; (2-carboxyacetyl)-cyclodextrin; (3-carboxypropanoyl)cyclodextrin; (4-carboxybutanoyl)-cyclodextrin; (2-methoxy-2-oxoacetyl)-cyclodextrin; (3-methoxy-3-oxopropanoyl)-cyclodextrin; (4-methoxy-4-oxobutanoyl)-cyclodextrin; (2-hydroxyacetyl)-cyclodextrin; (2-hydroxypropanoyl)-cyclodextrin; (3-hydroxypropanoyl)-cyclodextrin; (2-hydroxybutanoyl)-cyclodextrin; (3-hydroxybutanoyl)-cyclodextrin;

(4-hydroxybutanoyl)-cyclodextrin; (2-hydroxypentanoyl)-cyclodextrin; (3-hydroxypentanoyl)-cyclodextrin; (4-hydroxypentanoyl)-cyclodextrin; (5-hydroxy-pentanoyl)-cyclodextrin; (2,3-dihydroxypropanoyl)-cyclodextrin; (2,3-dihydroxy-butanoyl)-cyclodextrin; (2,4-dihydroxybutanoyl)-cyclodextrin; (3,4-dihydroxybutanoyl)-cyclodextrin; (2,3-dihydroxypentanoyl)-cyclodextrin; (2,4-dihydroxypentanoyl)-cyclodextrin; (2.5-dihydroxypentanoyl)-cyclodextrin; (3,4-dihydroxypentanoyl)-cyclodextrin; (3,5-dihydroxypentanoyl)-cyclodextrin; (4,5-dihydroxypentanoyl)-cyclodextrin; (2,3,4-trihydroxybutanoyl)-cyclodextrin; (2,3,4-trihydroxypentanoyl)-cyclodextrin; (2,3,5-trihydroxypentanoyl)-cyclodextrin; (2,4,5-trihydroxypentanoyl)-cyclodextrin; (3,4,5-trihydroxypentanoyl)-cyclodextrin; (2,3,4,5-tetrahydroxy-pentanoyl)-cyclodextrin; (2-aminoacetyl)-cyclodextrin; (2-aminopropanoyl)-cyclodextrin; (2-aminobutanoyl)-cyclodextrin; [2-(1H-imidazol-1-yl)acetyl]cyclodextrin; [3-(1H-imidazol-1-yl)propanoyl]-cyclodextrin; [4-(1H-imidazol-1-yl)butanoyl]-cyclodextrin; [2-(1H-imidazol-1-yl)acetyl]-cyclodextrin; [3-(1H-imidazol-1-yl)-propanoyl]-cyclodextrin; [4-(1H-imidazol-1-yl)butanoyl]-cyclodextrin; [2-(pyridin-3-yl)-acetyl]cyclodextrin; [3-(pyridin-3-yl)propanoyl]-cyclodextrin; [4-(pyridin-3-yl)butanoyl]-cyclodextrin; (methyl)-cyclodextrin; (ethyl)cyclodextrin; (isopropyl)-cyclodextrin; (N-propyl)-cyclodextrin; (isobutyl)-cyclodextrin; (N-butyl)-cyclodextrin; (sec-butyl)-cyclodextrin; (tert-butyl)-cyclodextrin; (cyclopropyl)cyclodextrin; (phenyl)-cyclodextrin; (benzyl)-cyclodextrin; (phenethyl)-cyclodextrin; (carboxymethyl)-cyclodextrin; (1-carboxyethyl)-cyclodextrin; (2-carboxyethyl)-cyclodextrin; (1-carboxypropyl)-cyclodextrin; (2-carboxypropyl)-cyclodextrin; (3-carboxypropyl)-cyclodextrin; (1-carboxypropan-2-yl)-cyclodextrin; (4-carboxybutyl)-cyclodextrin; (5-carboxypentyl)-cyclodextrin; (5-carboxypentyl)-cyclodextrin; (2-methoxy-2-oxoethyl)-cyclodextrin; (3-methoxy-2-oxopropyl)-cyclodextrin; (4-methoxy-4-oxobutyl)-cyclodextrin; (5-methoxy-5-oxopentyl)-cyclodextrin; (6-methoxy-6-oxohexyl)cyclodextrin; (2-ethoxy-2-oxoethyl)-cyclodextrin; (3-ethoxy-2-oxopropyl)-cyclodextrin; (4-ethoxy-4-oxobutyl)-cyclodextrin; (5-ethoxy-5-oxopentyl)-cyclodextrin; (6-ethoxy-6-oxohexyl)-cyclodextrin; (3-carboxy-2-hydroxypropyl)-cyclodextrin; (2-hydroxy-4-methoxy-4-oxobutyl)-cyclodextrin; (4-ethoxy-2-hydroxy-4-oxobutyl)-cyclodextrin; (1-carboxy-2-hydroxyethyl)-cyclodextrin; (3-hydroxy-1-methoxy-1-oxopropan-2-yl)-cyclodextrin; (1-ethoxy-3-hydroxy-1-oxopropan-2-yl)-cyclodextrin; (1-carboxy-3-hydroxypropan-2-yl)-cyclodextrin; (1-hydroxy-4-methoxy-4-oxobutan-2-yl)-cyclodextrin; (4-ethoxy-1-hydroxy-4-oxobutan-2-yl)-cyclodextrin; (hydroxymethyl)-cyclodextrin; (1-hydroxyethyl)-cyclodextrin; (2-hydroxyethyl)-cyclodextrin; (1-hydroxypropyl)-cyclodextrin; (2-hydroxypropyl)-cyclodextrin; (1-hydroxypropan-2-yl)-cyclodextrin; (2-hydroxypropan-2-yl)-cyclodextrin; (1-hydroxybutyl)-cyclodextrin; (2-hydroxybutyl)cyclodextrin; (3-hydroxybutyl)-cyclodextrin, (4-hydroxybutyl)-cyclodextrin; (1-hydroxypentyl)-cyclodextrin; (2-hydroxypentyl)-cyclodextrin; (3-hydroxypentyl)-cyclodextrin; (4-hydroxypentyl)-cyclodextrin; (5-hydroxypentyl)-cyclodextrin; (1,2-dihydroxyethyl)cyclodextrin, (1,2-dihydroxypropyl)-cyclodextrin; (1,3-dihydroxypropyl)-cyclodextrin; (2,3-dihydroxypropyl)-cyclodextrin; (1,2-dihydroxybutyl)-cyclodextrin; (1,3-dihydroxybutyl)-cyclodextrin; (1,4-dihydroxybutyl)-cyclodextrin; (2,3-dihydroxybutyl)-cyclodextrin; (2,4-dihydroxybutyl)-cyclodextrin; (3,4-dihydroxybutyl)-cyclodextrin; (1,2-dihydroxypentyl)-cyclodextrin; (1,3-dihydroxypentyl)-cyclodextrin; (1,4-dihydroxypentyl)-cyclodextrin; (1,5-dihydroxypentyl)-cyclodextrin, (2,3-dihydroxypentyl)-cyclodextrin; (2,4-dihydroxypentyl)-cyclodextrin; (2.5-dihydroxypentyl)-cyclodextrin; (3,4-dihydroxypentyl)-cyclodextrin; (3,5-dihydroxypentyl)-cyclodextrin; (4,5-dihydroxypentyl)-cyclodextrin; (1,2,3-trihydroxypropyl)-cyclodextrin; (1,2,3-trihydroxybutyl)cyclodextrin; (1,2,4-trihydroxybutyl)-cyclodextrin; (1,3,4-trihydroxybutyl)-cyclodextrin; (2,3,4-trihydroxybutyl)-cyclodextrin; (1,2,3-trihydroxypentyl)-cyclodextrin; (1,2,4-trihydroxypentyl)-cyclodextrin; (1,2,5-trihydroxypentyl)-cyclodextrin; (1,3,4-trihydroxypentyl)-cyclodextrin; (1,3,5-trihydroxypentyl)-cyclodextrin, (1,4,5-trihydroxypentyl)cyclodextrin; (2,3,4-trihydroxypentyl)-cyclodextrin; (2,3,5-trihydroxypentyl)-cyclodextrin; (2,4,5-trihydroxypentyl)-cyclodextrin; (3,4,5-trihydroxypentyl)-cyclodextrin; (1,2,3,4-tetrahydroxybutyl)-cyclodextrin; (1,2,3,4-tetrahydroxypentyl)-cyclodextrin; (1,2,4,5-tetrahydroxypentyl)-cyclodextrin; (2,3,4,5-tetrahydroxypentyl)-cyclodextrin; (1,2,3,4,5-pentahydroxypentyl)-cyclodextrin; [(sulfoxy)methyl]-cyclodextrin; [2-(sulfoxy)ethyl]-cyclodextrin; [3-(sulfoxy)propyl]-cyclodextrin; [4-(sulfoxy)butyl]-cyclodextrin; [(phosphonooxy)methyl]-cyclodextrin; [2-(phosphonooxy)ethyl]-cyclodextrin; [3-(phosphonooxy)propyl]-cyclodextrin; [4-(phosphonooxy)butyl]-cyclodextrin; [4-(phosphonooxy)butyl]-cyclodextrin; (sulfomethyl)-cyclodextrin; (2-sulfoethyl)-cyclodextrin; (3-sulfopropyl)-cyclodextrin; (4-sulfobutyl)-cyclodextrin; (5-sulfopentyl)-cyclodextrin; (6-sulfohexyl)-cyclodextrin; (phosphonomethyl)-cyclodextrin; (2-phosphonoethyl)-cyclodextrin; (3-phosphonopropyl)-cyclodextrin; (4-phosphonobutyl)-cyclodextrin; (5-phosphonopentyl)-cyclodextrin; (6-phosphonohexyl)-cyclodextrin; (2-phosphonovinyl)cyclodextrin; (3-phosphonoallyl)-cyclodextrin; (4-phosphonobut-3-enyl)-cyclodextrin; (5-phosphonopent-4-enyl)-cyclodextrin; (6-phosphonohex-5-enyl)-cyclodextrin; (aminomethyl)-cyclodextrin; (2-aminoethyl)-cyclodextrin; (3-aminopropyl)-cyclodextrin; (4-aminobutyl)-cyclodextrin; (5-aminopentyl)-cyclodextrin; (6-aminohexyl)cyclodextrin, [(N,N-dimethylamino)methyl]-cyclodextrin; [2-(N,N-dimethylamino)ethyl]-cyclodextrin; [3-(N,N-dimethylamino) propyl]-cyclodextrin; [4-(N,N-dimethylamino)butyl]-cyclodextrin; [5-(N,N-dimethylamino)pentyl]-cyclodextrin; [6-(N,N-dimethylamino) hexyl]-cyclodextrin; [(N,N-diethylamino)methyl]-cyclodextrin; [2-(N,N-diethylamino)ethyl]-cyclodextrin; [3-(N,N-diethylamino)propyl]-cyclodextrin; [4-(N,N-diethylamino)butyl]-cyclodextrin; [5-(N,N-diethylamino)pentyl]-cyclodextrin; [6-(N,N-diethylamino)hexyl]-cyclodextrin; [(trimethylammonio)methyl]-cyclodextrin chloride; [2-(trimethylammonio)ethyl]-cyclodextrin chloride; [3-(trimethylammonio)propyl]-cyclodextrin chloride; [4-(trimethylammonio)butyl]-cyclodextrin chloride; [5-(trimethylammonio)-pentyl]-cyclodextrin chloride; [6-(trimethylammonio)hexyl]-cyclodextrin chloride; [(triethylammonio)methyl]-cyclodextrin chloride; [2-(triethylammonio)ethyl]-cyclodextrin chloride; [3-(triethylammonio)propyl]-cyclodextrin chloride; [4-(triethylammonio)butyl]-cyclodextrin chloride; [5-(triethylammonio)pentyl]-cyclodextrin chloride; [6-(triethylammonio)hexyl]-cyclodextrin chloride; [(1H-imidazol-1-yl)methyl]-cyclodextrin; [2-(1H-imidazol-1-yl)ethyl]-cyclodextrin; [3-(1H-imidazol-1-yl)propyl]-cyclodextrin; [4-(1H-imidazol-1-yl)butyl]-cyclodextrin; (pyridin-3-ylmethyl)-cyclodextrin; [2-(pyridin-3-yl)ethyl]-cyclodextrin; [3-(pyridin-3-yl)propyl]-cyclodextrin; [4-(pyridin-3-yl)butyl]-cyclodextrin; [(β-D-glucopyranosyloxyuronic acid) methyl]-cyclodextrin; [2-(β-D-glucopyranosyloxyuronic acid) ethyl]-cyclodextrin; [3-(β-D-glucopyranosyloxyuronic acid)propyl]-cyclodextrin; [4-(β-D-glucopyranosyloxyuronic acid) butyl]-cyclodextrin; [5-(β-D-glucopyranosyloxyuronic acid)pentyl]-cyclodextrin; and [6-(β-D-gluco pyranosyloxyuronic acid) hexyl]-cyclodextrin; where the term “cyclodextrin” stands for α-, β- or γ-cyclodextrin, preferably for α- or β-cyclodextrin and in particular for β-cyclodextrin;

with at least one, preferably only one crosslinking agent selected from the group consisting of epichlorohydrin, 4-chloro-1,2-epoxybutane, 1,2,3,4-diepoxybutane, tetramethylene diisocyanate and hexamethylene diisocyanate; where the crosslinking agent epichlorohydrin in preferred in particular; and
with at least one, preferably only one, functionalizing agent, selected from the group consisting of CH₃—Y¹, CH₃—CH₂—Y¹, cyclopropyl-Y¹, Y¹—CH₂—C(═O)—OH, Y¹—CH₂—C(═O)—O—CH₃, Y¹—CH₂—C(═O)—O—CH₂—CH₃, CH₃—C(═O)Y², dihydrofuran-2,5-dione, furan-2,5-dione, dihydro-2H-pyran-2,6(3H)-dione, [CH₃—C(═O)]₂O, [cyclopropyl-C(═O)]₂O, oxirane, 2-methyloxirane, 2-ethyloxirane, oxiran-2-yl-methanol, oxirane-2-carboxylic acid, oxirane-2-carboxylic acid methyl ester, oxirane-2-carboxylic acid ethyl ester, 2-(oxiran-2-yl)acetic acid, 2-(oxiran-2-yl)acetic acid methyl ester, 2-(oxiran-2-yl)acetic acid ethyl ester, Cl—CH₂—CH₂—OH, (CH₃—O)₂S(═O)₂, (CH₃—CH₂—O)₂S(═O)₂, CH₃—NH₂, (CH₃)₂NH, (CH₃)₃N, CH₃—CH₂—NH₂, (CH₃—CH₂)₂NH, (CH₃—CH₂)₃N, [Cl—CH₂—CH₂—N(CH₃)₃]⁺Cl⁻, [Cl—CH₂—CH₂—N(CH₂—CH₃)₃]⁺Cl⁻, 1,3-propane sultone and 1,4-butane sultone; where the Y¹ radicals, independently of one another, preferably stand for —Cl, —Br, —I, -tosyl, -mesyl or epoxy, especially preferably —Cl, —Br or epoxy and in particular for —Cl, and Y² preferably stands for —Cl, —Br or —I, especially preferably stands for —Cl or —Br and in particular for —Cl;
optionally in the presence of at least one solvent.

In another especially preferred embodiment, unsubstituted α-. β- or γ-cyclodextrin is reacted with the crosslinking agent epichlorohydrin and the functionalizing agent 1,3-butane sultone, 1,4-butane sultone, 2-chloroacetic acid, 2-chloroacetic acid methyl ester or 2-chloroacetic acid ethyl ester, optionally in the presence of a solvent.

The mixing of the cyclodextrin compounds with crosslinking agents and functionalizing agents may be performed in aqueous or organic solvents. They are preferably mixed in water, dimethyl sulfoxide (DMSO), dimethylformamide (DMF), N-methyl-2-pyrrolidone (NMP) or mixtures thereof. The reaction may also take place in anhydrous DMSO, anhydrous DMF, anhydrous NMP or mixtures thereof, preferably under anhydrous conditions. Especially advantageous solvents are water, DMSO and DMF as well as mixtures thereof, in particular water or DMF and mixtures thereof.

The mixing is preferably performed in an alkaline medium, usually at a pH of >7.

The mixing is preferably performed at pH 7.5≦pH≦14, more preferably at 7.5≦pH≦13, even more preferably at 8≦pH≦13, most preferably at 8≦pH≦12.5 and in particular at 9≦pH≦12.

The pH of the reaction solution or dispersion is preferably adjusted with bases, for example, basic alkali metal salts, basic alkaline earth metal salts, hydrides, alkoxides or organic bases, for example, pyridine or diisopropylamine. Sodium hydroxide, sodium bicarbonate, sodium carbonate, potassium hydroxide, potassium bicarbonate, potassium carbonate, sodium hydride, potassium hydride, sodium methanolate, potassium tert-butoxide, pyridine and diisopropylamine are especially advantageous.

Since copolymerization and/or crosslinking of the cyclodextrin compounds are performed under basic reaction conditions, substituents which are not stable in an alkaline medium may be structurally altered. This change may affect base-labile groups in cyclodextrin compounds, crosslinking agents and functionalizing agents. For example, the ester group of the cyclodextrin compound (2-methoxy-2-oxoethyl)cyclodextrin may be cleaved to form a carboxyl group or a carboxylate group, such that the starting compound (2-methoxy-2-oxoethyl)-cyclodextrin is converted to (carboxymethyl)-cyclodextrin. This conversion may be complete or incomplete, based on the total number of ester groups. Consequently, some or all of the ester groups in the corresponding inventive copolymer may be converted to carboxyl groups.

In a preferred embodiment, the cyclodextrin compounds are dissolved or dispersed in a solvent or solvent mixture, and then the crosslinking agents and the functionalizing agents are dissolved or dispersed therein, preferably while stirring the dispersion or solution. The crosslinking agents and the functionalizing agents may be added simultaneously or in any order, all at once or successively in portions. The cross-linking agents and/or functionalizing agents may be dissolved or dispersed in a solvent (e.g., water, DMF, DMSO, NMP) before being added and then added to the reaction mixture as a solution or dispersion all at once or successively in portions or added by drops by means of a dropping funnel, a pipette or a syringe. The addition and/or dropwise addition may be performed with the help of a syringe pump.

In another preferred embodiment, the crosslinking agents and functionalizing agents are dissolved or dispersed in any order or simultaneously in a solvent or solvent mixture, and then the cyclodextrin compounds are dissolved or dispersed in this solution or dispersion, preferably while stirring the solution or dispersion. The cyclodextrin compounds may be added all at once or successively in portions. Before being added, the cyclodextrin compounds may be dissolved or dispersed in the solvent and then added to the reaction mixture as a solution or dispersion all at once or successively in portions or added by drops by using a dropping funnel, pipette or syringe. The addition and/or dropwise addition may be performed with the help of a syringe pump.

In another preferred embodiment, the crosslinking agents, functionalizing agents and cyclodextrin compounds are dissolved or dispersed simultaneously in the solvent or solvent mixture.

In an especially preferred embodiment, substituted or unsubstituted cyclodextrin compounds are first mixed with crosslinking agents in the presence of a solvent and then are reacted to form crosslinked copolymers. The resulting crosslinked copolymers may be purified and isolated after crosslinking. Next the functionalizing agent may be mixed and reacted with the purified or isolated copolymer in the presence of a solvent; or the functionalizing agent is mixed and reacted with the reaction mixture containing the unisolated and/or unpurified copolymers.

In an especially preferred embodiment, substituted or unsubstituted cyclodextrin compounds are first mixed with functionalizing agents in the presence of a solvent and are reacted to form substituted cyclodextrin compounds. The resulting substituted cyclodextrin compounds may be purified and isolated after crosslinking. Next the crosslinking agent may be mixed and reacted with the purified or isolated substituted cyclodextrin compounds in the presence of a solvent; or the crosslinking agent is mixed and reacted with the reaction mixture containing the unisolated and/or unpurified substituted cyclodextrin compounds.

There may be a sudden and unwanted increase in reaction temperature during mixing. It is therefore advantageous to optionally cool the reaction mixture to ensure an essentially constant reaction temperature. The reaction mixture may be cooled in an ice bath or a water bath.

The reaction mixture may be alkalized during or after addition of the crosslinking agent, before, during or after addition of the cyclodextrin compounds, before, during or after addition of the functionalizing agents or before, during or after addition of a mixture thereof.

The alkaline pH is preferably measured by using pH paper or a pH meter. In a preferred embodiment, the pH is measured continuously during the reaction or at certain intervals. If the pH drops below a certain level (e.g., pH<8 or <7.5), an alkaline pH in the reaction mixture can be restored by adding a base.

The reaction preferably takes place at a reaction temperature (temperature of the reaction mixture) of 5.0 to 90° C., more preferably 10 to 75° C., even more preferably 15 to 50° C., most preferably 15 to 30° C. and in particular 20 to 25° C. After mixing the cyclodextrin compounds with the crosslinking agents and functionalizing agents, optionally in the presence of a solvent or solvent mixture, the temperature of the reaction mixture is preferably adjusted to 5 to 90° C., more preferably 10 to 75° C., even more preferably 15 to 50° C., most preferably 15 to 30° C. and in particular 20 to 25° C.

In a preferred embodiment, a total substance quantity of ≧0.5 mol, ≧1.0 mol, mol or ≧3.0 mol, more preferably ≧4.0 mol, mol or ≧6.0 mol, even more preferably ≧7.0 mol, mol or ≧9.0 mol, most preferably mol, ≧15 mol or mol, and in particular ≧25 mol of crosslinking agent, based on a total substance quantity of 1.0 mol of cyclodextrin compounds, is used in the reaction mixture (equivalents).

In another preferred embodiment, a total substance quantity of ≧0.5 mol, ≧1.0 mol, ≧2.0 mol or ≧3.0 mol, more preferably ≧4.0 mol, ≧5.0 mol or ≧6.0 mol, even more preferably ≧7.0 mol, mol or ≧9.0 mol, most preferably ≧10 mol, ≧15 mol or ≧20 mol, and in particular ≧25 mol of functionalizing agent, based on a total substance quantity of 1.0 mol of cyclodextrin compounds, is used in the reaction mixture (equivalents).

In another preferred embodiment, the ratio of the total substance quantity of cross-linking agent to the total substance quantity of cyclodextrin compounds is ≧1:1, more preferably ≧2:1, even more preferably ≧3:1, most preferably ≧5:1 and in particular ≧10:1.

In another preferred embodiment, the ratio of the total substance quantity of functionalizing agents to the total substance quantity of cyclodextrin compounds is ≧1:1, more preferably ≧2:1, even more preferably ≧3:1, most preferably ≧5:1 and in particular ≧10:1.

In another preferred embodiment, the ratio of the total substance quantity of functionalizing agents to the total substance quantity of crosslinking agents is ≧1:1, more preferably ≧2:1, even more preferably ≧3:1, most preferably ≧5:1 and in particular ≧10:1.

The reaction (crosslinking reaction) can be followed by means of suitable analytical processes. For example, random samples of the reaction mixture may be taken at certain points in time and analyzed by LC/MS or NMR. The reaction product present in the reaction mixture at a certain point in time can be determined in this way. If the random sample analysis reveals that the desired reaction product has been obtained, then the reaction can be terminated, and the desired reaction product can be isolated or purified.

To terminate the reaction, the reaction mixture is preferably neutralized, i.e., the pH of the reaction solution or dispersion is preferably adjusted to 6.5≦pH≦7.5, especially preferably to 6.8≦pH≦7.2. The mixture is preferably neutralized using organic acids (e.g., trifluoroacetic acid, acetic acid) or inorganic acids (e.g., hydrochloric acid, sulfuric acid, nitric acid).

In another preferred embodiment, after the reaction and/or neutralization, the reaction mixture is dialyzed, preferably dialyzed in a salt-free process, in which the dialysis membrane used in this dialysis process preferably has an MWCO (“molecular weight cut-off”) of 0.50 kDa or 1.0 kDa, more preferably of 2.0 kDa or 3.0 kDa, even more preferably of 4.0 kDa or 5.0 kDa, most preferably of 10 kDa and in particular of ≦15 kDa.

The salt-free dialyzed inventive copolymer preferably has a residual content of compounds with a molecular weight of <1.5 kDa, based on the total weight of the inventive copolymer, of ≦10 wt %, more preferably ≦5.0 wt %, even more preferably ≦2.5 wt % and in particular ≦1.0 wt %.

The reaction mixture and/or the essentially salt-free dialysis solution may be concentrated preferably with the help of a rotary evaporator before further purification and/or isolation. The reaction mixture can also be concentrated even before dialysis, preferably salt-free dialysis.

The inventive copolymers are isolated or purified preferably by means of ultrafiltration, column chromatography (e.g., on silica gel or activated carbon), preparative HPLC or recrystallization. The resulting inventive copolymer can be lyophilized.

In a preferred embodiment, the substance quantity proportion of the linkers contained in the inventive copolymer is 20 to 95 mol %, more preferably 25 to 80 mol %, even more preferably 30 to 70 mol %, most preferably 30 to 60 mol % and in particular 30 to 50 mol %, based on the total substance quantity of the monomers contained in the inventive copolymer.

The substance quantity proportion of the cyclodextrin monomers contained in the inventive copolymer preferably amounts to 5.0 to 80 mol %, more preferably 10 to 75 mol %, even more preferably 20 to 70 mol %, most preferably 30 to 70 mol % and in particular 40 to 70 mol %, based on the total substance quantity of the monomers contained in the inventive copolymer.

In another preferred embodiment, the sum of the substance quantity proportion of the linkers and the substance quantity proportion of the cyclodextrin monomers is ≧25 mol %, ≧30 mol % or ≧40 mol %, more preferably ≧50 mol % or ≧60 mol %, even more preferably ≧70 mol % or ≧80 mol %, most preferably ≧90 mol % or ≧95 mol %, and in particular 100 mol %, based on the total substance quantity of the monomers contained in the inventive copolymer.

The water solubility of the inventive copolymers at 23° C. is preferably ≧5.0 g l⁻¹, ≧10 g l⁻¹, ≧25 g l⁻¹ or ≧50 g l⁻¹, more preferably ≧75 g l⁻¹, ≧100 g l⁻¹, ≧125 g l⁻¹ or ≧150 g l⁻¹, even more preferably ≧175 g l⁻¹, ≧200 g l⁻¹, ≧225 g l⁻¹ or ≧250 g l⁻¹, most preferably ≧300 g l⁻¹ or ≧400 g l⁻¹ and in particular ≧500 g l⁻¹.

The osmolality of a 50 mM aqueous solution of inventive copolymer is preferably ≧250 mosm kg⁻¹, ≧75 mosm kg⁻¹ or ≧100 mosm kg⁻¹, more preferably ≧125 mosm kg⁻¹, ≧150 mosm kg⁻¹ or ≧175 mosm kg⁻¹, even more preferably ≧200 mosm kg⁻¹ or ≧225 mosm kg⁻¹, most preferably ≧250 mosm kg⁻¹ or ≧275 mosm kg⁻¹ and in particular ≧300 mosm kg⁻¹.

The degree of polymerization of the inventive copolymers is preferably ≧2.0 or ≧3.0, more preferably ≧4.0 or ≧5.0, even more preferably ≧6.0 or ≧7.0, most preferably ≧8.0 or ≧9.0 and in particular ≧10.

The average molecular weight of the inventive copolymers is preferably ≧2.0 kDa, more preferably ≧2.5 kDa, even more preferably ≧3.0 kDa, most preferably ≧4.0 kDa and in particular ≧5.0 kDa. The average molecular weight is preferably measured with the help of a membrane osmometer (distilled water, 23° C.).

In another preferred embodiment, the average molecular weight (M) of the inventive polymers is preferably 2.0≦M≦100 kDa, more preferably 2.0≦M≦50 kDa, even more preferably 2.0≦M≦25 kDa, most preferably 2.0≦M≦15 kDa and in particular 2.0≦M≦10 kDa.

In another especially preferred embodiment, the inventive copolymer that can be obtained by mixing at least one cyclodextrin compound with at least one crosslinking agent and at least one functionalizing agent preferably has a degree of polymerization of ≧2.0 or ≧3.0, more preferably ≧4.0 or ≧5.0, even more preferably ≧6.0 or ≧7.0, most preferably ≧8.0 or ≧9.0 and in particular ≧10.

In another preferred embodiment, the average particle diameter of the inventive copolymers that can be obtained by mixing at least one cyclodextrin compound with at least one crosslinking agent and at least one functionalizing agent is ≦1000 μm, ≦900 μm or ≦800 μm, more preferably ≦700 μm, ≦600 μm or ≦500 μm, even more preferably ≦400 μm, ≦300 μm or ≦200 μm, most preferably ≦100 μm, ≦75 μm or ≦50 μm and in particular ≦25 μm.

Another preferred subject matter of this invention is copolymers that can be obtained by a process comprising the process step:
mixing at least one cyclodextrin polymer with at least one functionalizing agent;
for use in the treatment of diseases of the urogenital system.

For the purposes of this invention, the term “cyclodextrin polymer” comprises unsubstituted copolymers or mono- or polysubstituted copolymers as defined above, i.e., containing at least two cyclodextrin monomers and at least one linker; such that the substituted cyclodextrin polymers contain at least one unsubstituted —OH, at least one unsubstituted —SH or at least one primary, secondary or tertiary amino group.

In the sense of this invention, “the at least one unsubstituted —OH” may be part of the glycosyl units of the cyclodextrin monomer, the linkers, the substituents of the cyclodextrin monomers and/or the substituents of the linkers. For the purposes of this invention, “the at least one —SH” or “the at least one primary, secondary or tertiary amino group” are preferably components of the substituents of the cyclodextrin monomers or of the substituents of the linkers. The cyclodextrin monomers and linkers contained in the cyclodextrin polymers as well as their preferred embodiments are defined as indicated above.

Furthermore, for the purposes of this invention, the term “cyclodextrin polymer” comprises copolymers that can be obtained by the process defined above, i.e., by the process comprising the process step:
mixing at least one cyclodextrin compound with
   (a) at least one crosslinking agent or
   (b) at least one crosslinking agent and at least one
   functionalizing agent;
      including the preferred embodiments of these copolymers as defined above; wherein the substituted cyclodextrin polymers contain at least one unsubstituted OH, at least one unsubstituted —SH or at least one primary, secondary or tertiary amino group.

In a preferred embodiment, the cyclodextrin polymer contains two or more unsubstituted hydroxyl groups, two or more unsubstituted thiol groups or two or more primary, secondary or tertiary amino groups.

In another preferred embodiment, substituted α-cyclodextrin polymers contain on the average 1, 2, 3, 4, 5, 6, 7, 8, 9, 10, 11, 12, 13, 14, 15, 16 or 17 unsubstituted hydroxyl groups, based on an α-cyclodextrin monomer of the α-cyclodextrin polymer.

In another preferred embodiment, substituted β-cyclodextrin polymers contain on the average 1, 2, 3, 4, 5, 6, 7, 8, 9, 10, 11, 12, 13, 14, 15, 16, 17, 18, 19 or 20 unsubstituted hydroxyl groups, based on a β-cyclodextrin monomer of the β-cyclodextrin polymer.

In another preferred embodiment, substituted β-cyclodextrin polymers contain on the average 1, 2, 3, 4, 5, 6, 7, 8, 9, 10, 11, 12, 13, 14, 15, 16, 17, 18, 19, 20, 21, 22 or 23 unsubstituted hydroxyl groups, based on a γ-cyclodextrin monomer of the γ-cyclodextrin polymer.

In another preferred embodiment, substituted α/β-cyclodextrin polymers contain on the average 1, 2, 3, 4, 5, 6, 7, 8, 9, 10, 11, 12, 13, 14, 15, 16, 17, 18, 19 or 20 unsubstituted hydroxyl groups, based on a cyclodextrin monomer of the α/β-cyclodextrin polymer.

In another preferred embodiment, substituted α/γ-cyclodextrin polymers contain on the average 1, 2, 3, 4, 5, 6, 7, 8, 9, 10, 11, 12, 13, 14, 15, 16, 17, 18, 19, 20, 21, 22 or 23 unsubstituted hydroxyl groups, based on a cyclodextrin monomer of the α/γ-cyclodextrin polymer.

In another preferred embodiment, substituted β/γ-cyclodextrin polymers contain on the average 1, 2, 3, 4, 5, 6, 7, 8, 9, 10, 11, 12, 13, 14, 15, 16, 17, 18, 19, 20, 21, 22 or 23 unsubstituted hydroxyl groups, based on a cyclodextrin monomer of the β/γ-cyclodextrin polymer.

In another preferred embodiment, substituted α/β/γ-cyclodextrin polymers contain on the average 1, 2, 3, 4, 5, 6, 7, 8, 9, 10, 11, 12, 13, 14, 15, 16, 17, 18, 19, 20, 21, 22 or 23 unsubstituted hydroxyl groups, based on a cyclodextrin monomer of the α/β/γ-cyclodextrin polymer.

The functionalizing agents used in the process, including the preferred embodiments, are as defined above.

The inventive copolymers can thus be obtained by a process comprising the process step:
mixing at least one already copolymerized and/or crosslinked unsubstituted cyclodextrin polymer or a mono- or polysubstituted cyclodextrin polymer with at least one functionalizing agent.

In a preferred embodiment, the inventive copolymers can be obtained by a process comprising the process step:
mixing at least one already polymerized and/or crosslinked unsubstituted cyclodextrin polymer with at least one functionalizing agent.

The term “cyclodextrin polymer” preferably comprises α-cyclodextrin polymers, β-cyclodextrin polymers, γ-cyclodextrin polymers, α/β-cyclodextrin polymers, α/γ-cyclodextrin polymers and α/β/γ-cyclodextrin polymers as well as mixtures containing at least two different cyclodextrin polymers of those mentioned above; such that the cyclodextrin polymers are unsubstituted or are mono- or polysubstituted polymers, preferably being unsubstituted, and having at least one unsubstituted —OH, at least one unsubstituted —SH or at least one primary, secondary or tertiary amino group.

In the sense of this invention, the term “α-cyclodextrin polymer” stands for copolymers containing two or more α-cyclodextrin monomers, which may be the same or different, and one or more linkers, which may be the same or different; the term “β-cyclodextrin polymer” stands for copolymers containing two or more β-cyclodextrin monomers that may be the same or different and one or more linkers that may be the same or different; the term “γ-cyclodextrin polymer” stands for copolymers containing two or more γ-cyclodextrin monomers that may be the same or different and one or more linkers that may be the same or different; the term “α/β-cyclodextrin polymers” stands for copolymers containing one or more α-cyclodextrin monomers that may be the same or different, one or more β-cyclodextrin monomers that may be the same or different and one or more linkers that may be the same or different; the term “α/γ-cyclodextrin polymer” stands for copolymers containing one or more α-cyclodextrin monomers that may be the same or different, one or more γ-cyclodextrin monomers that may be the same or different and one or more linkers that may be the same or different; the term “β/γ-cyclodextrin polymer” stands for copolymers containing one or more β-cyclodextrin monomers that may be the same or different, one or more γ-cyclodextrin monomers that may be the same or different and one or more linkers that may be the same or different; the term “α/β/γ-cyclodextrin polymer” stands for copolymers containing one or more α-cyclodextrin monomers that may be the same or different, one or more β-cyclodextrin monomers that may be the same or different, one or more γ-cyclodextrin monomers that may be the same or different and two or more linkers that may be the same or different; such that the cyclodextrin polymers are unsubstituted or mono- or polysubstituted, preferably being unsubstituted, and having at least one unsubstituted —OH, at least one unsubstituted —SH or at least one primary, secondary or tertiary amino group.

In another preferred embodiment, the term “cyclodextrin polymer” stands for
α-, β- and γ-cyclodextrin-epichlorohydrin copolymer,
α-, β- and γ-cyclodextrin-4-chloro-1,2-epoxybutane copolymer,
α-, β- and γ-cyclodextrin-1,2,3,4-diepoxybutane copolymer,
α-, β- and γ-cyclodextrin-tetramethylene diisocyanate copolymer or
α-, β- and γ-cyclodextrin-hexamethylene diisocyanate copolymer;
   i.e., the unsubstituted cyclodextrin polymers or the mono- or polysubstituted cyclodextrin polymers are preferably copolymerized and/or crosslinked with epichlorohydrin, 4-chloro-1,2-epoxybutane, 1,2,3,4-diepoxybutane, tetramethylene diisocyanate or hexamethylene diisocyanate.

Unsubstituted α-, β- and γ-cyclodextrin-epichlorohydrin copolymers or those substituted one or more times are especially advantageous.

In a preferred embodiment, the substance quantity proportion of the linkers contained in the cyclodextrin polymer is 20 to 95 mol %, more preferably 25 to 80 mol %, even more preferably 30 to 70 mol %, most preferably 30 to 60 mol % and in particular 30 to 50 mol %, based on the total substance quantity of the monomers contained in the cyclodextrin polymer.

The substance quantity proportion of the cyclodextrin monomers contained in the cyclodextrin polymer is preferably 5.0 to 80 mol %, more preferably 10 to 75 mol %, even more preferably 20 to 70 mol %, most preferably 30 to 70 mol % and in particular 40 to 70 mol %, based on the total substance quantity of the monomers contained in the cyclodextrin polymer.

In another preferred embodiment, the sum of the substance quantity proportion of the linkers and the substance quantity proportion of the cyclodextrin monomers is ≧25 mol %, ≧30 mol % or ≧40 mol %, more preferably ≧50 mol % or ≧60 mol %, even more preferably ≧70 mol % or ≧80 mol %, most preferably ≧90 mol % or ≧95 mol %, and in particular 100 mol %, based on the total substance quantity of the monomers contained in the cyclodextrin polymer.

The cyclodextrin polymers may have a low water solubility. The water solubility of the cyclodextrin polymers can be increased by mixing them with functionalizing agent.

The water solubility of the cyclodextrin polymers at 23° C. is preferably ≦1.0 g l⁻¹, ≦2.5 g l⁻¹, ≦5.0 g l⁻¹ or ≦7.5 g l⁻¹, more preferably ≦10 g l⁻¹, ≦20 g l⁻¹, ≦40 g l⁻¹ or ≦60 g l⁻¹, even more preferably ≦80 g l⁻¹, ≦100 g l⁻¹, ≦125 g l⁻¹ or ≦150 g l⁻¹, most preferably ≦175 g l⁻¹ or ≦200 g l⁻¹ and in particular ≦225 g l⁻¹.

The cyclodextrin polymers may have a low osmolality. The osmolality of the cyclodextrin polymers may be increased by mixing with functionalizing agent.

The osmolality of a 50 mM aqueous solution cyclodextrin polymer is preferably ≦10 mosm kg⁻¹, ≦25 mosm kg⁻¹ or ≦50 mosm kg⁻¹, more preferably ≦75 mosm kg⁻¹, ≦100 mosm kg⁻¹ or ≦125 mosm kg⁻¹, even more preferably ≦150 mosm kg⁻¹ or ≦175 mosm kg⁻¹, most preferably ≦200 mosm kg⁻¹ or ≦225 mosm kg⁻¹ and in particular ≧300 mosm kg⁻¹.

The degree of polymerization of the cyclodextrin polymers is preferably ≧2.0 or ≧3.0, more preferably ≧4.0 or ≧5.0, even more preferably ≧6.0 or ≧7.0, most preferably ≧8.0 or ≧9.0 and in particular ≧10.

The average particle diameter of the inventive copolymers is preferably ≦1000 μm, ≦900 μm or ≦800 μm, more preferably ≦700 μm, ≦600 μm or ≦50 μm, even more preferably ≦400 μm, ≦300 μm or ≦200 μm, most preferably ≦100 μm, ≦75 μm or ≦50 μm and in particular ≦25 μm.

The average molecular weight of the cyclodextrin polymers is preferably ≧2.0 kDa, more preferably ≧2.5 kDa, even more preferably ≧3.0 kDa, most preferably ≧4.0 kDa and in particular ≧5.0 kDa. The average molecular weight is preferably measured with the help of a membrane osmometer (distilled water, 23° C.).

In another preferred embodiment, the average molecular weight (M) of the cyclodextrin polymers is preferably 2.0≦M≦100 kDa, more preferably 2.0≦M≦50 kDa, even more preferably 2.0≦M≦25 kDa, most preferably 2.0≦M≦15 kDa and in particular 2.0≦M≦10 kDa.

In another especially preferred embodiment, the cyclodextrin polymer preferably has a degree of polymerization of ≧2.0 or ≧3.0, more preferably ≧4.0 or ≧5.0, even more preferably ≧6.0 or ≧7.0 most preferably ≧8.0 or ≧9.0 and in particular ≧10.

The inventive copolymers can preferably be obtained by a process comprising the process step of mixing α-cyclodextrin polymer with one or more functionalizing agents (e.g., 2, 3 or 4), preferably with only one functionalizing agent; mixing β-cyclodextrin polymer with one or more functionalizing agents (e.g., 2, 3 or 4), preferably with only one functionalizing agent; mixing γ-cyclodextrin polymer with one or more functionalizing agents (e.g., 2, 3 or 4), preferably with only one functionalizing agent; mixing α/β-cyclodextrin polymer with one or more functionalizing agents (e.g., 2, 3 or 4), preferably with only one functionalizing agent; mixing α/γ-cyclodextrin polymer with one or more functionalizing agents (e.g., 2, 3 or 4), preferably with only one functionalizing agent; mixing β/γ-cyclodextrin polymer with one or more functionalizing agents (e.g., 2, 3 or 4), preferably with only one functionalizing agent; or mixing α/β/γ-cyclodextrin polymer with one or more functionalizing agents (e.g., 2, 3 or 4), preferably with only one functionalizing agent; each optionally in the presence of at least one solvent.

The inventive copolymers can preferably also be obtained by a process comprising the process step of mixing (combining) any mixtures of the aforementioned cyclodextrin polymers with at least one functionalizing agent. It is advantageous that the inventive copolymers can be obtained by mixing (combining) mixtures containing α- and β-cyclodextrin polymer, α- and γ-cyclodextrin polymer, β- and γ-cyclodextrin polymer or α-, β- and γ-cyclodextrin polymer with one or more functionalizing agents, preferably with only one functionalizing agent, optionally in the presence of at least one solvent.

In a preferred embodiment, at least one, preferably only one cyclodextrin polymer is mixed with at least one, preferably only one functionalizing agent, optionally in the presence of at least one solvent; where the functionalizing agent is selected from the group consisting of C₁₋₆-alkyl-Y, C₁₋₆-alkyl-C(═O)—Y, C₃₋₇-cycloalkyl-C(═O)—Y, Y—C₁₋₆-alkyl-C(═O)—OH, Y—C₃₋₇-cycloalkyl-C(═O)—OH, Y—C₁₋₆-alkyl-C(═O)—O—C₁₋₆-alkyl, Y—C₃₋₇-cycloalkyl-C(═O)—O—C₃₋₇-cycloalkyl, [C₁₋₆-alkyl-C(═O)]₂O, [C₃₋₇-cycloalkyl-C(═O)]₂O, dihydrofuran-2,5-dione, furan-2,5-dione, dihydro-2H-pyran-2,6(3H)dione, oxirane, C₁₋₆-alkyloxirane, C₃₋₇-cycloalkyloxirane, di-C₁₋₆-alkyloxirane, di-C₃₋₇-cycloalkyloxirane, 2-chloroethanol, di-C₁₋₆-alkyl sulfate, di-C₃₋₇-cycloalkyl sulfate, di-C₁₋₆-alkyl phosphorochloridate, di-C₃₋₇-cycloalkyl phosphorochloridate, NH₃, (C₁₋₆-alkyl)NH₂, (C₃₋₇-cycloalkyl)NH₂, (C₁₋₆-alkyl)₂NH, (C₃₋₇-cycloalkyl)₂NH, (C₁₋₆-alkyl)₃N, (C₃₋₇-cycloalkyl)₃N, [(Y—C₁₋₆-alkyl)N(C₁₋₆-alkyl)₃]⁺ halide, 1,3-propane sultone and 1,4-butane sultone; where C₁₋₆-alkyl and C₃₋₇-cycloalkyl, independently of one another, may be unsubstituted or mono- or polysubstituted with the same or different Z radicals; where the additional substituents Z result in little or no significant crosslinking of the cyclodextrin monomers; halide preferably stands for chloride, bromide or iodide, especially preferably chloride or bromide and in particular chloride; and the Y radicals stand for leaving groups; where the leaving groups Y, independently of one another, are preferably selected from the group consisting of —Cl, —Br, —I, -mesyl, -tosyl and epoxy; especially preferably selected from the group consisting of —Cl, —Br, —I, -mesyl and -tosyl, and in particular for —Cl; and the substituents Z, independently of one another, are preferably selected from the group consisting of —OH, ═O, —C(═O)OH, —C(═O)OCH₃, —C(═O)OCH₂CH₃, —NH₂, —NH(CH₃), —N(CH₃)₂, —N(CH₃)₃⁺A⁻, —NH (CH₂CH₃), —N(CH₂CH₃)₂ and —N(CH₂CH₃)₃⁺A⁻; especially preferably from the group consisting of —OH, ═O, —C(═O)OH, —C(═O)OCH₃ and —C(═O)OCH₂CH₃; and in particular are selected from the group consisting of —OH, ═O and —C(═O)OH; where A⁻ stands for an organic or inorganic anion, which is preferably selected from the group consisting of Cl⁻, Br⁻, I⁻, dihydrogen phosphate, hydrogen phosphate, phosphate, bicarbonate, carbonate, acetate, lactate and citrate or for other physiologically tolerable anions.

In another preferred embodiment, at least one, preferably only one cyclodextrin polymer is mixed with at least one, preferably only one functionalizing agent, optionally in the presence of at least one solvent; where the functionalizing agent is selected from the group consisting of CH₃—Y¹, CH₃—CH₂—Y¹, CH₃—CH₂—CH₂—Y¹, (CH₃)₂CH—Y¹, CH₃—CH₂—CH₂—CH₂—Y¹, (CH₃)₂CH—CH₂—Y¹, CH₃—CH₂—CH(CH₃)—Y¹, (CH₃)₃C—Y¹, CH₂═CH—Y¹, CH₂═CH—CH₂—Y¹, cyclopropyl-Y¹, Y¹—CH₂—C(═O)—OH, Y¹—CH₂—CH₂—C(═O)—OH, Y¹—CH(CH₃)—C(═O)—OH, Y¹—CH₂—C(═O)—O—CH₃, Y¹—CH₂—CH₂—C(═O)—O—CH₃, Y¹—CH(CH₃)—C(═O)—O—CH₃, Y¹—CH₂—C(═O)—O—CH₂CH₃, Y¹—CH₂—CH₂—C(═O)—O—CH₂—CH₃, Y¹—CH(CH₃)—C(═O)—O—CH₂—CH₃, CH₃—C(═O)Y², CH₃—CH₂—C(═O)Y², (CH₃)₂CH—C(═O)Y², CH₃—CH₂—CH₂—C(═O)Y², (CH₃)₃C—C(═O)Y², CH₃—CH₂—CH(CH₃)—C(═O)Y², (CH₃)₂CH—CH₂—C(═O)Y², CH₂═CH—C(═O)Y², CH₂═CH—CH₂—C(═O)Y², cyclopropyl-C(═O)Y², dihydrofuran-2,5-dione, furan-2,5-dione, dihydro-2H-pyran-2,6(3H)dione, [CH₃—C(═O)]₂O, [CH₃—CH₂—C(═O)]₂O, [CH₃—CH₂—CH₂—C(═O)]₂O, [(CH₃)₂CH—C(═O)]₂O, [CH₃—CH₂—CH₂—CH₂—C(═O)]₂O, [CH₃—CH₂—CH(CH₃)—C(═O)]₂O, [(CH₃)₂CH—CH₂—C(═O)]₂O, [(CH₃)₃C—C(═O)]₂O, [CH₂═CH—C(═O)]₂O, [CH₂═CH—CH₂—C(═O)]₂O, [cyclopropyl-C(═O)]₂O, CH₂═CH—C(═O)—OH, CH₃—CH═CH—C(═O)—OH, (CH₃)₂C═CH—C(═O)—OH, CH₃—CH₂—CH═CH—C(═O)—OH, CH₃—CH═C(CH₃)—C(═O)—OH, CH₂═CH—C(═O)—O—CH₃, CH₃—CH═CH—C(═O)—O—CH₃, (CH₃)₂C═CH—C(═O)—O—CH₃, CH₃—CH₂—CH═CH—C(═O)—O—CH₃, CH₃—CH═C(CH₃)—C(═O)—O—CH₃, CH₂═CH—C(═O)—O—CH₂—CH₃, CH₃—CH═CH—C(═O)—O—CH₂—CH₃, (CH₃)₂C═CH—C(═O)—O—CH₂—CH₃, CH₃—CH₂—CH═CH—C(═O)—O—CH₂—CH₃, CH₃—CH═C(CH₃)—C(═O)O—CH₂—CH₃, oxirane, 2-methyloxirane, 2-ethyloxirane, oxiran-2-yl-methanol, oxirane-2-carboxylic acid, oxirane-2-carboxylic acid methyl ester, oxirane-2-carboxylic acid ethyl ester, 2-(oxiran-2-yl)acetic acid, 2-(oxirane-2-yl)acetic acid methyl ester, 2-(oxiran-2-yl)acetic acid ethyl ester, Cl—CH₂—CH₂—OH, (CH₃—O)₂S(═O)₂, (CH₃—CH₂—O)₂S(═O)₂, (CH₃—O)₂P(═O)Cl, (CH₃—CH₂-0)₂P(═O)Cl, [(CH₃)₂CH—O]₂P(═O)Cl, NH₃, CH₃—NH₂, (CH₃)₂NH, (CH₃)₃N, CH₃—CH₂—NH₂, (CH₃—CH₂)₂NH, (CH₃—CH₂)₃N, [Cl—CH₂—CH₂—N(CH₃)₃]⁺Cl⁻, [Cl—CH₂—CH₂—N(CH₂—CH₃)₃]⁺Cl⁻, 1,3-propane sultone and 1,4-butane sultone; where the Y¹ radicals, independently of one another, preferably stand for —Cl, —Br, —I, -tosyl, -mesyl or epoxy, especially preferably stand for —Cl, —Br or epoxy and in particular for —Cl; and the Y² radicals, independently of one another, preferably stand for —Cl, —Br or —I, especially preferably —Cl or —Br and in particular —Cl.

In another preferred embodiment, only one cyclodextrin polymer is mixed with only one functionalizing agent, optionally in the presence of at least one solvent; where the functionalizing agent is selected from the group consisting of CH₃—Y¹, CH₃—CH₂—Y¹, cyclopropyl-Y¹, Y¹—CH₂—C(═O)—OH, Y¹—CH₂—C(═O)—O—CH₃, Y¹—CH₂—C(═O)—O—CH₂—CH₃, CH₃—C(═O)Y², dihydrofuran-2,5-dione, furan-2,5-dione, dihydro-2H-pyran-2,6(3H)-dione, [CH₃—C(═O)]₂O, [cyclopropyl-C(═O)]₂O, oxirane, 2-methyloxirane, 2-ethyloxirane, oxiran-2-yl-methanol, oxirane-2-carboxylic acid, oxirane-2-carboxylic acid methyl ester, oxirane-2-carboxylic acid ethyl ester, 2-(oxiran-2-yl)acetic acid, 2-(oxiran-2-yl)acetic acid methyl ester, 2-(oxiran-2-yl)acetic acid ethyl ester, Cl—CH₂—CH₂—OH, (CH₃—O)₂S(═O)₂, (CH₃—CH₂—O)₂S(═O)₂, CH₃—NH₂, (CH₃)₂NH, (CH₃)₃N, CH₃—CH₂—NH₂, (CH₃—CH₂)₂NH, (CH₃—CH₂)₃N, [Cl—CH₂—CH₂—N(CH₃)₃]⁺Cl⁻, [Cl—CH₂—CH₂—N(CH₂—CH₃)₃]⁺Cl⁻, 1,3-propane sultone and 1,4-butane sultone; where the Y¹ radicals, independently of one another, preferably stand for —Cl, —Br, —I, -tosyl, -mesyl or epoxy, especially preferably stand for —Cl, —Br or epoxy and in particular for —Cl; and the Y² radicals, independently of one another, preferably stand for —Cl, —Br or —I, especially preferably stand for —Cl or —Br and in particular for —Cl.

In another preferred embodiment, the inventive copolymers can be obtained by a process comprising the process step of:

mixing at least one, preferably only one cyclodextrin polymer selected from the group consisting of (glucosyl)-cyclodextrin copolymer; (galactosyl)-cyclodextrin copolymer; (maltosyl)-cyclodextrin copolymer; (maltriosyl)-cyclodextrin copolymer; (acetyl)cyclodextrin copolymer; (propionyl)-cyclodextrin copolymer; (Isobutyryl)-cyclodextrin copolymer; (butyryl)-cyclodextrin copolymer; (pivaloyl)-cyclodextrin copolymer; (pentanoyl)-cyclodextrin copolymer; (hexanoyl)-cyclodextrin copolymer; (cyclopropanecarbonyl)-cyclodextrin copolymer; (benzoyl)-cyclodextrin copolymer; (2-phenylacetyl)cyclodextrin copolymer; (2-carboxyacetyl)-cyclodextrin copolymer; (3-carboxypropanoyl)-cyclodextrin copolymer; (4-carboxybutanoyl)-cyclodextrin copolymer; (2-methoxy-2-oxoacetyl)-cyclodextrin copolymer; (3-methoxy-3-oxopropanoyl)-cyclodextrin copolymer; (4-methoxy-4-oxobutanoyl)-cyclodextrin copolymer; (2-hydroxyacetyl)cyclodextrin copolymer; (2-hydroxypropanoyl)-cyclodextrin copolymer; (3-hydroxypropanoyl)-cyclodextrin copolymer; (2-hydroxybutanoyl)-cyclodextrin copolymer; (3-hydroxybutanoyl)-cyclodextrin copolymer; (4-hydroxybutanoyl)-cyclodextrin copolymer; (2-hydroxypentanoyl)-cyclodextrin copolymer; (3-hydroxypentanoyl)-cyclodextrin copolymer; (4-hydroxypentanoyl)-cyclodextrin copolymer; (5-hydroxypentanoyl)cyclodextrin copolymer; (2,3-dihydroxypropanoyl)-cyclodextrin copolymer; (2,3-dihydroxybutanoyl)-cyclodextrin copolymer; (2,4-dihydroxybutanoyl)-cyclodextrin copolymer; (3,4-dihydroxybutanoyl)-cyclodextrin copolymer; (2,3-dihydroxypentanoyl)-cyclodextrin copolymer; (2,4-dihydroxypentanoyl)-cyclodextrin copolymer; (2.5-dihydroxypentanoyl)-cyclodextrin copolymer; (3,4-dihydroxypentanoyl)-cyclodextrin copolymer; (3,5-dihydroxypentanoyl)-cyclodextrin copolymer; (4,5-dihydroxypentanoyl)-cyclodextrin copolymer; (2,3,4-trihydroxybutanoyl)-cyclodextrin copolymer; (2,3,4-trihydroxypentanoyl)-cyclodextrin copolymer; (2,3,5-trihydroxypentanoyl)cyclodextrin copolymer; (2,4,5-trihydroxypentanoyl)-cyclodextrin copolymer; (3,4,5-trihydroxypentanoyl)-cyclodextrin copolymer; (2,3,4,5-tetrahydroxypentanoyl)-cyclodextrin copolymer; (2-aminoacetyl)-cyclodextrin copolymer; (2-aminopropanoyl)cyclodextrin copolymer; (2-aminobutanoyl)-cyclodextrin copolymer; [2-(1H-imidazol-1-yl)acetyl]-cyclodextrin copolymer; [3-(1H-imidazol-1-yl)propanoyl]-cyclodextrin copolymer; [4-(1H-imidazol-1-yl)butanoyl]-cyclodextrin copolymer; [2-(1H-imidazol-1-yl)acetyl]-cyclodextrin copolymer; [3-(1H-imidazol-1-yl)propanoyl]-cyclodextrin copolymer; [4-(1H-imidazol-1-yl) butanoyl]-cyclodextrin copolymer; [2-(pyridin-3-yl)acetyl]-cyclodextrin copolymer; [3-(pyridin-3-yl)propanoyl]-cyclodextrin copolymer; [4-(pyridin-3-yl)butanoyl]-cyclodextrin copolymer; (methyl)-cyclodextrin copolymer; (ethyl)-cyclodextrin copolymer; (isopropyl)-cyclodextrin copolymer; (N-propyl)-cyclodextrin copolymer; (isobutyl)-cyclodextrin copolymer; (N-butyl)-cyclodextrin copolymer; (sec-butyl)-cyclodextrin copolymer; (tert-butyl)-cyclodextrin copolymer; (cyclopropyl)-cyclodextrin copolymer; (phenyl)cyclodextrin copolymer; (benzyl)-cyclodextrin copolymer; (phenethyl)-cyclodextrin copolymer; (carboxymethyl)-cyclodextrin copolymer; (1-carboxyethyl)-cyclodextrin copolymer; (2-carboxyethyl)-cyclodextrin copolymer; (1-carboxypropyl)-cyclodextrin copolymer; (2-carboxypropyl)-cyclodextrin copolymer; (3-carboxypropyl)-cyclodextrin copolymer; (1-carboxypropan-2-yl)-cyclodextrin copolymer; (4-carboxybutyl)-cyclodextrin copolymer; (5-carboxypentyl)-cyclodextrin copolymer; (5-carboxypentyl)-cyclodextrin copolymer; (2-methoxy-2-oxoethyl)-cyclodextrin copolymer; (3-methoxy-2-oxopropyl)-cyclodextrin copolymer; (4-methoxy-4-oxobutyl)-cyclodextrin copolymer; (5-methoxy-5-oxopentyl)-cyclodextrin copolymer; (6-methoxy-6-oxohexyl)-cyclodextrin copolymer; (2-ethoxy-2-oxoethyl)cyclodextrin copolymer; (3-ethoxy-2-oxopropyl)-cyclodextrin copolymer; (4-ethoxy-4-oxobutyl)-cyclodextrin copolymer; (5-ethoxy-5-oxopentyl)-cyclodextrin copolymer; (6-ethoxy-6-oxohexyl)-cyclodextrin copolymer; (3-carboxy-2-hydroxypropyl)-cyclodextrin copolymer; (2-hydroxy-4-methoxy-4-oxobutyl)-cyclodextrin copolymer; (4-ethoxy-2-hydroxy-4-oxobutyl)-cyclodextrin copolymer; (1-carboxy-2-hydroxyethyl)-cyclodextrin copolymer; (3-hydroxy-1-methoxy-1-oxopropan-2-yl)-cyclodextrin copolymer; (1-ethoxy-3-hydroxy-1-oxopropan-2-yl)-cyclodextrin copolymer; (1-carboxy-3-hydroxypropan-2-yl)-cyclodextrin copolymer; (1-hydroxy-4-methoxy-4-oxobutan-2-yl)-cyclodextrin copolymer; (4-ethoxy-1-hydroxy-4-oxobutan-2-yl)-cyclodextrin copolymer; (hydroxymethyl)-cyclodextrin copolymer; (1-hydroxyethyl)-cyclodextrin copolymer; (2-hydroxyethyl)-cyclodextrin copolymer; (1-hydroxypropyl)-cyclodextrin copolymer; (2-hydroxypropyl)-cyclodextrin copolymer; (1-hydroxypropan-2-yl)-cyclodextrin copolymer; (2-hydroxypropan-2-yl)-cyclodextrin copolymer; (1-hydroxybutyl)-cyclodextrin copolymer; (2-hydroxybutyl)-cyclodextrin copolymer; (3-hydroxybutyl)-cyclodextrin copolymer; (4-hydroxybutyl)-cyclodextrin copolymer; (1-hydroxypentyl)-cyclodextrin copolymer; (2-hydroxypentyl)-cyclodextrin copolymer; (3-hydroxypentyl)-cyclodextrin copolymer; (4-hydroxypentyl)-cyclodextrin copolymer; (5-hydroxypentyl)-cyclodextrin copolymer; (1,2-dihydroxyethyl)-cyclodextrin copolymer; (1,2-dihydroxypropyl)-cyclodextrin copolymer; (1,3-dihydroxypropyl)-cyclodextrin copolymer; (2,3-dihydroxypropyl)-cyclodextrin copolymer; (1,2-dihydroxybutyl)-cyclodextrin copolymer; (1,3-dihydroxybutyl)-cyclodextrin copolymer; (1,4-dihydroxybutyl)-cyclodextrin copolymer; (2,3-dihydroxybutyl)-cyclodextrin copolymer; (2,4-dihydroxybutyl)-cyclodextrin copolymer; (3,4-dihydroxybutyl)-cyclodextrin copolymer; (1,2-dihydroxypentyl)-cyclodextrin copolymer; (1,3-dihydroxypentyl)-cyclodextrin copolymer; (1,4-dihydroxypentyl)cyclodextrin copolymer; (1,5-dihydroxypentyl)-cyclodextrin copolymer; (2,3-dihydroxypentyl)-cyclodextrin copolymer; (2,4-dihydroxypentyl)-cyclodextrin copolymer; (2.5-dihydroxypentyl)-cyclodextrin copolymer; (3,4-dihydroxypentyl)-cyclodextrin copolymer; (3,5-dihydroxypentyl)-cyclodextrin copolymer; (4,5-dihydroxypentyl)-cyclodextrin copolymer; (1,2,3-trihydroxypropyl)-cyclodextrin copolymer; (1,2,3-trihydroxybutyl)cyclodextrin copolymer; (1,2,4-trihydroxybutyl)-cyclodextrin copolymer; (1,3,4-trihydroxybutyl)-cyclodextrin copolymer; (2,3,4-trihydroxybutyl)-cyclodextrin copolymer; (1,2,3-trihydroxypentyl)-cyclodextrin copolymer; (1,2,4-trihydroxypentyl)-cyclodextrin copolymer; (1,2,5-trihydroxypentyl)-cyclodextrin copolymer; (1,3,4-trihydroxypentyl)-cyclodextrin copolymer; (1,3,5-trihydroxypentyl)-cyclodextrin copolymer; (1,4,5-trihydroxypentyl)-cyclodextrin copolymer; (2,3,4-trihydroxypentyl)-cyclodextrin copolymer; (2,3,5-trihydroxypentyl)-cyclodextrin copolymer; (2,4,5-trihydroxypentyl)cyclodextrin copolymer; (3,4,5-trihydroxypentyl)-cyclodextrin copolymer; (1,2,3,4-tetrahydroxybutyl)-cyclodextrin copolymer; (1,2,3,4-tetrahydroxypentyl)-cyclodextrin copolymer; (1,2,4,5-tetrahydroxypentyl)-cyclodextrin copolymer; (2,3,4,5-tetrahydroxypentyl)-cyclodextrin copolymer; (1,2,3,4,5-pentahydroxypentyl)-cyclodextrin copolymer; [(sulfoxy)methyl]-cyclodextrin copolymer; [2-(sulfoxy)ethyl]-cyclodextrin copolymer; [3-(sulfoxy)propyl]-cyclodextrin copolymer; [4-(sulfoxy)butyl]-cyclodextrin copolymer; [(phosphonooxy)methyl]-cyclodextrin copolymer; [2-(phosphonooxy)ethyl]-cyclodextrin copolymer; [3-(phosphonooxy)propyl]-cyclodextrin copolymer; [4-(phosphonooxy)butyl]-cyclodextrin copolymer; [4-(phosphonooxy)butyl]-cyclodextrin copolymer; (sulfomethyl)-cyclodextrin copolymer; (2-sulfoethyl)-cyclodextrin copolymer; (3-sulfopropyl)-cyclodextrin copolymer; (4-sulfobutyl)-cyclodextrin copolymer; (5-sulfopentyl)-cyclodextrin copolymer; (6-sulfohexyl)-cyclodextrin copolymer; (phosphonomethyl)-cyclodextrin copolymer; (2-phosphonoethyl)-cyclodextrin copolymer; (3-phosphonopropyl)-cyclodextrin copolymer; (4-phosphonobutyl)-cyclodextrin copolymer; (5-phosphonopentyl)-cyclodextrin copolymer; (6-phosphonohexyl)-cyclodextrin copolymer; (2-phosphonovinyl)-cyclodextrin copolymer; (3-phosphonoallyl)cyclodextrin copolymer; (4-phosphonobut-3-enyl)-cyclodextrin copolymer; (5-phosphonopent-4-enyl)-cyclodextrin copolymer; (6-phosphonohex-5-enyl)-cyclodextrin copolymer; (aminomethyl)-cyclodextrin copolymer; (2-aminoethyl)-cyclodextrin copolymer; (3-aminopropyl)-cyclodextrin copolymer; (4-aminobutyl)-cyclodextrin copolymer; (5-aminopentyl)-cyclodextrin copolymer; (6-aminohexyl)-cyclodextrin copolymer; [(N,N-dimethylamino)methyl]-cyclodextrin copolymer; [2-(N,N-dimethylamino)ethyl]-cyclodextrin copolymer; [3-(N,N-dimethylamino)propyl]-cyclodextrin copolymer; [4-(N,N-dimethylamino)butyl]-cyclodextrin copolymer; [5-(N,N-dimethylamino)pentyl]-cyclodextrin copolymer; [6-(N,N-dimethylamino) hexyl]-cyclodextrin copolymer; [(N,N-diethylamino)methyl]-cyclodextrin copolymer; [2-(N,N-diethylamino)ethyl]-cyclodextrin copolymer; [3-(N,N-diethylamino)propyl]-cyclodextrin copolymer; [4-(N,N-diethylamino)butyl]-cyclodextrin copolymer; [5-(N,N-diethylamino)pentyl]-cyclodextrin copolymer; [6-(N,N-diethylamino)hexyl]-cyclodextrin copolymer; [(trimethylammonio)methyl]-cyclodextrin copolymer chloride; [2-(trimethylammonio)ethyl]-cyclodextrin copolymer chloride; [3-(trimethylammonio)propyl]-cyclodextrin copolymer chloride; [4-(trimethylammonio)butyl]-cyclodextrin copolymer chloride; [5-(trimethylammonio)pentyl]-cyclodextrin copolymer chloride; [6-(trimethylammonio)hexyl]-cyclodextrin copolymer chloride; [(triethylammonio)methyl]-cyclodextrin copolymer chloride; [2-(triethylammonio)ethyl]-cyclodextrin copolymer chloride; [3-(triethylammonio)propyl]-cyclodextrin copolymer chloride; [4-(triethylammonio)butyl]-cyclodextrin copolymer chloride; [5-(triethylammonio)pentyl]-cyclodextrin copolymer chloride; [6-(triethylammonio)hexyl]-cyclodextrin copolymer chloride; [(1H-imidazol-1-yl)methyl]-cyclodextrin copolymer; [2-(1H-imidazol-1-yl)ethyl]-cyclodextrin copolymer; [3-(1H-imidazol-1-yl)propyl]-cyclodextrin copolymer; [4-(1H-imidazol-1-yl)butyl]-cyclodextrin copolymer; (pyridin-3-ylmethyl)-cyclodextrin copolymer; [2-(pyridin-3-yl)ethyl]-cyclodextrin copolymer; [3-(pyridin-3-yl)propyl]-cyclodextrin copolymer; [4-(pyridin-3-yl)butyl]-cyclodextrin copolymer; [β-D-glucopyranosyloxyuronic acid) methyl]-cyclodextrin copolymer; [2-(β-D-glucopyranosyloxyuronic acid) ethyl]-cyclodextrin copolymer; [3-(β-D-glucopyranosyloxyuronic acid) propyl]-cyclodextrin copolymer; [4-(β-D-glucopyranosyloxyuronic acid)butyl]-cyclodextrin copolymer; [5-(β-D-glucopyranosyloxyuronic acid) pentyl]-cyclodextrin copolymer; and [6-(β-D-glucopyranosyloxyuronic acid) hexyl]-cyclodextrin copolymer; where the term “cyclodextrin copolymer” stands for α-, β-, γ, α/β-, α/γ-, β/γ or α/β/γ-cyclodextrin copolymer, preferably for α-, β- or γ-cyclodextrin copolymer, more preferably for β-cyclodextrin copolymer; and in particular for α-cyclodextrin-epichlorohydrin copolymer, β-cyclodextrin-epichlorohydrin copolymer, γ-cyclodextrin-epichlorohydrin copolymer, α-cyclodextrin-4-chloro-1,2-epoxybutane copolymer, β-cyclodextrin-4-chloro-1,2-epoxybutane copolymer, γ-cyclodextrin-4-chloro-1,2-epoxybutane copolymer, α-cyclodextrin-1,2,3,4-diepoxybutane copolymer, β-cyclodextrin-1,2,3,4-diepoxybutane copolymer, γ-cyclodextrin-1,2,3,4-diepoxybutane copolymer, α-cyclodextrin-tetramethylene diisocyanate copolymer, β-cyclodextrin-tetramethylene diisocyanate copolymer, γ-cyclodextrin-tetramethylene diisocyanate copolymer, α-cyclodextrin-hexamethylene diisocyanate copolymer, β-cyclodextrin-hexamethylene diisocyanate copolymer or γ-cyclodextrin-hexamethylene diisocyanate copolymer;
with at least one, preferably only one functionalizing agent selected from the group consisting of CH₃—Y¹, CH₃—CH₂—Y¹, cyclopropyl-Y¹, Y¹—CH₂—C(═O)—OH, Y¹—CH₂—C(═O)—O—CH₃, Y¹—CH₂—C(═O)—O—CH₂—CH₃, CH₃—C(═O)Y², dihydrofuran-2,5-dione, furan-2,5-dione, dihydro-2H-pyran-2,6(3H)-dione, [CH₃—C(═O)]₂O, [cyclopropyl-C(═O)]₂O, oxirane, 2-methyloxirane, 2-ethyloxirane, oxiran-2-yl-methanol, oxirane-2-carboxylic acid, oxirane-2-carboxylic acid methyl ester, oxirane-2-carboxylic acid ethyl ester, 2-(oxiran-2-yl) acetic acid, 2-(oxiran-2-yl)acetic acid methyl ester, 2-(oxiran-2-yl)acetic acid ethyl ester, Cl—CH₂—CH₂—OH, (CH₃—O)₂S(═O)₂, (CH₃—CH₂—O)₂S(═O)₂, CH₃—NH₂, (CH₃)₂NH, (CH₃)₃N, CH₃—CH₂—NH₂, (CH₃—CH₂)₂NH, (CH₃—CH₂)₃N, [Cl—CH₂—CH₂—N(CH₃)₃]⁺Cl⁻, [Cl—CH₂—CH₂—N(CH₂—CH₃)₃]⁺Cl⁻, 1,3-propane sultone and 1,4-butane sultone; where the Y¹ radicals, independently of one another, preferably stand for —Cl, —Br, —I, -tosyl, -mesyl or epoxy, especially preferably stand for —Cl, —Br or epoxy and in particular —Cl; and Y² radicals preferably stand for —Cl, —Br or —I, especially preferably —Cl or —Br and in particular —Cl; optionally in the presence of at least one solvent.

The inventive copolymers can preferably be obtained by a process comprising the process step of mixing
unsubstituted α-cyclodextrin-epichlorohydrin copolymer;
unsubstituted α-cyclodextrin-4-chloro-1,2-epoxybutane copolymer;
unsubstituted α-cyclodextrin-1,2,3,4-diepoxybutane copolymer;
unsubstituted β-cyclodextrin-epichlorohydrin copolymer;
unsubstituted β-cyclodextrin-1,2,3,4-diepoxybutane copolymer;
unsubstituted γ-cyclodextrin-epichlorohydrin copolymer;
unsubstituted γ-cyclodextrin-4-chloro-1,2-epoxybutane copolymer;
unsubstituted γ-cyclodextrin-1,2,3,4-diepoxybutane copolymer; or
a mixture containing at least two different cyclodextrin polymers of those listed above;
   with a functionalizing agent, where the functionalizing agent is preferably selected from the group consisting of CH₃—Y¹, CH₃—CH₂—Y¹, cyclopropyl-Y¹, Y¹—CH₂—C(═O)—OH, Y¹—CH₂—C(═O)—O—CH₃, Y¹—CH₂—C(═O)—O—CH₂—CH₃, CH₃—C(═O)Y², dihydrofuran-2,5-dione, furan-2,5-dione, dihydro-2H-pyran-2,6(3H)-dione, [CH₃—C(═O)]₂O, [cyclopropyl-C(═O)]₂O, oxirane, 2-methyloxirane, 2-ethyloxirane, oxiran-2-yl-methanol, oxirane-2-carboxylic acid, oxirane-2-carboxylic acid methyl ester, oxirane-2-carboxylic acid ethyl ester, 2-(oxiran-2-yl)acetic acid, 2-(oxiran-2-yl)acetic acid methyl ester, 2-(oxiran-2-yl) acetic acid ethyl ester, Cl—CH₂—CH₂—OH, (CH₃—O)₂S(═O)₂, (CH₃—CH₂—O)₂S(═O)₂, CH₃—NH₂, (CH₃)₂NH, (CH₃)₃N, CH₃—CH₂—NH₂, (CH₃—CH₂)₂NH, (CH₃—CH₂)₃N, [Cl—CH₂—CH₂—N(CH₃)₃]⁺Cl⁻, [Cl—CH₂—CH₂—N(CH₂—CH₃)₃]⁺Cl⁻, 1,3-propane sultone and 1,4-butane sultone; where the Y¹ radicals, independently of one another, preferably stand for —Cl, —Br, —I, -tosyl, -mesyl or epoxy, especially preferably stand for —Cl, —Br or epoxy and in particular for —Cl; and Y² radicals preferably stand for —Cl, —Br or —I, especially preferably stand for —Cl or —Br and in particular for —Cl; optionally in the presence of at least one solvent.

In another preferred embodiment, the inventive copolymers can be obtained by a process comprising the process step of mixing:
unsubstituted α-cyclodextrin-epichlorohydrin copolymer with 1,3-propane sultone, 1,4-butane sultone, chloroacetic acid, chloroacetic acid methyl ester or chloroacetic acid ethyl ester;
unsubstituted α-cyclodextrin-4-chloro-1,2-epoxybutane copolymer with 1,3-propane sultone, 1,4-butane sultone, chloroacetic acid, chloroacetic acid methyl ester or chloroacetic acid ethyl ester;
unsubstituted α-cyclodextrin-1,2,3,4-diepoxybutane copolymer with 1,3-propane sultone, 1,4-butane sultone, chloroacetic acid, chloroacetic acid methyl ester or chloroacetic acid ethyl ester;
unsubstituted β-cyclodextrin-epichlorohydrin copolymer with 1,3-propane sultone, 1,4-butane sultone, chloroacetic acid, chloroacetic acid methyl ester or chloroacetic acid ethyl ester;
unsubstituted β-cyclodextrin-4-chloro-1,2-epoxybutane copolymer with 1,3-propane sultone, 1,4-butane sultone, chloroacetic acid, chloroacetic acid methyl ester or chloroacetic acid ethyl ester;
unsubstituted β-cyclodextrin-1,2,3,4-diepoxybutane copolymer with 1,3-propane sultone, 1,4-butane sultone, chloroacetic acid, chloroacetic acid methyl ester or chloroacetic acid ethyl ester;
unsubstituted γ-cyclodextrin-epichlorohydrin copolymer with 1,3-propane sultone, 1,4-butane sultone, chloroacetic acid, chloroacetic acid methyl ester or chloroacetic acid ethyl ester;
unsubstituted γ-cyclodextrin-4-chloro-1,2-epoxybutane copolymer with 1,3-propane sultone, 1,4-butane sultone, chloroacetic acid, chloroacetic acid methyl ester or chloroacetic acid ethyl ester; or
unsubstituted γ-cyclodextrin-1,2,3,4-diepoxybutane copolymer with 1,3-propane sultone, 1,4-butane sultone, chloroacetic acid, chloroacetic acid methyl ester or chloroacetic acid ethyl ester;
   optionally in the presence of at least one solvent.

In another preferred embodiment, an unsubstituted α-. 11- or γ-cyclodextrin-epichlorohydrin copolymer is mixed with only one functionalizing agent from the group of 1,3-butane sultone, 1,4-butane sultone, 2-chloroacetic acid, 2-chloroacetic acid methyl ester and 2-chloroacetic acid ethyl ester; optionally in the presence of a solvent.

The mixing of the cyclodextrin polymers with functionalizing agents may be performed in aqueous or organic solvents. The mixing is preferably performed in water, dimethyl sulfoxide (DMSO), dimethylformamide (DMF), N-methyl-2-pyrrolidone (NMP) or mixtures thereof. The reaction may also be performed in anhydrous DMSO, anhydrous DMF, anhydrous NMP or mixtures thereof, preferably under anhydrous conditions. The solvents are advantageously water, DMSO and DMF as well as mixtures thereof, in particular water or DMF as well as mixtures thereof.

The mixing is preferably performed in an alkaline medium, usually at a pH of >7.

The mixing is preferably performed at 7.5≦pH≦14, more preferably at 7.5≦pH≦13, even more preferably at 8≦pH≦13, most preferably at 8≦pH≦12.5 and in particular at 9≦pH≦12.

The pH of the reaction solution or dispersion is preferably adjusted using bases for example, basic alkali metal salts, basic alkaline earth metal salts, hydrides, alkoxides or organic bases, for example, pyridine or diisopropylamine. Sodium hydroxide, sodium bicarbonate, sodium carbonate, potassium hydroxide, potassium bicarbonate, potassium carbonate, sodium hydride, potassium hydride, sodium methanolate, potassium-tert-butoxide, pyridine and diisopropylamine.

Since the cyclodextrin polymers are functionalized under basic reaction conditions, substituents which are not stable in an alkaline medium may be structurally modified. This modification may involve base-labile groups of the cyclodextrin polymers, cross-linking agents and functionalizing agents. For example, the ester group of the cyclodextrin polymer (2-methoxy-2-oxoethyl)-cyclodextrin-epichlorohydrin copolymer may be cleaved to form a carboxyl or carboxylate group, where the (2-methoxy-2-oxoethyl)-cyclodextrin-epichlorohydrin copolymer is converted to (carboxymethyl)-cyclodextrin-epichlorohydrin copolymer. This conversion may be complete or incomplete, based on the total number of ester groups. Consequently, all or only some of the ester groups may be converted to carboxyl groups in the corresponding copolymer according to the invention.

In a preferred embodiment, the cyclodextrin polymers are dissolved or dispersed in a solvent or solvent mixture and then the functionalizing agents are dissolved or dispersed therein, preferably while stirring the dispersion or solution. The functionalizing agent may be added all at once or successively in portions. The functionalizing agents may be dissolved or dispersed in a solvent (e.g., water, DMF, DMSO, NMP) and added as a solution or dispersion to the reaction mixture all once or successively in portions or added by drops by means of a dropping funnel, a pipette or a syringe. The addition or dropwise addition may take place with the help of a syringe pump.

In another preferred embodiment, the functionalizing agents are dissolved or dispersed in a solvent or solvent mixture and then, preferably while stirring the solution or dispersion, the cyclodextrin polymers are dissolved or dispersed in this solution. The cyclodextrin polymers may be added all at once or successively in portions. The cyclodextrin polymers are dissolved or dispersed in a solvent before being added and are added as a solution or dispersion to the reaction mixture all at once or successively in portions or are added by drops by means of a dropping funnel, a pipette or a syringe. The addition or dropwise addition may take place with the help of a syringe pump.

In another preferred embodiment, the functionalizing agents and cyclodextrin polymers are dissolved or dispersed simultaneously in a solvent or solvent mixture.

In mixing there may be a sudden and unwanted increase in the reaction temperature. It is therefore advantageous to cool the reaction mixture optionally to ensure an essentially constant reaction temperature. The reaction mixture may be cooled in an ice bath or a water bath.

The reaction mixture can be alkalized before, during or after addition of the cyclodextrin polymers, before, during or after addition of the functionalizing agents or before, during or after addition of a mixture thereof.

The alkaline pH is preferably measured with the help of pH paper or a pH meter. In a preferred embodiment, the pH is measured continuously or at certain intervals during the reaction period. If the pH drops below a certain level (e.g., pH<8 or <7.5), an alkaline pH can be restored in the reaction mixture by adding a base.

The reaction is preferably performed at a reaction temperature (temperature of the reaction mixture) of 5.0 to 90° C., more preferably 10 to 75° C., even more preferably 15 to 50° C., most preferably 15 to 30° C. and in particular 20 to 25° C. The temperature of the reaction mixing is preferably set at 5 to 90° C., more preferably at 10 to 75° C., even more preferably at 15 to 50° C., most preferably at 15 to 30° C. and in particular at 20 to 25° C., before, during or after mixing the cyclodextrin polymers with the functionalizing agents, optionally in the presence of a solvent or solvent mixture.

In a preferred embodiment, a total substance quantity of ≧5.0 mol, ≧10 mol, ≧20 mol or ≧30 mol, more preferably ≧40 mol, ≧50 mol or ≧60 mol, even more preferably ≧70 mol, ≧80 mol or ≧90 mol, most preferably ≧100 mol, ≧150 mol or ≧200 mol, and in particular ≧250 mol of functionalizing agent, based on a total substance quantity of 1.0 mol of cyclodextrin polymers, is used in the reaction mixture (equivalents).

In another preferred embodiment, the ratio of the total substance quantity of functionalizing agents to the total substance quantity of cyclodextrin polymers is ≧1:1, more preferably ≧10:1, even more preferably ≧25:1, most preferably ≧50:1 and in particular ≧100:1.

The reaction may be followed by suitable analytical processes. For example, random samples of the reaction mixture may be taken at certain points in time and analyzed by LC/MS or NMR. In this way, the reaction product present in the reaction mixture at a certain point in time can be determined. If the random sample analysis reveals that the desired reaction product is present, the reaction may be terminated and the desired reaction product may be isolated and/or purified.

To terminate the reaction, the reaction mixture is preferably neutralized, i.e., the pH of the reaction solution or dispersion is preferably set at 6.5≦pH≦7.5, especially preferably at 6.8≦pH≦7.2. The mixture is preferably neutralized with organic acids (e.g., trifluoroacetic acid, acetic acid) or inorganic acids (e.g., hydrochloric acid, sulfuric acid, nitric acid).

In another preferred embodiment, after the reaction and/or neutralization, the reaction mixture is preferably dialyzed in a salt-free process, in which the dialysis membrane used in this dialysis process has an MWCO (“molecular weight cut-off”) of preferably 0.50 kDa or 1.0 kDa, more preferably of 2.0 kDa or 3.0 kDa, even more preferably of 4.0 kDa or 5.0 kDa, most preferably of 10 kDa and in particular ≧15 kDa.

The salt-free dialyzed inventive copolymer preferably has a residual content of compounds having a molecular weight of <1.5 kDa, based on the total weight of inventive copolymer of ≦10 wt %, more preferably ≦5.0 wt %, even more preferably ≦2.5 wt % and in particular ≦1.0 wt %.

The reaction mixture and/or the essentially salt-free dialysis solution may be concentrated preferably with the help of a rotary evaporator, before further workup and/or isolation. The reaction mixture may also be concentrated already before dialysis, preferably salt-free dialysis.

The Isolation or purification of the inventive copolymers is preferably performed by means of ultrafiltration, column chromatography (e.g., on silica gel or activated carbon), preparative HPLC or recrystallization. The resulting inventive copolymer may be lyophilized.

In a preferred embodiment, the substance quantity proportion of the linkers contained in the inventive copolymer amounts to 20 to 95 mol %, more preferably 25 to 80 mol %, even more preferably 30 to 70 mol %, most preferably 30 to 60 mol % and in particular 30 to 50 mol %, based on the total substance quantity of the monomers contained in the inventive copolymer.

The substance quantity proportion of the cyclodextrin monomers contained in the inventive copolymer preferably amounts to 5.0 to 80 mol %, more preferably 10 to 75 mol %, even more preferably 20 to 70 mol %, most preferably 30 to 70 mol % and in particular 40 to 70 mol %, based on the total substance quantity of the monomers contained in the inventive copolymer.

In another preferred embodiment, the sum of the substance quantity proportion of the linkers and the substance quantity proportion of the cyclodextrin monomers is ≧25 mol %, ≧30 mol % or ≧40 mol %, more preferably ≧50 mol % or ≧60 mol %, even more preferably ≧70 mol % or ≧80 mol %, most preferably ≧90 mol % or ≧95 mol %, and in particular 100 mol %, based on the total substance quantity of the monomers contained in the inventive copolymer.

The water solubility of the inventive copolymers at 23° C. is preferably ≧5.0 g l⁻¹, ≧10 g l⁻¹, ≧25 g l⁻¹ or ≧50 g l⁻¹, more preferably ≧100 g l⁻¹, ≧125 g l⁻¹ or ≧150 g l⁻¹, even more preferably ≧175 g l⁻¹, ≧200 g l⁻¹, ≧225 g l⁻¹ or ≧250 g l⁻¹, most preferably ≧300 g l⁻¹ or ≧400 g l⁻¹ and in particular ≧500 g l⁻¹.

The osmolality of a 50 mM aqueous solution of inventive copolymer is preferably ≧50 mosm kg⁻¹, ≧75 mosm kg⁻¹ or ≧100 mosm kg⁻¹, more preferably ≧125 mosm kg⁻¹, ≧150 mosm kg⁻¹ or ≧175 mosm kg⁻¹, even more preferably ≧200 mosm kg⁻¹ or ≧225 mosm kg⁻¹, most preferably ≧250 mosm kg⁻¹ or ≧275 mosm kg⁻¹ and in particular ≧300 mosm kg⁻¹.

The degree of polymerization of the inventive copolymers is preferably ≧2.0 or ≧3.0, more preferably ≧4.0 or ≧5.0, even more preferably ≧6.0 or ≧7.0, most preferably ≧8.0 or ≧9.0 and in particular ≧10.

The average molecular weight of the inventive copolymers is preferably ≧2.0 kDa, more preferably ≧2.5 kDa, even more preferably ≧3.0 kDa, most preferably ≧4.0 kDa and in particular ≧5.0 kDa. The average molecular weight is preferably measured with the help of a membrane osmometer (distilled water, 23° C.).

In another preferred embodiment, the average molecular weight (M) of the inventive polymers is preferably 2.0≦M≦100 kDa, more preferably 2.0≦M≦50 kDa, even more preferably 2.0≦M≦25 kDa, most preferably 2.0≦M≦15 kDa and in particular 2.0≦M≦10 kDa.

In another especially preferred embodiment, the inventive copolymer preferably has a degree of polymerization of ≧2.0 or ≧3.0, more preferably ≧4.0 or ≧5.0, even more preferably ≧6.0 or ≧7.0, most preferably ≧8.0 or ≧9.0 and in particular ≧10.

In another preferred embodiment, the average particle diameter of the inventive copolymers is ≦1000 μm, ≦900 μm or ≦800 μm, more preferably ≦700 μm, ≦600 μm or ≦500 μm, even more preferably ≦400 μm, ≦300 μm or ≦200 μm, most preferably ≦100 μm, ≦75 μm or ≦50 μm and in particular ≦25 μm.

In a preferred embodiment, the inventive copolymers are suitable for use in the treatment of renal insufficiency.

In another preferred embodiment, the inventive copolymers are suitable for use in dialysis treatment.

In another preferred embodiment, the inventive copolymers are suitable for use in hemodialysis and/or peritoneal dialysis treatment because of their osmotic efficacy, among other things.

Another subject matter of this invention relates to dialysis solutions containing at least one inventive copolymer.

In a preferred embodiment, the inventive dialysis solution is a hemodialysis solution or a peritoneal dialysis solution.

Dosage forms used in dialysis treatment are preferably concentrates in multicomponent systems or ready-to-use dialysis solutions.

For the purposes of this invention, the term “dialysis solution” includes a ready-to-use dosage form for dialysis treatment, i.e., a liquid preparation, which is suitable for administration as such. In particular the dialysis solution need not be diluted and/or mixed with other preparations prior to administration.

In contrast with the dialysis solutions described above, concentrates which may be in liquid, semisolid or solid form, are diluted with water or aqueous solutions prior to administration or are dissolved in water or aqueous solutions. Similarly, the components of a multicomponent solution must be mixed with one another prior to administration in order to obtain a ready-to-use dialysis solution. Concentrates and multicomponent systems may thus be regarded as the precursor to the inventive dialysis solution.

The inventive dialysis solution is preferably a hemodialysis solution or a peritoneal dialysis solution. Hemodialysis and peritoneal dialysis solutions usually contain electrolytes in a concentration corresponding essentially to the plasma electrolyte concentration. The electrolytes usually include sodium, potassium, calcium, magnesium and chloride ions.

Dialysis solutions usually have a physiologically tolerable pH. This is preferably achieved by using buffers (buffer systems), which may themselves contribute to the total electrolyte content. The buffers are preferably bicarbonate, lactate or pyruvate.

Furthermore, dialysis solutions usually have a physiologically tolerable osmolarity. This is usually achieved by the electrolytes contained in the dialysis solution and inventive copolymers which are physiologically tolerable as osmotically active substances (osmotics) in the desired concentration.

The inventive dialysis solution has an osmolarity in the range of preferably 200 to 550 mosm l⁻¹.

In the case when the inventive dialysis solution is a hemodialysis solution, the osmolarity is preferably 200 to 350 mosm l⁻¹ or 210 to 340 mosm l⁻¹, more preferably 220 to 330 mosm l⁻¹, even more preferably 230 to 320 mosm l⁻¹, most preferably 240 to 310 mosm l⁻¹ and in particular 250 to 300 mosm l⁻¹. Processes for measuring the osmolarity and the osmotic pressure are familiar to the person skilled in the art. For example, they may be determined with the help of a membrane osmometer.

In the case when the inventive dialysis solution is a peritoneal dialysis solution, the osmolarity is preferably 200 to 570 mosm l⁻¹ or 210 to 560 mosm l⁻¹, more preferably 220 to 550 mosm l⁻¹, even more preferably 230 to 540 mosm l⁻¹, most preferably 240 to 530 mosm l⁻¹ and in particular 250 to 520 mosm l⁻¹. In a preferred embodiment, the osmolarity is 250±50 mosm l⁻¹ or 250±45 mosm l⁻¹, more preferably 250±35 mosm l⁻¹, even more preferably 250±25 mosm l⁻¹, most preferably 250±15 mosm l⁻¹ and in particular 250±10 mosm l⁻¹. In another preferred embodiment, the osmolarity is 300±50 mosm l⁻¹ or 300±45 mosm l⁻¹, more preferably 300±35 mosm l⁻¹, even more preferably 300±25 mosm l⁻¹, most preferably 300±15 mosm l⁻¹ and in particular 300±10 mosm l⁻¹. In another preferred embodiment, the osmolarity is 350±50 mosm l⁻¹ or 350±45 mosm l⁻¹, more preferably 350±35 mosm l⁻¹, even more preferably 350±25 mosm l⁻¹, most preferably 350±15 mosm l⁻¹ and in particular 300±10 mosm l⁻¹. In another preferred embodiment, the osmolarity is 400±50 mosm l⁻¹ or 400±45 mosm l⁻¹, more preferably 400±35 mosm l⁻¹, even more preferably 400±25 mosm l⁻¹, most preferably 400±15 mosm l⁻¹ and in particular 300±10 mosm l⁻¹. In another preferred embodiment, the osmolarity is 450±50 mosm l⁻¹ or 450±45 mosm l⁻¹, more preferably 450±35 mosm l⁻¹, even more preferably 450±25 mosm l⁻¹, most preferably 450±15 mosm l⁻¹ and in particular 450±10 mosm C. In another preferred embodiment, the osmolarity is 500±50 mosm l⁻¹ or 500±45 mosm l⁻¹, more preferably 500±35 mosm l⁻¹, even more preferably 500±25 mosm l⁻¹, most preferably 500±15 mosm l⁻¹ and in particular 500±10 mosm l⁻¹.

The inventive dialysis solution preferably has a pH of 4.9 to 8.0, more preferably 5.2 to 7.8, measured at room temperature (20 to 23° C.). In a preferred embodiment, the pH is 5.5±1.0 or 5.5±0.8, more preferably 5.5±0.7 or 5.5±0.6, even more preferably 5.5±0.5 or 5.5±0.4, most preferably 5.5±0.3 or 5.5±0.2 and in particular 5.5±0.1. In another preferred embodiment, the pH is 6.0±1.0 or 6.0±0.8, more preferably 6.0±0.7 or 6.0±0.6, even more preferably 6.0±0.5 or 6.0±0.4, most preferably 6.0±0.3 or 6.0±0.2 and in particular 6.0±0.1. In another preferred embodiment, the pH is 6.5±1.0 or 6.5±0.8, more preferably 6.5±0.7 or 6.5±0.6, even more preferably 6.5±0.5 or 6.5±0.4, most preferably 6.5±0.3 or 6.5±0.2 and in particular 6.5±0.1. In another preferred embodiment, the pH is 7.0±1.0 or 7.0±0.8, more preferably 7.0±0.7 or 7.0±0.6, even more preferably 7.0±0.5 or 7.0±0.4, most preferably 7.0±0.3 or 7.0±0.2 and in particular 7.0±0.1. In another preferred embodiment, the pH is 7.4±1.0 or 7.4±0.8, more preferably 7.4±0.7 or 7.4±0.6, even more preferably 7.4±0.5 or 7.4±0.4, most preferably 7.4±0.3 or 7.4±0.2 and in particular 7.4±0.1.

The inventive dialysis solution contains one or more inventive copolymers (e.g., two, three, four or five), where the inventive copolymers are defined above.

The inventive dialysis solution contains the inventive copolymer in a total concentration of preferably 0.010 mM to 1.0 M or 0.01 to 750 mM, more preferably 0.10 to 500 mM, even more preferably 1.0 to 250 mM, most preferably 10 to 100 mM and in particular 15 to 90 mM. In a preferred embodiment, the total concentration of inventive copolymer is 25±24 mM, more preferably 25±20 mM, even more preferably 25±15 mM, most preferably 25±10 mM and in particular 25±5 mM. In another preferred embodiment, the total concentration of inventive copolymer is 50±25 mM, more preferably 50±20 mM, even more preferably 50±15 mM, most preferably 50±mM and in particular 50±5 mM. In another preferred embodiment, the total concentration of inventive copolymer is 75±25 mM, more preferably 75±20 mM, even more preferably 75±15 mM, most preferably 75±10 mM and in particular 75±5 mM. In another preferred embodiment, the total concentration of inventive copolymer is 100±25 mM, more preferably 100±20 mM, even more preferably 100±mM, most preferably 100±10 mM and in particular 100±5 mM. The total concentration is preferably calculated by means of the average molecular weight of the inventive copolymers.

The inventive dialysis solution contains the inventive copolymer in a total concentration by weight of preferably 0.01 g l⁻¹ to 1.0 kg l⁻¹, more preferably of 0.1 to 750 g l⁻¹, even more preferably of 1.0 to 500 g l⁻¹, most preferably 10 to 250 g l⁻¹ and in particular of 100 to 200 g l⁻¹. In a preferred embodiment, the total concentration by weight is 25±24 g l⁻¹, more preferably 25±20 g l⁻¹, even more preferably 25±15 g l⁻¹, most preferably 25±10 g l⁻¹ and in particular 25±5 g l⁻¹. In another preferred embodiment, the total concentration by weight is 50±25 g l⁻¹, more preferably 50±20 g l⁻¹, even more preferably 50±15 g l⁻¹, most preferably 50±10 g l⁻¹ and in particular 50±5 g l⁻¹. In another preferred embodiment, the total concentration by weight is 75±25 g l⁻¹, more preferably 75±20 g l⁻¹, even more preferably 75±15 g l⁻¹, most preferably 75±10 g l⁻¹ and in particular 75±5 g l⁻¹. In another preferred embodiment, the total concentration by weight is 100±25 g l⁻¹, more preferably 100±20 g l⁻¹, even more preferably 100±15 g l⁻¹, most preferably 100±10 g l⁻¹ and in particular 100±5 g l⁻¹. In another preferred embodiment, the total concentration by weight is 200±25 g l⁻¹, more preferably 200±20 g l⁻¹, even more preferably 200±15 g l⁻¹, most preferably 200±10 g l⁻¹ and in particular 200±5 g l⁻¹.

The inventive dialysis solution may also contain other osmotically active substances such as glucose, polyglucose, mannitol or glycerol.

The inventive dialysis solution preferably contains one or more electrolytes.

In the sense of this invention, the term “electrolyte” stands for a substance which contains free ions and has electric conductivity. The electrolyte preferably dissociates completely into cations and anions without significantly altering the pH of an aqueous composition. This property differentiates electrolytes from buffer substances. The electrolytes are preferably present in a concentration that results in essentially complete dissociation in water.

Preferred electrolytes are selected from the group of alkali metals, for example, Na⁺ and K⁺, and the alkaline earth metals, for example, Ca²⁺ and Mg²⁺. A preferred anion is Cl⁻.

The inventive dialysis solution may contain additional anions, for example, bicarbonate, dihydrogen phosphate, hydrogen phosphate, phosphate, acetate, lactate and pyruvate; however, these anions (in suitable combination with cations) are not referred to as electrolytes in the sense of this invention but instead are referred to as buffers because of their buffering capacity.

In a preferred embodiment, the inventive dialysis solution contains Na⁺ ions. The concentration of Na⁺ ions is preferably 10 to 200 mM or 50 to 190 mM, more preferably 100 to 180 mM or 110 to 170 mM, even more preferably 115 to 165 mM or 120 to 160 mM, most preferably 125 to 155 mM and in particular 130 to 150 mM. In another preferred embodiment, the inventive dialysis solution does not contain any Na⁺ ions.

In a preferred embodiment, the inventive dialysis solution contains K⁺ ions. The concentration of K⁺ ions is preferably 0.10 to 20 mM, more preferably 0.25 to 15 mM, even more preferably 0.50 to 10 mM, most preferably 0.75 to 7.5 mM and in particular 1.0 to 5.0 mM. In another preferred embodiment, the concentration of K⁺ ions is 1.0±0.75, 2.0±0.75, 3.0±0.75, 4.0±0.75 or 5.0±0.75 mM and in particular 1.0±0.50, 2.0±0.50, 3.0±0.50, 4.0±0.50 or 5.0±0.50. In another preferred embodiment, the inventive dialysis solution does not contain any K⁺ ions.

In a preferred embodiment, the inventive dialysis solution contains Ca²⁺ ions. The concentration of Ca²⁺ ions is preferably 0.10 to 10 mM, more preferably 0.20 to 7.5 mM, even more preferably 0.30 to 5.0 mM, most preferably 0.40 to 2.5 mM and in particular 0.50 to 2.0 mM. In another preferred embodiment, the concentration of Ca²⁺ ions is 1.0±0.75, 2.0±0.75, 3.0±0.75, 4.0±0.75 or 5.0±0.75 mM and in particular 1.0±0.50, 2.0±0.50, 3.0±0.50, 4.0±0.50 or 5.0±0.50. In another preferred embodiment, the inventive dialysis solution does not contain any Ca²⁺ ions.

In a preferred embodiment, the inventive dialysis solution contains Mg²⁺ ions. The concentration of Mg²⁺ ions is preferably 0.010 to 10 mM, more preferably 0.050 to 7.5 mM, even more preferably 0.10 to 5.0 mM, most preferably 0.20 to 2.5 mM and in particular 0.25 to 1.5 mM. In another preferred embodiment, the concentration of Ca²⁺ ions is 0.25±0.20, 0.30±0.20, 0.40±0.20, 0.50±0.20 or 1.0±0.20 mM and in particular 0.25±0.10, 0.30±0.10, 0.40±0.10, 0.50±0.10 or 1.0±0.10 mM. In another preferred embodiment, the inventive dialysis solution does not contain any Mg²⁺ ions.

In a preferred embodiment, the inventive dialysis solution contains Cl⁻ ions. The concentration of Cl⁻ ions is preferably 10 to 300 mM, more preferably 25 to 250 mM, even more preferably 50 to 200 mM, most preferably 75 to 150 mM and in particular 80 to 125 mM. In another preferred embodiment, the inventive dialysis solution does not contain any Cl⁻ ions.

The inventive dialysis solution preferably contains one or more buffers.

Suitable buffers are familiar to the person skilled in the art. Buffers usually comprise lactate, bicarbonate, carbonate, dihydrogen phosphate, hydrogen phosphate, phosphate, pyruvate, citrate, isocitrate, succinate, fumarate, acetate and lactate salts. The person skilled in the art will be aware that the corresponding cation of the anions listed above is a component of the buffer, which is used to adjust the pH (e.g., Na⁺ as a component of the buffer NaHCO₃). However, if the buffer salt has dissociated in water, it also has the effect of an electrolyte. For the purposes of this description, the concentrations of cations or anions and the total concentration an ions are calculated depending on whether they are used as a component of electrolytes, buffers or other compounds (e.g., as a salt of the inventive copolymers).

In a preferred embodiment, the buffer contains bicarbonate. Bicarbonate is a buffer system that is tolerated well and is in equilibrium with H₂CO₃ and CO₂ in an acidic medium and is in equilibrium with carbonate in an alkaline medium. In addition to bicarbonate, other buffer systems which have a buffering effect in the pH range around pH 7 may also be used, including, for example, compounds which are metabolized to bicarbonate in the body such as lactate or pyruvate.

In another preferred embodiment, the buffer contains the salt of a weak acid, preferably lactate. The acid strength (pKₛ) of the weak acid is preferably ≦5. The buffer may also be a mixture of substances having a buffering effect, for example, a mixture containing bicarbonate and a salt of a weak acid (e.g., lactate). A low bicarbonate concentration has the advantage that the CO₂ pressure in the container is low. A traditional polyolefin film may be used as the CO₂ barrier.

In a preferred embodiment, the inventive dialysis solution is buffered by bicarbonate. The bicarbonate concentration is preferably 1.0 to 200 mM, more preferably 5.0 to 150 mM, even more preferably 10 to 100 mM, most preferably 20 to 75 mM or 25 to 50 mM and in particular 30 to 40 mM. In another preferred embodiment, the inventive dialysis solution does not contain any bicarbonate.

In a preferred embodiment, the inventive dialysis solution is buffered by lactate. The lactate concentration is preferably 1.0 to 200 mM, more preferably 5.0 to 150 mM, even more preferably 10 to 100 mM, most preferably 20 to 75 mM or 25 to 50 mM and in particular 30 to 40 mM. In another preferred embodiment, the inventive dialysis solution does not contain any lactate.

In a preferred embodiment, the inventive dialysis solution is buffered by acetate. The acetate concentration is preferably 1.0 to 100 mM, more preferably 1.0 to 50 mM, even more preferably 1.0 to 25 mM, most preferably 1.0 to 10 mM or 2.0 to 7.5 mM and in particular 2.5 to 7.0 mM. In another preferred embodiment, the inventive dialysis solution does not contain any acetate.

The total volume of the dialysis solution is not limited. The volume usually amounts to several liters (suitable volume for administration to a patient) up to several hundred liters (suitable storage volume for more than one patient).

As already explained above, the term “dialysis solution” in the sense of this invention is understood to refer to a ready-to-use dialysis solution, i.e., the dialysis solution may be used directly for the dialysis treatment (hemodialysis or peritoneal dialysis).

In a preferred embodiment, the inventive dialysis solution is a peritoneal dialysis solution as described below.

The peritoneal dialysis solution is biochemically adjusted so that it essentially corrects the metabolic acidosis associated with renal insufficiency. The peritoneal dialysis solution contains bicarbonate preferably in approximately physiological concentrations. In a preferred embodiment, the peritoneal dialysis solution contains bicarbonate in a concentration of approximately 20 to 30 mM. In another preferred embodiment, the peritoneal dialysis solution has a bicarbonate concentration of 25 mM.

Furthermore, the peritoneal dialysis solution preferably contains carbon dioxide with a partial pressure (pCO₂) of less than 60 mmHg. In a preferred embodiment, the pCO₂ of the peritoneal dialysis solution is essentially equal to the pCO₂ measured in blood vessels.

Furthermore, the peritoneal dialysis solution preferably has a pH of approximately 7.4. Therefore, the peritoneal dialysis solution is a physiologically tolerable solution.

The peritoneal dialysis solution preferably is contains a weak acid with a pKₛ 5. The weak acids are preferably compounds which occur as physiological metabolites in the glucose metabolism. The weak acid is preferably selected from the group consisting of lactate, pyruvate, citrate, isocitrate, ketoglutarate, succinate, fumarate, malate and oxaloacetate. These acids may be present either alone or as a mixture in the peritoneal dialysis solution. The weak acids are preferably present in the peritoneal dialysis solution in a concentration of 10 to 20 meq l⁻¹ and especially as sodium salts. In the peritoneal dialysis solution the weak acid preferably present in an amount corresponding to the daily metabolic hydrogen production of approximately 1 meq/kg daily.

The peritoneal dialysis solution contains at least one inventive copolymer as defined above.

The inventive peritoneal dialysis solution preferably contains a concentration of bicarbonate and has a pCO₂ similar to that measured in healthy patients not in renal insufficiency. The weak acid diffuses from the dialysis solution into the bloodstream of the dialysis patient along the concentration gradient and thus corrects the metabolic acidosis of the dialysis patient.

Another subject matter of this invention relates to multicomponent systems for preparing the ready-to-use dialysis solutions described above. They are preferably prepared by a procedure which is described in detail, i.e., by following appropriate instructions (protocol). Said production may take place manually, e.g., by mixing individual components or diluting a component with water. However, the preparation may also take place automatically, e.g., by means of a device which is suitable for this process and may be available commercially. The preparation need not necessarily result in a dialysis solution having a static (uniform) composition but may also result in a dialysis solution, which changes its composition continuously, such that this change can be monitored by a suitable device. For example, the inventive copolymer may be present in a dialysis solution, which is diluted continuously during the dialysis treatment, so the patient is exposed to a declining copolymer concentration.

In a preferred embodiment, the inventive dialysis solutions are suitable for use in the treatment of renal insufficiency.

In another preferred embodiment, the inventive dialysis solutions are suitable for use in dialysis treatment.

In another preferred embodiment, the inventive dialysis solutions are suitable for use in hemodialysis and/or peritoneal dialysis treatment.

Another subject matter of this invention relates to a kit which is configured for preparing the inventive dialysis solutions described above, such that the kit comprises:
a first component,
a second component and
optionally one or more additional components, and
   the inventive dialysis solution can be prepared by mixing the first component with the second component and optionally the additional component(s).

This kit comprises at least one first component and one second component. The kit may also comprise additional components, for example, a third and a fourth component. The kit preferably consists of two components, which are preferably different from one another.

In the sense of this invention, the term “component” comprises liquid, semisolid or solid compositions, which may be the same as or different from one another, such that the inventive ready-to-use dialysis solution is obtained by mixing all of the components.

The first and the second components, independently of one another, may be solid, semisolid or liquid. In the event the components are liquid, they may be solutions or dispersions (e.g., dispersions or suspensions).

In a preferred embodiment, the first component is liquid, preferably pure water or an aqueous solution, and the second component is also liquid. In another preferred embodiment, the first component is liquid, preferably pure water or an aqueous solution, and the second component is solid, preferably a powdered mixture.

The first component is preferably is solution containing osmotically active substances (e.g., the inventive copolymer), calcium ions, magnesium ions, hydronium ions and chloride ions.

The inventive kit may be designed in various ways. For example, the individual components may be present in separate containers (e.g., individual bags). However, the inventive kit is preferably is a multichamber container system (e.g., a flexible or rigid multichamber container system), preferably a flexible multichamber bag system.

The inventive kit is preferably multichamber container system containing the first component, the second component and optionally one or more additional components in chambers, which are separated from one another by soluble and/or breakable partition systems (e.g., breakable partition parts), such that the first component, the second component and optionally the one or more additional components can be mixed together after dissolving and/or breaking the separation system in order to obtain the inventive dialysis solution.

The multichamber container may be in the form of a plastic container (e.g., multichamber plastic bag), each containing a separate chamber for each individual component. The plastic container preferably contains the individual component solutions in chambers, each being separated from the others by partition elements.

The multichamber container is preferably a dual-chamber bag having a first chamber and a second chamber, where the chambers are separated from one another by a soluble and/or breakable partition system, and the first chamber contains the first component and the second chamber contains the second component. The dissolving and/or breaking of the partition system results in mixing of the two components and yields the ready-to-use dialysis solution. The first chamber and the second chamber are preferably arranged adjacent to one another in the multichamber container and are separated from one another by the partition system. The partition system is preferably a dividing seam (e.g., soluble or breakable weld). The dividing seam preferably opens when a pressure is applied to one of the chambers, whereupon the dividing seam breaks or dissolves and the contents of the two chambers thus become mixed and the mixture can be used as a ready-to-use dialysis solution in the dialysis treatment.

The first component of the inventive kit is preferably a sterile solution containing an acid and having a pH≦6.0. The second component is also preferably a sterile solution, preferably containing a buffer and having a pH≧7.0.

The inventive copolymer may be contained in the first component or in the second component as well as in both components in the same or different concentrations. In a preferred embodiment, the inventive copolymer is contained only in the first (acidic) component. In another preferred embodiment, the inventive copolymer is contained only in the second (basic) component. The first component and/or the second component and/or the optional additional component(s) may contain one or more electrolytes.

The person skilled in the art will be aware that mixing the individual components usually involves a dilution effect for the case when the components contain the ingredients in different concentrations. For example, if the inventive copolymer is contained exclusively in one component, then mixing this component with at least one other component will result in an increase in volume with respect to the copolymer component and will thus result in dilution, i.e., a reduction in the copolymer concentration. Consequently the component then contains the inventive copolymer in a higher concentration than that found in the ready-to-use dialysis solution.

The concentration of the inventive copolymer in the component is preferably close to the saturation concentration at a temperature of 5° C. in order to ensure an adequate stability in storage at higher temperatures.

In a preferred embodiment, the total concentration by weight of the inventive copolymer in the component is 0.01 g l⁻¹ to 1.0 kg l⁻¹, more preferably 0.1 to 750 g even more preferably 1.0 to 500 g l⁻¹, most preferably 10 to 250 g l⁻¹ and in particular 100 to 200 g l⁻¹. In another preferred embodiment, the total concentration by weight of the inventive copolymer in the component is 25±24 g l⁻¹, more preferably 25±20 g l⁻¹, even more preferably 25±15 g l⁻¹, most preferably 25±10 g l⁻¹ and in particular 25±5 g l⁻¹. In another preferred embodiment, the total concentration by weight of the inventive copolymer in the component is 50±25 g l⁻¹, more preferably 50±20 g l⁻¹, even more preferably 50±15 g l⁻¹, most preferably 50±10 g l⁻¹ and in particular 50±5 g l⁻¹. In another preferred embodiment, the total concentration by weight of the inventive copolymer in the component is 75±25 g l⁻¹, more preferably 75±20 g even more preferably 75±15 g l⁻¹, most preferably 75±10 g l⁻¹ and in particular 75±5 g l⁻¹. In another preferred embodiment, the total concentration by weight of the inventive copolymer in the component is 100±25 g l⁻¹, more preferably 100±20 g l⁻¹, even more preferably 100±15 g l⁻¹, most preferably 100±10 g l⁻¹ and in particular 100±5 g l⁻¹. In another preferred embodiment, the total concentration by weight of the inventive copolymer in the component is 200±25 g l⁻¹, more preferably 200±20 g l⁻¹, even more preferably 200±15 g l⁻¹, most preferably 200±10 g l⁻¹ and in particular 200±5 g l⁻¹.

In a preferred embodiment, the second component contains the total amount of inventive copolymer, which adjusts the pH of the second component to more than 7.0, more preferably to more than 7.5, even more preferably to more than 8.0, most preferably to more than 8.5 and in particular to more than 9.0. This may preferably be accomplished by using bicarbonate ions, which may be present, for example, in the form of dissociated sodium bicarbonate and/or potassium bicarbonate. In another preferred embodiment, the second component is solid and comprises a powdered mixture containing at least one inventive copolymer and at least one buffer, for example, sodium and/or potassium bicarbonate.

The multichamber bag is preferably suitable for preparing a dialysis solution, which can be used in peritoneal dialysis treatment and which contains the following ingredients, preferably in following concentrations:
Ca²⁺ 0.5 to 5 meq l⁻¹;
Mg²⁺ 0 to 3.0 meq l⁻¹;
Cl⁻ 90.5 to 121 meq l⁻¹;
K⁺ 0 to 4.0 meq l⁻¹;
HCO₃⁻ 25 to 40 meq L⁻¹;
   where one chamber of the multichamber bag system contains a first acidic concentrate and another chamber contains a second basic concentrate, the acidic concentrate containing Ca²⁺ ions and the basic concentrate containing HCO₃⁻ ions but no Ca²⁺ ions, and the two concentrates can be mixed together after the partition system has been dissolved or broken (e.g., dividing seam), such that the mixing of the two concentrates leads to the preparation of the ready-to-use dialysis solution, and the pH of the ready-to-use dialysis solution is 7.2 to 7.4.

The basic concentrate preferably contains at least one inventive copolymer, whereas the acidic concentrate does not contain any inventive copolymer and does not contain any additional osmotically active polymer (e.g., polyglucose).

The basic concentrate preferably contains an amount of bicarbonate that leads to a bicarbonate concentration of the ready-to-use dialysis solution of at least 20 mM. The bicarbonate concentration of the basic component is preferably so high that the ready-to-use dialysis solution has a bicarbonate concentration of 25 to 40 mM.

The pH of the basic buffered second concentrate is preferably adjusted with hydrochloric acid.

The two concentrates are preferably mixed together in a volume ratio of 10:1 to 1:10 or 8:1 to 1:8, more preferably 5:1 to 1:5 or 3:1 to 1:3, even more preferably 2:1 to 1:2 and in particular 1:1.

Another subject matter relates to a process for preparing a dialysis solution in which the desired mixing ratio is automatically achieved by a dialysis machine or a peritoneal dialysis cycler.

In a preferred embodiment, the invention relates to a solid composition, which is suitable for preparing the inventive dialysis solution by dissolving in a defined volume of a solvent (e.g., water). The solid composition is preferably a component as described above and is thus a component of the inventive kit.

The inventive solid composition preferably contains a bicarbonate salt such as sodium or potassium bicarbonate. The substance quantity ratio of bicarbonate to the inventive copolymer in the solid composition is preferably 1:100 to 100:1, more preferably 1:50 to 50:1, even more preferably 1:25 to 25:1, most preferably 1:10 to 10:1 and in particular 1:5 to 5:1.

The inventive volume of solvent needed to prepare the inventive dialysis solution by dissolving the solid composition is preferably 1.0 to 2000 liters. The solvent is preferably purified water, sterilized water or water for injection purposes which optionally contains one or more of the electrolytes described above, one or more osmotically active substances (e.g., at least one inventive copolymer) and/or one or more of the buffers described above.

Another subject matter of this invention relates to the use of at least one inventive copolymer to prepare the inventive dialysis solution (hemodialysis solution or peritoneal dialysis solution), where the dialysis solution is preferably suitable for use in the treatment of diseases of the urogenital system, more preferably for use in dialysis treatment, and in particular for use in hemodialysis or peritoneal dialysis treatment.

Another subject matter of this invention relates to the use of an inventive kit for preparing the inventive dialysis solution (hemodialysis solution or peritoneal dialysis solution), where the dialysis solution is preferably suitable for use in the treatment of diseases of the urogenital system, more preferably for use in dialysis treatment and in particular for use in hemodialysis or peritoneal dialysis treatment.

Another subject matter of this invention relates to the use of an inventive solid composition for preparing the inventive dialysis solution (hemodialysis solution or peritoneal dialysis solution), where the dialysis solution is preferably suitable for use in the treatment of diseases of the urogenital system, more preferably for use in dialysis treatment and in particular for use in hemodialysis treatment or peritoneal dialysis treatment.

### EXAMPLES

### Example 1

β-Cyclodextrin (2 g) is dissolved in anhydrous dimethylformamide (10 mL). Epichlorohydrin (1 mmol) is added slowly to this solution. The resulting solution is stirred for four hours at 60° C. and then cooled over water. Ethanol is added to this cooled solution, and the resulting precipitate is filtered out, washed with cold ethanol and then dried in vacuo.

### Example 2

The sodium salt of (2-carboxyethyl)-β-cyclodextrin with a degree of substitution of approximately 2.5 (3 g) is dissolved in anhydrous dimethylformamide (10 mL). Epichlorohydrin (1.2 mmol) is added slowly to this solution. The resulting solution is stirred overnight at 60° C. and then cooled to room temperature. The solution is concentrated in a rotary evaporator. The resulting precipitate is filtered out, washed with cold water and then dried in vacuo.

### Example 3

6-O-Maltosyl-β-cyclodextrin with a degree of substitution of 1.5 (3 g) is dissolved in 10 mL anhydrous dimethylformamide. Tetramethylene diisocyanate (1.6 mmol) is added slowly to this solution. The resulting solution is stirred overnight at 60° C. and then cooled to room temperature. The solution is concentrated in a rotary evaporator. The resulting precipitate is filtered out, washed with cold ethanol and then dried in vacuo.

### Osmotic Activity

A semisaturated solution in distilled water is prepared using exemplary compounds 2. This solution (50 mL) is placed in a chamber of a Pfeffer cell (chamber A), while distilled water (50 mL) is introduced into the other chamber (chamber B) of the Pfeffer cell. The two chambers are connected via a semipermeable membrane (cellulose membrane, cut-off 1500 Da). Within four hours approximately 4.7 mL of the solution goes from chamber B into the solution of chamber A.

1. A copolymer comprising
at least two cyclodextrin monomers and
at least one linker;
for use in the treatment of diseases of the urogenital system.

2. The copolymer according to claim 1, characterized in that the substance quantity proportion of the linkers is 20 to 95 mol %, based on the total substance quantity of the monomers.

3. The copolymer according to claim 1, characterized in that the substance quantity proportion of the cyclodextrin monomers is 5.0 to 80 mol %, based on the total substance quantity of the monomers.

4. The copolymer according to claim 1, characterized in that the sum of the substance quantity proportion of the linkers and the substance quantity proportion of the cyclodextrin monomers is ≧25 mol %.

5. The copolymer according to claim 1, characterized in that the water solubility of the copolymers at 23° C. is ≧5.0 g l⁻¹.

6. The copolymer according to claim 1, characterized in that the osmolality of a 50 mM aqueous solution of copolymer is ≧50 mosm kg⁻¹.

7. The copolymer according to claim 1, characterized in that the cyclodextrin monomers, which may be the same or different, are derived from α-, β- or γ-cyclodextrin compounds, each of which may be unsubstituted or mono- or polysubstituted with the same or different radicals.

8. The copolymer according to claim 7, characterized in that the radicals are derived from at least one functionalizing agent.

9. The copolymer according to claim 1, characterized in that the linkers are derived from bifunctional and/or polyfunctional crosslinking agents.

10. The copolymer according to claim 1, for use in treatment of renal insufficiency.

11. The copolymer according to claim 1, for use in dialysis treatment.

12. A dialysis solution containing at least one polymer according to claim 1.

13. A kit configured for preparing the dialysis solution according to claim 12, comprising
a first component,
a second component and
optionally one or more additional components,
wherein the dialysis solution according to claim 12 is obtained by mixing the first component with the second component and optionally with the additional component(s).

14. A solid composition suitable for preparing the dialysis solution according to claim 12, characterized in that the dialysis solution according to claim 12 is obtained by dissolving the solid composition in a solvent.

15. A use of at least one copolymer having at least two cyclodextrin monomers and at least one linker to prepare the dialysis solution according to claim 12.

16. A use of a kit comprising
a first component
a second component and
optionally one or more additional components,
wherein the first component is mixed with the second component and optionally with additional component(s) to prepare the dialysis solution according to claim 12.

17. A use of a solid composition according to claim 14 to prepare a dialysis solution having at least two cyclodextrin monomers and at least one linker.
